SEQUENCE LISTING (169 sequences)

SEQ ID NO: 1 - protein, 641 aa, synthetic construct
MTGFVERPEQAHTPNCTGVQYAAMYERSLADPDGFWLEQAKRLDWTQQPRKGGEWSYDPVDIKWFADGSLNLCHNAVDRHLDSRGDTPAIIFEPDDPATPSRTLTYRQLHSEVIHMANALKAIGVTKGERVTIYMPNIVEGVTAMLACARLGAIHSVVFGGFSPEALAGRIIDCESRFVVTADEGKRGAKSVPLKANVDAALEVEGVDVTGVLVVQHTGLAVPMTEGRDHWFHEVKSDADVPCETMAAEDPLFILYTSGSTGKPKGVLHTTGGYGVWTATTFSYIFDYQPGEVFWCTADIGWVTGHSYIVYGPLQNGATQVLFEGVPNYPDFGRFWDVVAKHKVSILYTAPTAIRALMREGDDYVTSRDRSSLRLLGSVGEPINPEAWRWYFDVVGEGRCPIIDTWWQTETGGCMITTLPGAHDMKPGSAGLPMFGIRPQLVDNDGAVLDGATEGNLCITHSWPGQARSVYGDHDRFVQTYFSTYSGKYFTGDGCKRDEDGYYWITGRVDDVINVSGHRMGTAEVESALVLHPQVAEAAVVGYPHDVKGQGIYCYVTTNAGVEGSDELYQELRAHVRKEIGPIATPDQIQFTDGLPKTRSGKIMRRILRKVAENDYGSLGDTSTLADPSLVDRLIEGRQKT

SEQ ID NO: 2 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIAVVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGTQEAAFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEAYNVYSRRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 3 - DNA, 28 bp, synthetic construct
  source 1..28: mol_type = other DNA
cgattggttgccacnnngttggtctgcg

SEQ ID NO: 4 - DNA, 22 bp, synthetic construct
  source 1..22: mol_type = other DNA
gtggcaaccaatcgcctgaaca

SEQ ID NO: 5 - DNA, 27 bp, synthetic construct
  source 1..27: mol_type = other DNA
ccactatgttggtctgnnncctaccgc

SEQ ID NO: 6 - DNA, 26 bp, synthetic construct
  source 1..26: mol_type = other DNA
cagaccaacatagtggcaaccaatcg

SEQ ID NO: 7 - DNA, 23 bp, synthetic construct
  source 1..23: mol_type = other DNA
tgttggtctgcgtnnnaccgctg

SEQ ID NO: 8 - DNA, 23 bp, synthetic construct
  source 1..23: mol_type = other DNA
acgcagaccaacatagtggcaac

SEQ ID NO: 9 - DNA, 22 bp, synthetic construct
  source 1..22: mol_type = other DNA
ggtctgcgtcctnnngctgctg

SEQ ID NO: 10 - DNA, 23 bp, synthetic construct
  source 1..23: mol_type = other DNA
aggacgcagaccaacatagtggc

SEQ ID NO: 11 - DNA, 33 bp, synthetic construct
  source 1..33: mol_type = other DNA
cagaaggtatcgcannngtagcctttactagcg

SEQ ID NO: 12 - DNA, 24 bp, synthetic construct
  source 1..24: mol_type = other DNA
tgcgataccttctgggtgttcacg

SEQ ID NO: 13 - DNA, 32 bp, synthetic construct
  source 1..32: mol_type = other DNA
gaaggtatcgcagttnnngcctttactagcgg

SEQ ID NO: 14 - DNA, 25 bp, synthetic construct
  source 1..25: mol_type = other DNA
aactgcgataccttctgggtgttca

SEQ ID NO: 15 - DNA, 30 bp, synthetic construct
  source 1..30: mol_type = other DNA
ggtatcgcagttgtannntttactagcggc

SEQ ID NO: 16 - DNA, 27 bp, synthetic construct
  source 1..27: mol_type = other DNA
tacaactgcgataccttctgggtgttc

SEQ ID NO: 17 - DNA, 28 bp, synthetic construct
  source 1..28: mol_type = other DNA
gcagttgtagcctttnnnagcggcacca

SEQ ID NO: 18 - DNA, 27 bp, synthetic construct
  source 1..27: mol_type = other DNA
aaaggctacaactgcgataccttctgg

SEQ ID NO: 19 - DNA, 24 bp, synthetic construct
  source 1..24: mol_type = other DNA
cacccctaaagnnncggcccactc

SEQ ID NO: 20 - DNA, 21 bp, synthetic construct
  source 1..21: mol_type = other DNA
gcctttaggggtgccagtggt

SEQ ID NO: 21 - DNA, 26 bp, synthetic construct
  source 1..26: mol_type = other DNA
taaaggcgttgccnnntcctctaccg

SEQ ID NO: 22 - DNA, 20 bp, synthetic construct
  source 1..20: mol_type = other DNA
ggcaacgcctttaggggtgc

SEQ ID NO: 23 - DNA, 25 bp, synthetic construct
  source 1..25: mol_type = other DNA
cccactcctctaccnnnatgagcgc

SEQ ID NO: 24 - DNA, 20 bp, synthetic construct
  source 1..20: mol_type = other DNA
ggtagaggagtgggcaacgc

SEQ ID NO: 25 - DNA, 28 bp, synthetic construct
  source 1..28: mol_type = other DNA
ctcctctaccgctnnnagcgcttgtgtg

SEQ ID NO: 26 - DNA, 21 bp, synthetic construct
  source 1..21: mol_type = other DNA
agcggtagaggagtgggcaac

SEQ ID NO: 27 - DNA, 26 bp, synthetic construct
  source 1..26: mol_type = other DNA
gctatgagcgctnnngtggatgctgc

SEQ ID NO: 28 - DNA, 22 bp, synthetic construct
  source 1..22: mol_type = other DNA
agcgctcatagcggtagaggag

SEQ ID NO: 29 - DNA, 27 bp, synthetic construct
  source 1..27: mol_type = other DNA
cttgtgtggatgctnnngtttccatgt

SEQ ID NO: 30 - DNA, 23 bp, synthetic construct
  source 1..23: mol_type = other DNA
agcatccacacaagcgctcatag

SEQ ID NO: 31 - DNA, 23 bp, synthetic construct
  source 1..23: mol_type = other DNA
tgcggtttccatgnnnggtcgcg

SEQ ID NO: 32 - DNA, 22 bp, synthetic construct
  source 1..22: mol_type = other DNA
catggaaaccgcagcatccaca

SEQ ID NO: 33 - DNA, 29 bp, synthetic construct
  source 1..29: mol_type = other DNA
gtttcctgatcccgnnncctctgtctgac

SEQ ID NO: 34 - DNA, 21 bp, synthetic construct
  source 1..21: mol_type = other DNA
cgggatcaggaaacgccaagg

SEQ ID NO: 35 - DNA, 27 bp, synthetic construct
  source 1..27: mol_type = other DNA
tcctgatcccgatcnnnctgtctgacc

SEQ ID NO: 36 - DNA, 23 bp, synthetic construct
  source 1..23: mol_type = other DNA
gatcgggatcaggaaacgccaag

SEQ ID NO: 37 - DNA, 25 bp, synthetic construct
  source 1..25: mol_type = other DNA
cgatccctctgtctnnnctgggtgg

SEQ ID NO: 38 - DNA, 23 bp, synthetic construct
  source 1..23: mol_type = other DNA
agacagagggatcgggatcagga

SEQ ID NO: 39 - DNA, 23 bp, synthetic construct
  source 1..23: mol_type = other DNA
tccctctgtctgacnnnggtggc

SEQ ID NO: 40 - DNA, 24 bp, synthetic construct
  source 1..24: mol_type = other DNA
gtcagacagagggatcgggatcag

SEQ ID NO: 41 - DNA, 22 bp, synthetic construct
  source 1..22: mol_type = other DNA
gacctgggtgnnncgctggcac

SEQ ID NO: 42 - DNA, 21 bp, synthetic construct
  source 1..21: mol_type = other DNA
gccacccaggtcagacagagg

SEQ ID NO: 43 - DNA, 23 bp, synthetic construct
  source 1..23: mol_type = other DNA
ctgggtggcgaannngcacagtg

SEQ ID NO: 44 - DNA, 21 bp, synthetic construct
  source 1..21: mol_type = other DNA
ttcgccacccaggtcagacag

SEQ ID NO: 45 - DNA, 24 bp, synthetic construct
  source 1..24: mol_type = other DNA
cgaactggcacagnnnaccctggc

SEQ ID NO: 46 - DNA, 19 bp, synthetic construct
  source 1..19: mol_type = other DNA
ctgtgccagttcgccaccc

SEQ ID NO: 47 - DNA, 27 bp, synthetic construct
  source 1..27: mol_type = other DNA
cgttgtgctgctgnnngagttccaacc

SEQ ID NO: 48 - DNA, 20 bp, synthetic construct
  source 1..20: mol_type = other DNA
cagcagcacaacggtaccgc

SEQ ID NO: 49 - DNA, 25 bp, synthetic construct
  source 1..25: mol_type = other DNA
tgtgctgctggaannnttccaaccg

SEQ ID NO: 50 - DNA, 21 bp, synthetic construct
  source 1..21: mol_type = other DNA
ttccagcagcacaacggtacc

SEQ ID NO: 51 - DNA, 25 bp, synthetic construct
  source 1..25: mol_type = other DNA
gctgctggaagagnnncaaccggac

SEQ ID NO: 52 - DNA, 23 bp, synthetic construct
  source 1..23: mol_type = other DNA
ctcttccagcagcacaacggtac

SEQ ID NO: 53 - DNA, 21 bp, synthetic construct
  source 1..21: mol_type = other DNA
gccactcacgtgnnnctggcg

SEQ ID NO: 54 - DNA, 21 bp, synthetic construct
  source 1..21: mol_type = other DNA
cacgtgagtggcacgttcacg

SEQ ID NO: 55 - DNA, 21 bp, synthetic construct
  source 1..21: mol_type = other DNA
ccactcacgtgttcnnngcgc

SEQ ID NO: 56 - DNA, 22 bp, synthetic construct
  source 1..22: mol_type = other DNA
gaacacgtgagtggcacgttca

SEQ ID NO: 57 - DNA, 22 bp, synthetic construct
  source 1..22: mol_type = other DNA
ctcacgtgttcctgnnnccgaa

SEQ ID NO: 58 - DNA, 21 bp, synthetic construct
  source 1..21: mol_type = other DNA
caggaacacgtgagtggcacg

SEQ ID NO: 59 - DNA, 21 bp, synthetic construct
  source 1..21: mol_type = other DNA
tggcgccgaacnnnctgtacc

SEQ ID NO: 60 - DNA, 19 bp, synthetic construct
  source 1..19: mol_type = other DNA
gttcggcgccaggaacacg

SEQ ID NO: 61 - DNA, 22 bp, synthetic construct
  source 1..22: mol_type = other DNA
cgtcgcgttgttnnnggcggtg

SEQ ID NO: 62 - DNA, 22 bp, synthetic construct
  source 1..22: mol_type = other DNA
aacaacgcgacgcagagaagac

SEQ ID NO: 63 - DNA, 22 bp, synthetic construct
  source 1..22: mol_type = other DNA
gtcgcgttgtttacnnnggtgc

SEQ ID NO: 64 - DNA, 24 bp, synthetic construct
  source 1..24: mol_type = other DNA
gtaaacaacgcgacgcagagaaga

SEQ ID NO: 65 - DNA, 22 bp, synthetic construct
  source 1..22: mol_type = other DNA
cgttgtttacgnnncggcaccg

SEQ ID NO: 66 - DNA, 19 bp, synthetic construct
  source 1..19: mol_type = other DNA
gccgtaaacaacgcgacgc

SEQ ID NO: 67 - DNA, 20 bp, synthetic construct
  source 1..20: mol_type = other DNA
tgtttacgnnngtgcaccgg

SEQ ID NO: 68 - DNA, 20 bp, synthetic construct
  source 1..20: mol_type = other DNA
accgccgtaaacaacgcgac

SEQ ID NO: 69 - DNA, 17 bp, synthetic construct
  source 1..17: mol_type = other DNA
cggcggtgcannngcag

SEQ ID NO: 70 - DNA, 20 bp, synthetic construct
  source 1..20: mol_type = other DNA
tgcaccgccgtaaacaacgc

SEQ ID NO: 71 - DNA, 23 bp, synthetic construct
  source 1..23: mol_type = other DNA
cggtgcaccgnnngtaccatctc

SEQ ID NO: 72 - DNA, 19 bp, synthetic construct
  source 1..19: mol_type = other DNA
cggtgcaccgccgtaaaca

SEQ ID NO: 73 - DNA, 25 bp, synthetic construct
  source 1..25: mol_type = other DNA
tgctgtgctgatgnnnaactacggc

SEQ ID NO: 74 - DNA, 24 bp, synthetic construct
  source 1..24: mol_type = other DNA
catcagcacagcacccatacgttc

SEQ ID NO: 75 - DNA, 25 bp, synthetic construct
  source 1..25: mol_type = other DNA
tgtgctgatgcagnnntacggcacc

SEQ ID NO: 76 - DNA, 23 bp, synthetic construct
  source 1..23: mol_type = other DNA
ctgcatcagcacagcacccatac

SEQ ID NO: 77 - DNA, 24 bp, synthetic construct
  source 1..24: mol_type = other DNA
tgctgatgcagaacnnnggcaccc

SEQ ID NO: 78 - DNA, 22 bp, synthetic construct
  source 1..22: mol_type = other DNA
gttctgcatcagcacagcaccc

SEQ ID NO: 79 - DNA, 27 bp, synthetic construct
  source 1..27: mol_type = other DNA
ctgatgcagaactacnnnacccaggaa

SEQ ID NO: 80 - DNA, 24 bp, synthetic construct
  source 1..24: mol_type = other DNA
gtagttctgcatcagcacagcacc

SEQ ID NO: 81 - DNA, 24 bp, synthetic construct
  source 1..24: mol_type = other DNA
gcagaactacgnnncgcaggaagc

SEQ ID NO: 82 - DNA, 23 bp, synthetic construct
  source 1..23: mol_type = other DNA
gccgtagttctgcatcagcacag

SEQ ID NO: 83 - DNA, 24 bp, synthetic construct
  source 1..24: mol_type = other DNA
gaactacggcaccnnngaagcagc

SEQ ID NO: 84 - DNA, 22 bp, synthetic construct
  source 1..22: mol_type = other DNA
ggtgccgtagttctgcatcagc

SEQ ID NO: 85 - DNA, 25 bp, synthetic construct
  source 1..25: mol_type = other DNA
tacggcacccagnnngcagctttca

SEQ ID NO: 86 - DNA, 22 bp, synthetic construct
  source 1..22: mol_type = other DNA
ctgggtgccgtagttctgcatc

SEQ ID NO: 87 - DNA, 26 bp, synthetic construct
  source 1..26: mol_type = other DNA
cacccaggaagcannnttcatcgcag

SEQ ID NO: 88 - DNA, 20 bp, synthetic construct
  source 1..20: mol_type = other DNA
tgcttcctgggtgccgtagt

SEQ ID NO: 89 - DNA, 25 bp, synthetic construct
  source 1..25: mol_type = other DNA
ccaggaagcagctnnnatcgcagca

SEQ ID NO: 90 - DNA, 18 bp, synthetic construct
  source 1..18: mol_type = other DNA
agctgcttcctgggtgcc

SEQ ID NO: 91 - DNA, 26 bp, synthetic construct
  source 1..26: mol_type = other DNA
gtgaactgctgaccnnngtaggtcgt

SEQ ID NO: 92 - DNA, 21 bp, synthetic construct
  source 1..21: mol_type = other DNA
ggtcagcagttcacgacgtgc

SEQ ID NO: 93 - DNA, 33 bp, synthetic construct
  source 1..33: mol_type = other DNA
gtacgttccccgnnnactatgtctggttactgg

SEQ ID NO: 94 - DNA, 22 bp, synthetic construct
  source 1..22: mol_type = other DNA
cggggaacgtacccagacttca

SEQ ID NO: 95 - DNA, 31 bp, synthetic construct
  source 1..31: mol_type = other DNA
acgttccccgatgnnnatgtctggttactgg

SEQ ID NO: 96 - DNA, 22 bp, synthetic construct
  source 1..22: mol_type = other DNA
catcggggaacgtacccagact

SEQ ID NO: 97 - DNA, 28 bp, synthetic construct
  source 1..28: mol_type = other DNA
gctgcgtactggtnnngttggtaccttc

SEQ ID NO: 98 - DNA, 20 bp, synthetic construct
  source 1..20: mol_type = other DNA
accagtacgcagccaaccac

SEQ ID NO: 99 - DNA, 26 bp, synthetic construct
  source 1..26: mol_type = other DNA
tggtcacctgcatnnnaccgatcgtc

SEQ ID NO: 100 - DNA, 25 bp, synthetic construct
  source 1..25: mol_type = other DNA
atgcaggtgaccatcctcatcgaag

SEQ ID NO: 101 - DNA, 25 bp, synthetic construct
  source 1..25: mol_type = other DNA
cctgcatctgaccnnncgtctgcag

SEQ ID NO: 102 - DNA, 23 bp, synthetic construct
  source 1..23: mol_type = other DNA
ggtcagatgcaggtgaccatcct

SEQ ID NO: 103 - DNA, 26 bp, synthetic construct
  source 1..26: mol_type = other DNA
tgcatctgaccgatnnnctgcaggac

SEQ ID NO: 104 - DNA, 24 bp, synthetic construct
  source 1..24: mol_type = other DNA
atcggtcagatgcaggtgaccatc

SEQ ID NO: 105 - DNA, 35 bp, synthetic construct
  source 1..35: mol_type = other DNA
tgcaggacatcatcnnngttgaagcatataacgtc

SEQ ID NO: 106 - DNA, 24 bp, synthetic construct
  source 1..24: mol_type = other DNA
gatgatgtcctgcagacgatcggt

SEQ ID NO: 107 - DNA, 39 bp, synthetic construct
  source 1..39: mol_type = other DNA
ggacatcatcatcgttnnngcatataacgtctattcccg

SEQ ID NO: 108 - DNA, 25 bp, synthetic construct
  source 1..25: mol_type = other DNA
aacgatgatgatgtcctgcagacga

SEQ ID NO: 109 - DNA, 39 bp, synthetic construct
  source 1..39: mol_type = other DNA
catcatcatcgttgaannntataacgtctattcccgtcg

SEQ ID NO: 110 - DNA, 26 bp, synthetic construct
  source 1..26: mol_type = other DNA
ttcaacgatgatgatgtcctgcagac

SEQ ID NO: 111 - DNA, 37 bp, synthetic construct
  source 1..37: mol_type = other DNA
atcatcgttgaagcannnaacgtctattcccgtcgtg

SEQ ID NO: 112 - DNA, 25 bp, synthetic construct
  source 1..25: mol_type = other DNA
tgcttcaacgatgatgatgtcctgc

SEQ ID NO: 113 - DNA, 35 bp, synthetic construct
  source 1..35: mol_type = other DNA
catcgttgaagcatatnnngtctattcccgtcgtg

SEQ ID NO: 114 - DNA, 28 bp, synthetic construct
  source 1..28: mol_type = other DNA
atatgcttcaacgatgatgatgtcctgc

SEQ ID NO: 115 - DNA, 34 bp, synthetic construct
  source 1..34: mol_type = other DNA
gcatataacgtctattccnnncgtgtggaacatg

SEQ ID NO: 116 - DNA, 34 bp, synthetic construct
  source 1..34: mol_type = other DNA
ggaatagacgttatatgcttcaacgatgatgatg

SEQ ID NO: 117 - DNA, 27 bp, synthetic construct
  source 1..27: mol_type = other DNA
atcacctgggtgatnnncacgttcctc

SEQ ID NO: 118 - DNA, 24 bp, synthetic construct
  source 1..24: mol_type = other DNA
atcacccaggtgatcacgaaccag

SEQ ID NO: 119 - DNA, 22 bp, synthetic construct
  source 1..22: mol_type = other DNA
ccatcccggtaactnnngccgg

SEQ ID NO: 120 - DNA, 22 bp, synthetic construct
  source 1..22: mol_type = other DNA
agttaccgggatggagcgaacg

SEQ ID NO: 121 - DNA, 23 bp, synthetic construct
  source 1..23: mol_type = other DNA
cccggtaactcctnnnggcaaac

SEQ ID NO: 122 - DNA, 21 bp, synthetic construct
  source 1..21: mol_type = other DNA
aggagttaccgggatggagcg

SEQ ID NO: 123 - DNA, 30 bp, synthetic construct
  source 1..30: mol_type = other DNA
ggtaactcctgccnnnaaaccagataaagt

SEQ ID NO: 124 - DNA, 22 bp, synthetic construct
  source 1..22: mol_type = other DNA
ggcaggagttaccgggatggag

SEQ ID NO: 125 - DNA, 31 bp, synthetic construct
  source 1..31: mol_type = other DNA
ctcctgccgnnncgccagataaagtgaaagt

SEQ ID NO: 126 - DNA, 18 bp, synthetic construct
  source 1..18: mol_type = other DNA
gccggcaggagttaccgg

SEQ ID NO: 127 - DNA, 30 bp, synthetic construct
  source 1..30: mol_type = other DNA
cctgccggcaaannngataaagtgaaagtg

SEQ ID NO: 128 - DNA, 20 bp, synthetic construct
  source 1..20: mol_type = other DNA
tttgccggcaggagttaccg

SEQ ID NO: 129 - DNA, 29 bp, synthetic construct
  source 1..29: mol_type = other DNA
gccggcaaaccannnaaagtgaaagtgcg

SEQ ID NO: 130 - DNA, 21 bp, synthetic construct
  source 1..21: mol_type = other DNA
tggtttgccggcaggagttac

SEQ ID NO: 131 - DNA, 32 bp, synthetic construct
  source 1..32: mol_type = other DNA
ccactcttacatatggtgtgttgtgctttagc

SEQ ID NO: 132 - DNA, 32 bp, synthetic construct
  source 1..32: mol_type = other DNA
taccactcttacatatggtgtgttgtgcttta

SEQ ID NO: 133 - DNA, 32 bp, synthetic construct
  source 1..32: mol_type = other DNA
tgttaccactcttacatatggtgtgttgtgct

SEQ ID NO: 134 - DNA, 34 bp, synthetic construct
  source 1..34: mol_type = other DNA
ctcttgttaccactcttacatatggtgtgttgtg

SEQ ID NO: 135 - DNA, 36 bp, synthetic construct
  source 1..36: mol_type = other DNA
cctactcttgttaccactcttacatatggtgtgttg

SEQ ID NO: 136 - DNA, 26 bp, synthetic construct
  source 1..26: mol_type = other DNA
tgtaagagtggtaacaagagtaggcc

SEQ ID NO: 137 - DNA, 31 bp, synthetic construct
  source 1..31: mol_type = other DNA
ctcttacatatggtgtgttgtgctttagccg

SEQ ID NO: 138 - DNA, 29 bp, synthetic construct
  source 1..29: mol_type = other DNA
cacaccatatgtaagagtggtaacaagag

SEQ ID NO: 139 - DNA, 30 bp, synthetic construct
  source 1..30: mol_type = other DNA
aatgggatcaaagcatacttcaaaatccgc

SEQ ID NO: 140 - DNA, 29 bp, synthetic construct
  source 1..29: mol_type = other DNA
tgctttgatcccatttttttgcttatcag

SEQ ID NO: 141 - DNA, 30 bp, synthetic construct
  source 1..30: mol_type = other DNA
caatcactaccttagcacacagtcggtatt

SEQ ID NO: 142 - DNA, 25 bp, synthetic construct
  source 1..25: mol_type = other DNA
gctaaggtagtgattgtctggcaac

SEQ ID NO: 143 - DNA, 22 bp, synthetic construct
  source 1..22: mol_type = other DNA
ctgagatacctacagcgtgagc

SEQ ID NO: 144 - DNA, 26 bp, synthetic construct
  source 1..26: mol_type = other DNA
cgtcactcatggtgatttctcacttg

SEQ ID NO: 145 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIASVAFTSGTTGTPKGVAHSSTAMSACVDAAVSMYGRGPWRFLIPSPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGTQEAFFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEAYNVYSLRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 146 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIASVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGCQEAFFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVETYNVYSRRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 147 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIASVAFTSGTTGTPKGVAHSSTAMSACVDAAVSMYGRGPWRFLIPSPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGTQEAFFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEAYNVYSTRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 148 - protein, 517 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIASVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGCQEAFFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVESYNVYSRRVEHVLTEHPDVRAAAVVGVPDPDSGEAVCAAVVVADGADPDPEHLRALVRDHLGDLHVPRRVEFVRIPVTPAGKPDKVKVRTWFTD

SEQ ID NO: 149 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIASVAFTSGTTGTPKGVAHSSTAMSACVDALVSMYGRGPWRFLIPIPLSDLGGELAQFTLATGGTVVLLEEFQPDAVLEAIERERATHVFLAPNWLYQLAEHPALPRSDLSSLRRVVYGGAPAVPSRVAAARERMGAVLMQNYGTQEAAFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEAYNVYSQRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 150 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIAVVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGAPAVPSRVAAARERMGAVLMQQYGTQEALFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEMYNVYSRRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 151 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPYAAVTEQARAIAAADSAALVFEPSVEARAADLLERVSVPVVLSLGPTSRGRDILAASVPEGTPLRYREHPEGIASVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGCPAVPSRVAAARERMGAVLMQNYGTQEAAFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVESYNVYSRRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 152 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIAVVAFTSGTTGTPKGVAHSSTAMSACVDAAVSMYGRGPWRFLIPIPLSDVGGFLAQCTLATGGTVVLLEEFQPDAVLEAIERERATHVFLAPNWLYQLAEHPALPRSDLSSLRRVVYGGAPAVPSRVAAARERMGAVLMQNYGTQEALFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEAYNVYSVRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 153 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIAVVAFTSGTTGTPKGVAHSSTAMSACVDAAVSMYGRGPWRFLIPIPLSDLGGELAQCTLATGGTVVLLEEFQPDAVLEAIERERATHVFLTPNWLYQLAEHPALPRSDLSSLRRVVYGGAPAVPSRVAAARERMGAVLMQNYGTQEAFFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVEAYNVYSVRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 154 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIAVVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGTQEAMFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVESYNVYSRRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 155 - protein, 518 aa, synthetic construct
MEKKIWSHPQFEKGGSGENLYFQGGYARRVMDGIGEVAVTGAGGSVTGARLRHQVRLLAHALTEAGIPPGRGVACLHANTWRAIALRLAVQAIGCHYVGLRPTAAVTEQARAIAAADSAALVFEPSVEARAADLLERVSVPVVLSLGPTSRGRDILAASVPEGTPLRYREHPEGIAAVAFTSGTTGTPKGVAHSSTAMSACVDAAVSMYGRGPWRFLIPIPLSDLGGELAQCTLATGGTVVLLEEFQPDAVLEAIERERATHVFLAPNWLYQLAEHPALPRSDLSSLRRVVYGGAPAVPSRVAAARERMGAVLMQNYGTQEALFIAALTPDDHARRELLTAVGRPLPHVEVEIRDDSGGTLPRGAVGEVWVRSPMTMSGYWRDPERTAQVLSGGWLRTGDVGTFDEDGHLHLTDRLQDIIIVETYNVYSVRVEHVLTEHPDVRAAAVVGVPDPDSGEAVCAAVVVADGADPDPEHLRALVRDHLGDLHVPRRVEFVRSIPVTPAGKPDKVKVRTWFTD

SEQ ID NO: 156 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcagttgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcagctttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaagcatataacgtctattcccgtcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 157 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcaagcgtagcctttactagcggcaccactggcacccctaaaggcgttgcccactcctctaccgctatgagcgcttgtgtggatgctgcggtttccatgtacggtcgcggtccttggcgtttcctgatcccgagccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcatttttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaagcatataacgtctattccctgcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 158 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcaagcgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggctgccaggaagcatttttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaaacctataacgtctattcccgtcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 159 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcaagcgtagcctttactagcggcaccactggcacccctaaaggcgttgcccactcctctaccgctatgagcgcttgtgtggatgctgcggtttccatgtacggtcgcggtccttggcgtttcctgatcccgagccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcatttttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaagcatataacgtctattccacccgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 160 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcaagcgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggctgccaggaagcatttttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaaagctataacgtctattcccgtcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 161 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcaagcgtagcctttactagcggcaccactggcacccctaaaggcgttgcccactcctctaccgctatgagcgcttgtgtggatgctctggtttccatgtacggtcgcggtccttggcgtttcctgatcccgatccctctgtctgacctgggtggcgaactggcacagtttaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcagctttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaagcatataacgtctattcccagcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 162 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcagttgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagcagtacggcacccaggaagcactgttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaaatgtataacgtctattcccgtcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 163 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtccttatgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcaagcgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggttgcccggcagtaccatctcgtgtagcagcagcacgtgaacgtatgggtgctgtgctgatgcagaactacggcacccaggaagcagctttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaaagctataacgtctattcccgtcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 164 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcagttgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacgtgggtggctttctggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcactgttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaagcatataacgtctattccgtgcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 165 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcagttgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctgaccccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcatttttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaagcatataacgtctattccgtgcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 166 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcagttgtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcaatgttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaaagctataacgtctattcccgtcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 167 - DNA, 1557 bp, synthetic construct
  source 1..1557: mol_type = other DNA
  misc_feature 1..72: note = CAT-Strep-Linker fusion tag
atggagaaaaaaatctggagccatccgcagttcgaaaaaggcggatccggagaaaacctgtatttccagggcggttacgctcgtcgtgtaatggatggtatcggtgaagtagcggtaactggcgctggtggttctgtaactggtgcgcgtctgcgccatcaggttcgtctgctggctcatgctctgaccgaagcgggtattccgccaggccgtggtgtagcatgtctgcatgctaacacctggcgtgcgatcgcactgcgtctggctgttcaggcgattggttgccactatgttggtctgcgtcctaccgctgctgttactgaacaggcacgcgcaattgcggctgctgattctgccgcactggttttcgaaccaagcgttgaagctcgtgcagctgacctgctggaacgtgtttctgtgccggttgtgctgtctctgggtccgacctctcgtggccgtgatatcctggcagctagcgttccggaaggtacgccgctgcgttaccgtgaacacccagaaggtatcgcagcggtagcctttactagcggcaccactggcacccctaaaggcgttgcccactcctctaccgctatgagcgcttgtgtggatgctgcggtttccatgtacggtcgcggtccttggcgtttcctgatcccgatccctctgtctgacctgggtggcgaactggcacagtgtaccctggctaccggcggtaccgttgtgctgctggaagagttccaaccggacgccgttctggaagctatcgaacgtgaacgtgccactcacgtgttcctggcgccgaactggctgtaccagctggctgaacatccggctctgccgcgttctgatctgtcttctctgcgtcgcgttgtttacggcggtgcaccggcagtaccatctcgtgtagcagcagcacgtgaacgtatgggtgctgtgctgatgcagaactacggcacccaggaagcactgttcatcgcagcactgactccagacgatcacgcacgtcgtgaactgctgaccgctgtaggtcgtcctctgccacacgttgaggtggaaatccgtgatgactctggtggtactctgccgcgtggtgcggtaggtgaagtctgggtacgttccccgatgactatgtctggttactggcgtgacccggaacgtacggctcaggttctgtctggtggttggctgcgtactggtgatgttggtaccttcgatgaggatggtcacctgcatctgaccgatcgtctgcaggacatcatcatcgttgaaacctataacgtctattccgtgcgtgtggaacatgttctgaccgaacacccagatgttcgcgcagctgcggttgttggcgtaccagatccggactctggtgaagctgtttgcgctgcggttgtagtcgcggatggtgcggatcctgaccctgaacacctgcgtgctctggttcgtgatcacctgggtgatctgcacgttcctcgccgtgttgagttcgttcgctccatcccggtaactcctgccggcaaaccagataaagtgaaagtgcgtacctggttcaccgactaa

SEQ ID NO: 168 - protein, 583 aa, synthetic construct
MTVQAQNIDAITAGAMPHEEPELTDGFHLVIDALKLNGIETIYNVPGIPITDLGRLAQAEGLRVISFRHEQNAGNAAAIAGFLTKKPGICLTVSAPGFLNGLTALANATTNCFPMILISGSSEREIVDLQQGDYGEMDQLAIAKPLCKAAFRVLHAADIGIGVARAIRAAVSGRPGGVYLDLPAKLFSQVIDADLGARSLVKVIDAAPAQLPAPAAIARALDVLKSAERPLIILGKGAAYAQADEAVRALVEESGIPYVPMSMAKGLLPDTHPLSAGAARSTALKDSDVVLLVGARLNWLLSHGKGKTWGEPGSKRFIQIDIEPREMDSNVEIVAPVVGDIGSCVEALLDGIRKDWKGAPSNWLETLRGKREANIAKMAPKLMKNSSPMCFHSALGALRTVIKERPDAILVNEGCNTLDLARGIIDMYQPRKRLDVGTWGVMGIGMGFAVAAAVETGKPVLAVEGDSAFGFSGMEVETICRYELPVCIVIFNNNGIFRGTDTDPTGRDPGTTVFVKNSRYDKMMEAFGGVGVNVTTPDELKRAVDEAMNSGKPTLINAEIDPAAGSEGGNIGSLNPQSTLKKK

SEQ ID NO: 169 - protein, 370 aa, synthetic construct
MLPRLICINDYEQHAKSVLPKSIYDYYRSGANDEETLADNIAAFSRWKLYPRMLRNVAETDLSTSVLGQRVSMPICVGATAMQRMAHVDGELATVRACQSLGTGMMLSSWATSSIEEVAEAGPEALRWLQLYIYKDREVTKKLVRQAEKMGYKAIFVTVDTPYLGNRLDDVRNRFKLPPQLRMKNFETSTLSFSPEENFGDDSGLAAYVAKAIDPSISWEDIKWLRRLTSLPIVAKGILRGDDAREAVKHGLNGILVSNHGARQLDGVPATIDVLPEIVEAVEGKVEVFLDGGVRKGTDVLKALALGAKAVFVGRPIVWGLAFQGEKGVQDVLEILKEEFRLAMALSGCQNVKVIDKTLVRKNPLAVSKI

(19) United States
(12) Patent Application Publication
(10) Pub. No.: US 2025/0259704 A1
(43) Pub. Date: Aug. 14, 2025

(54) SYSTEMS AND METHODS FOR CELL-FREE ITERATIVE SITE SATURATION MUTAGENESIS AND ITS APPLICATION FOR THE DIRECTED EVOLUTION OF ENZYMES CATALYZING UNNATURAL REACTIONS

(71) Applicant: Northwestern University, Evanston, IL (US)

(72) Inventors: Micahel C. Jewett, Evanston, IL (US); Jonathan W. Bogart, Evanston, IL (US); Grant M. Landwehr, Evanston, IL (US); Ashty Stephen Karim, Evanston, IL (US)

(21) Appl. No.: 18/878,530

(22) PCT Filed: Jun. 26, 2023
(86) PCT No.: PCT/US2023/069097

§ 371 Date: Dec. 23, 2024

Related U.S. Application Data
(60) Provisional application No. 63/355,539, filed on Jun. 24, 2022.

(51) Int. Cl. G16B 20/50; C12N 9/00; C12N 15/10; G16B 25/20

(52) U.S. Cl. CPC G16B 20/50; C12N 9/93; C12N 15/1058; C12N 15/1089; G16B 25/20; C12Y 602/01001

(57) ABSTRACT

Disclosed are methods, compositions, systems, and protein compounds for the directed evolution of enzymes and proteins. The method comprising generating variant protein with a desired functionality, comprising one or more DNA expression templates comprising nucleic acid sequences encoding a variant protein, expressing the variant protein using cell-free protein synthesis; and analyzing one or more parameters associated with the variant protein.

### CROSS-REFERENCE TO RELATED APPLICATIONS

This application claims the benefit of U.S. Provisional Application 63/355,539 filed Jun. 24, 2022, the entire content of which is incorporated herein by reference.

### STATEMENT REGARDING FEDERALLY SPONSORED RESEARCH OR DEVELOPMENT

This invention was made with government support under grant DE-SC0018249 awarded by the Department of Energy. The government has certain rights in the invention.

### SEQUENCE LISTING STATEMENT

This application includes a sequence listing in XML format named “702581_02349_SL_ST26.xml” which is 181,182 bytes in size and was created on Jun. 22, 2023. The sequence listing is electronically submitted via Patent Center with the application and is incorporated herein by reference in its entirety.

### BACKGROUND

Enzymes have several properties that make them attractive alternatives to traditional catalysts, such as sustainability, activity under mild conditions, and exhibiting stereo-and chemo-selectivity. Decades of research in attempting to shape the fitness of enzymes to become superior biocatalysts has resulted in a diverse array of directed evolution technologies. However, there still remains a niche to rapidly evolve enzymes with new-to-nature functionalities. This is predominantly due to the low-throughput and labor-intensive methods required to generate and express sequence-defined DNA libraries encoding enzyme mutants of interest. Therefore, there is a need in the art for methods and systems to generate variant proteins with a desired functionality in a high-throughput manner.

### SUMMARY

Disclosed herein are methods, compositions, systems, and protein compounds for the directed evolution of enzymes and proteins. Also disclosed are enzymes evolved by the methods disclosed herein, having altered function, specificity, and/or activity as compared to their wild-type counterparts.

An aspect of the disclosure is a method of generating variant protein with a desired functionality. The method include: (i) generating one or more DNA expression templates comprising nucleic acid sequences encoding a variant protein, wherein the variant protein is based on a reference protein and wherein the protein comprises at least one variant amino acid residue as compared to the reference protein; (ii) expressing the variant protein using cell-free protein synthesis; and (iii) analyzing one or more parameters associated with the variant protein and identifying one or more variant proteins with a desired functionality based on the one or more parameters.

Another aspect of the disclosure is method of generating a variant or mutant protein that includes: (i) generating one or more DNA expression templates comprising a nucleic acid sequence encoding a variant protein, wherein the variant protein is based on a reference protein and wherein the protein comprises at least one variant amino acid residue as compared to the reference protein; (ii) expressing the variant protein using cell-free protein synthesis; (iii) analyzing one or more parameter associated with the variant protein and identifying one or more variant protein with a desired functionality based on the one or more parameter; and (iv) using a computer system having one or more processors, and memory storing one or more programs for execution by the one or more processors: analyzing the one or more variant proteins to develop one or more computer models based on the at least one variant amino acid residues in the one or more variant proteins.

Another aspect is modified amide synthetase (McbA) and modified acly-CoA synthetase enzymes generated by the disclosed methods. The modified enzymes may be used to produce variant protein.

A further aspect is A system comprising a computer system having one or more processors, and memory storing one or more programs for execution by the one or more processors; DNA expression templates comprising nucleic acid sequences encoding variant proteins, wherein the variant proteins are based on a reference protein and wherein the proteins comprise at least one variant amino acid residue as compared to the reference protein; cell-free expression reagents comprising an information template, an energy regeneration system, and salts.

Another aspect is a computer system and a machine learning model used in the disclosed methods, the machine learning model being trained on training data to generate data that predict site selection for directed evolution of a protein.

### BRIEF DESCRIPTION OF THE FIGURES

FIGS. 1A-1B. Schematics of an exemplary cell-free, machine learning-guided protein engineering workflow and how it enables parallelized protein engineering. Phase 1: putative residues directing enzyme catalysis are rationally selected and site saturation mutagenesis and cell-free protein expression are carried out in less than 24 hours to generate sequence-defined libraries. Phase 2: The libraries screened for desirable protein fitness metrics. Phase 3: Iterate through steps to find multiple beneficial mutations. Phase 4: Predict the fitness landscape based on outputs from Phase 2, and then use the fitness landscapes for residue selection in Phase 1 of subsequent cycles. Sample reactive enzyme (FIG. 1A), and wild-type enzyme (FIG. 1B).

FIGS. 2A-2C. Cell-free DNA assembly efficiency is robust to differences in homologous overlaps of mutagenic primers. (FIG. 2A) is a workflow to introduce mutations and generate form linear expression templates (LETs). (FIG. 2B) shows five forward primers containing homologous overlaps with a single reverse primer were designed, each overlap differing in Tₘ of approximately 5° C. The primers for this experiment were used to introduce a stop codon at Y66 in muGFP. (FIG. 2C). DNA gel showing method produces LETs of the correct size. Successful mutagenesis to a stop codon (TAA) was confirmed with Sanger sequencing.

FIGS. 3A-3C. Validation of cell-free protein engineering workflow with muGFP. (FIG. 3A) illustrates crystal structure of monomeric ultra-stable green fluorescent protein (muGFP) with residues targeted for saturation mutagenesis highlighted (PDB: 5JZL). (FIG. 3B) shows green fluorescence of single mutants of muGFP generated by site saturation mutagenesis. Data are showing mean fluorescence for n=3 replicates normalized to wild type (wt-muGFP). (FIG. 3C) is an autoradiogram of selected mutants (denoted with asterisks in FIG. 3B) showing full-length, soluble proteins are expressed, except in the case when the mutation is a premature stop codon.

FIGS. 4A-4F. Optimized reaction conditions for cell-free DNA assembly enable site-saturation mutagenesis. (FIG. 4A) shows the designed primers for the site saturation of muGFP Y66 with NNN indicating the targeted codon. (FIG. 4B) are DNA gels demonstrating the expected product size for the two PCR steps of the site saturation of muGFP Y66. Top: amplified linear full plasmid template with the integrated mutation; bottom: amplified linear expression template (LET). (FIG. 4C) amino acid table containing all codons used for site saturation mutagenesis in this study, numbered according to the gels in FIG. 4B. The codons correspond to the most prevalent codons found in E. coli and were held constant. FIG. 4D is sequence alignment of muGFP four residue site saturation; the Sanger sequence alignment for every muGFP mutant found in FIG. 3B generated using the cell-free DNA assembly method. FIGS. 4E-4F show complete protein gels of the truncated image shown in FIG. 3A depicting selected muGFP mutants from the site saturation mutagenesis screen. Mutants were expressed using CFPS with a radioactive leucine (¹⁴C-leucine) to distinguish the expressed protein among the e. coli proteome present in CFPS. Two residues were selected (Y66 and Y164) and a stop codon (Y66*) was included as a control to produce a truncated protein. FIG. 4E shows Coomassie stained protein gel of muGFP expressed in CFPS. FIG. 4F shows the same gel imaged as an autoradiogram.

FIGS. 5A-5F. Engineering campaign for the current FDA-approved drug moclobemide. (FIG. 5A) shows a reaction scheme and screening conditions for engineering McbA for the enzymatic synthesis of moclobemide. (FIG. 5B) is a hot spot screen (HSS) of 64 identified residues in McbA showing percent conversion of moclobemide normalized to WT (n=1). Highly mutable sites are starred and included in additional engineering rounds. (FIGS. 5C, 5D). Iterative site saturation mutagenesis (ISM) of residues identified in the HSS, again showing normalized percent conversion of moclobemide. (FIG. 5E) depicts crystal structure of McbA (PDB: 6SQ8) with bound native substrates. Orange residues highlight the almost complete coverage of first shell residues in the active site explored in the HSS. (FIG. 5F) is a SDS-Page of purified McbA_{moc} variants expressed in e. coli BL21 (DE3) and purified using an N-terminal strep tag. The first lane is the lysate soluble fraction of the overexpressed protein and the second lane is the purified protein. The expected molecular weight was 55.5 kDa, and the purified protein lane was loaded with 10 μg of protein.

FIGS. 6A-6C. Assessing mutant's production of moclobemide. (FIG. 6A) is reversed-phase (RP)-HPLC traces of moclobemide product (red) and acid substrate (purple) of wt-McbA and engineered mutants. (FIG. 6B) is an additional ATP regeneration system sustains high percent conversions using low stoichiometric addition of AMP (n=3). (FIG. 6C) shows a preparative scale synthesis of moclobemide scaled 1000× from 10 μl to 10 ml, with 87% conversion of 58 mg in 10 ml sample; purified product pictured.

FIGS. 7A-7B. Michaelis-Menten graphs of McbA_{moc} variants. (FIG. 7A) depicts the coupled enzymatic reaction scheme to measure the Michaelis-Menten kinetics of the amine substrate (4-(2-aminoethyl)morpholine). (FIG. 7B). Michaelis-Menten graphs of all McbA_{moc} variants were plotted in GraphPad Prism and fit using the default Michaelis-Menten non-linear regression analysis tool.

FIG. 8. Melting temperatures of McbA_{moc} variants. Circular dichroism denaturation curves were min-max normalized for each sample for ease of comparison across mutants.

FIGS. 9A-9E. The diverse accessible chemical space of McbA suggests a biocatalyst capable of synthesizing several high value molecules. (FIG. 9A) depicts the reaction scheme and screening conditions for exploring the substrate scope of McbA for the enzymatic synthesis of amides. McbA was expressed using CFPS and the reaction was initiated by the addition of different combinations of acid and amine substrates. (FIG. 9B) illustrates the all-by-all substrate screen for McbA, analyzed with RP-HPLC (n=1). Darker red corresponds to a product that was observable by UV absorbance while lighter red corresponds to trace amounts only detectable by MSD. (FIG. 9C) shows among the 21 high value molecules that were possible in the substrate scope, McbA was able to synthesize 16 (11 of which are small-molecule pharmaceuticals). (FIG. 9D) are high value molecules that McbA was unable to synthesize under the tested reaction conditions. (FIG. 9E) shows the substrate scope of wt-McbA; all acids and amines tested and their CAS numbers are included.

FIGS. 10A-10D. The diverse accessible chemical space of McbA suggests a biocatalyst capable of synthesizing several high value molecules. (FIG. 10A) depicts the reaction scheme and screening conditions for exploring the substrate scope of McbA for the enzymatic synthesis of amides. McbA was expressed using CFPS and the reaction was initiated by the addition of different combinations of acid and amine substrates. (FIG. 10B) illustrates the all-by-all substrate screen for McbA, analyzed with RP-HPLC (n=1). Darker red corresponds to a product that was observable by UV absorbance while lighter red corresponds to trace amounts only detectable by MSD. (FIG. 10C) shows among the 21 high value molecules that were possible in the substrate scope, McbA was able to synthesize 16. (FIG. 10D) are high value molecules that McbA was unable to synthesize under the tested reaction conditions.

FIGS. 11A-11D. Engineering campaign for the current FDA-approved drug cinchocaine. (FIG. 11A) depicts the reaction scheme for the biosynthesis of cinchocaine using McbA. (FIG. 11B) illustrates HSS of 64 identified residues in McbA showing percent conversion of cinchocaine normalized to WT (n=1) as determined by absorbance on HPLC. Highly mutable sites are starred and included in additional engineering rounds. (FIG. 11C) shows after one additional engineering round, no additional beneficial mutation beyond the double mutant (pathway 1) was found. Mutations that were previously observed to be beneficial were no longer in future rounds. An additional engineering round using a double mutant consisting of the two best mutations found in the HSS (pathway 2) also did not show additional mutations for this backbone. (FIG. 11D) illustrates RP-HPLC trace of cinchocaine product (red) and acid substrate (purple) of wt-McbA and engineered mutants.

FIGS. 12A-12I. Machine learning-guided evolution predicts highly active mutants with a lower screening burden than iterative site saturation mutagenesis. (FIG. 12A) illustrates the strategy for applying machine learning to bypass iterative rounds of saturation mutagenesis. A supervised ridge regression model is trained on percent conversion data of four mutable sites selected from the HSS, with sequence features consisting of an amino acid encoding augmented with a zero-shot prediction of enzyme fitness. From a training set of approximately 80 single mutants, we extrapolate higher order mutations and test the top 25 predictions. (FIG. 12B, 12C) show the percent conversion (n=3) of ML-predictions for both moclobemide (FIG. 12B) and metoclopramide (FIG. 12C), with the best quadruple mutant from ISM (M4) colored grey. (FIG. 12D, 12E) show analysis of model fidelity with training sets built with smaller libraries than saturation mutagenesis, including reduced codon sets (NDT, NRT) and reduced amino acid alphabets based on BLOSUM50. Comparing measured vs. predicted activity on withheld ISM rounds is shown for models trained on the complete saturation mutagenesis dataset for both moclobemide (moc) (FIG. 12D), and metoclopramide (meto) (FIG. 12E). (FIGS. 12F-12I) show complete characterization of augmented ridge regression model performance using different combinations of fitness predictors and amino acid encodings. While NDCG was the selection criteria for model performance, the Spearman correlation coefficient was included to better explore the differences in all the models tested. In most instances, these two model performance indicators are closely aligned. Graphs display NDCG (FIG. 12F) and Spearman correlation (FIG. 12G) for moclobemide, and NDCG (FIG. 12H) and Spearman correlation (FIG. 12I) for metoclopramide.

FIGS. 13A-13D. Hot spot screen and machine-learning guided predictions for the biocatalytic synthesis of cinchocaine. (FIG. 13A) is the chemical structure of cinchocaine. (FIG. 13B) illustrates HSS of 64-site library to identify McbA residues that positively impact cinchocaine synthesis (n=1). Yields are normalized to wt-McbA. (FIG. 13C) is experimental validation of the top 24 ML-predictions, rank ordered. The best single mutant from the HSS (orange) and the best “rational” design from combining all the best mutations from the HSS (blue) were also included. (FIG. 13D) illustrates that machine-learning guided predictions have increased activity over wt-McbA for the synthesis of cinchocaine. Reversed-phase (RP)-HPLC traces of cinchocaine product (red) and acid substrate (purple) of wt-McbA (‘WT’) and the machine learning predicted mutant with the highest activity compared to an authentic standard.

FIGS. 14A-14D. Workflow for parallelized protein engineering. (FIG. 14A) illustrates a single protein can be used to produce distinct engineered biocatalysts. (FIG. 14B) depicts first, potential target reactions are identified; second, hot spot screening selects residues with significant impact on target reaction; and third, machine learning predictions are tested and used to guide future rounds of protein engineering. (FIG. 14C) is a comparison of the highest activity predicted variant for cinchocaine compared to wt-McbA and an authentic standard. Enzyme concentrations was normalized to 0.5 mg/mL (approximately 9 μM) and products were analyzed by LC-MS. The fold-increase in yield observed compares wt-McbA to ML-McbA (n=3). (FIG. 14D) shows the chemical structures of small-molecule pharmaceuticals used in engineering campaigns.

FIGS. 15A-15D. Hot spot screen and machine-learning guided predictions for the biocatalytic synthesis of itopride. (FIG. 15A) is the chemical structure of itopride. (FIG. 15B) illustrates HSS of 64-site library to identify McbA residues that positively impact itopride synthesis (n=1). Yields are normalized to wt-McbA. (FIG. 15C) illustrates experimental validation of the top 24 ML-predictions, rank ordered. As wt-McbA and several mutants only produce trace amounts only quantifiable by MSD, yield was measured by extracted ion chromatogram (EIC) (m/z of 359.2). The best single mutant from the HSS (orange) and the best “rational” design from combining all the best mutations from the HSS (blue) were also included. (FIG. 18D) is a graphic comparison of the highest activity predicted variant for itopride compared to wt-McbA and an authentic standard. Enzyme concentration was normalized to 0.5 mg/mL (approximately 9 μM) and products were analyzed by LC-MS. The fold-increase in yield observed compares wt-McbA to ML-McbA (n=3).

FIGS. 16A-16D. Hot spot screen and machine-learning guided predictions for the biocatalytic synthesis of declopramide. (FIG. 16A) is the chemical structure of declopramide. (FIG. 16B) illustrates HSS of 64-site library to identify McbA residues that positively impact declopramide synthesis (n=1). Yields are normalized to wt-McbA. (FIG. 18C) illustrates experimental validation of the top 24 ML-predictions, rank ordered. As wt-McbA and several mutants only produce trace amounts only quantifiable by MSD, yield was measured by EIC (m/z of 270.1). The best single mutant from the HSS (orange) and the best “rational” design from combining all the best mutations from the HSS (blue) were also included. (FIG. 18D) is a graphic comparison of the highest activity predicted variant for declopramide compared to wt-McbA and an authentic standard. Enzyme concentration was normalized to 0.5 mg/mL (approximately 9 μM) and products were analyzed by LC-MS. The fold-increase in yield observed compares wt-McbA to ML-McbA (n=3).

FIGS. 17A-17D. Hot spot screen and machine-learning guided predictions for the biocatalytic synthesis of trimethobenzamide. (FIG. 17A) is the chemical structure of trimethobenzamide. (FIG. 17B) illustrates the HSS of 64-site library to identify McbA residues that positively impact trimethobenzamide synthesis (n=1). Yields are normalized to wt-McbA. (FIG. 17C) illustrates experimental validation of the top 24 ML-predictions, rank ordered. As wt-McbA and several mutants only produce trace amounts only quantifiable by MSD, yield was measured by EIC (m/z of 389.2). The best single mutant from the HSS (orange) and the best “rational” design from combining all the best mutations from the HSS (blue) were also included. (FIG. 18D) is a graphic comparison of the highest activity predicted variant for trimethobenzamide compared to wt-McbA and an authentic standard. Enzyme concentration was normalized to 0.5 mg/mL (approximately 9 μM) and products were analyzed by LC-MS. The fold-increase in yield observed compares wt-McbA to ML-McbA (n=3).

FIGS. 18A-18D. Hot spot screen and machine-learning guided predictions for the biocatalytic synthesis of S-sulpiride. (FIG. 18A) is the chemical structure of S-sulpiride. (FIG. 18B) illustrates HSS of 64-site library to identify McbA residues that positively impact S-sulpiride synthesis (n=1). Yields are normalized to wt-McbA. (FIG. 18C) illustrates experimental validation of the top 24 ML-predictions, rank ordered. As wt-McbA and several mutants only produce trace amounts only quantifiable by MSD, yield was measured by EIC (m/z of 342.1). The best single mutant from the HSS (orange) and the best “rational” design from combining all the best mutations from the HSS (blue) were also included. (FIG. 18D) is a graphic comparison of the highest activity predicted variant for S-sulpiride compared to wt-McbA and an authentic standard. Enzyme concentration was normalized to 0.5 mg/mL (approximately 9 μM) and products were analyzed by LC-MS. The fold-increase in yield observed compares wt-McbA to ML-McbA (n=3).

FIGS. 19A-19D. Hot spot screen and machine-learning guided predictions for the biocatalytic synthesis of procainamide. (FIG. 19A) is the chemical structure of procainamide. (FIG. 19B) illustrates HSS of 64-site library to identify McbA residues that positively impact procainamide synthesis (n=1). Yields are normalized to wt-McbA. (FIG. 19C) illustrates experimental validation of the top 24 ML-predictions, rank ordered. As wt-McbA and several mutants only produce trace amounts only quantifiable by MSD, yield was measured by EIC (m/z of 236.1). The best single mutant from the HSS (orange) and the best “rational” design from combining all the best mutations from the HSS (blue) were also included. (FIG. 19D) is a graphic comparison of the highest activity predicted variant for procainamide compared to wt-McbA and an authentic standard. Enzyme concentration was normalized to 0.5 mg/mL (approximately 9 μM) and products were analyzed by LC-MS. The fold-increase in yield observed compares wt-McbA to ML-McbA (n=3).

FIGS. 20A-20D. Hot spot screen and machine-learning guided predictions for the biocatalytic synthesis of troxipide. (FIG. 20A) is the chemical structure of troxipide. (FIG. 20B) illustrates HSS of 64-site library to identify McbA residues that positively impact troxipide synthesis (n=1). Yields are normalized to wt-McbA. (FIG. 20C) illustrates experimental validation of the top 24 ML-predictions, rank ordered. As wt-McbA and several mutants only produce trace amounts only quantifiable by MSD, yield was measured by EIC (m/z of 295.1). The best single mutant from the HSS (orange) and the best “rational” design from combining all the best mutations from the HSS (blue) were also included. (FIG. 20D) is a graphic comparison of the highest activity predicted variant for troxipide compared to wt-McbA and an authentic standard. Enzyme concentration was normalized to 0.5 mg/mL (approximately 9 μM) and products were analyzed by LC-MS. The fold-increase in yield observed compares wt-McbA to ML-McbA (n=3).

FIGS. 21A-21B shows the two-step reaction mechanism of McbA allows probing the impact of mutations on the different reaction steps. (FIG. 21A) shows the concerted reaction mechanism of ATP-dependent amide bond synthetases, such as McbA, is first the activation of an acid to an acyl-adenylate intermediated followed by substitution with an amine nucleophile. This example focuses on the synthesis of procainamide (red) using 4-aminobenzoic acid (purple) and N,N-diethylethylenediamine (blue). (FIG. 21B) shows that since the acyl-adenylate intermediate (orange) is also visible by LCMS, how different mutations affect the acid adenylation step vs. the amide bond forming step are compared. The MSD trace (EIC with m/z of 236.1) is also included due to the low signal of procainamide under UV absorbance. Key: ‘ML’ is best ML-predicted mutant, ‘ISM’ is the best ‘rational’ design from combining the best mutations from the HSS, and ‘HSS’ is the best single mutant from the HSS.

FIGS. 22A-22B. Modeling the active sites of the best ML-predicted mutations reveals trends in mutations for different substrates. For each of the seven compounds that had the machine-learning guided framework to engineer applied, (FIG. 22A) cinchocaine, declopramide, itopride, and trimethobenzamide, and (FIG. 22B) sulpiride, procainamide, and troxipide, the active site was visualized to infer why certain mutations are prevalent among different substrates. The previously crystalized McbA (PDB: 6SQ8) was used as the backbone with the native substrates in the active site. Amino acid changes were made at select residues using the rotamer tool with default parameters in ChimeraX.

FIG. 23A-23C. Cell-free enzyme engineering of a formyl-CoA synthetase.

FIG. 24. Single site saturation mutagenesis round for engineering EryACS.

FIG. 25. Evolution of Erythobacter sp. acyl-CoA synthetase towards activity with formate. Engineering campaign 1 resulted in a ACS that is highly selective for formate over acetate than the WT (˜14,000 fold increase in specificity)

FIG. 26. A second directed evolution of Erythobacter sp. acyl-CoA synthetase towards activity with formate. Engineering campaign 2 resulted in an ACS that is more active for formate than the WT (˜10 fold increase in activity).

FIGS. 27A-27D. Illustratrations of formyl-CoA synthetase evolution.

FIG. 28. Block diagram of an example cell-free protein engineering system in accordance with some examples described in the present disclosure.

FIG. 29. Block diagram of example components that can implement the system of FIG. 28.

### DETAILED DESCRIPTION

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In aspects, the present methods can be implemented as a computer program product that comprises a computer program mechanism embedded in a non-transitory computer readable storage medium. These program modules can be stored on a CD-ROM, DVD, magnetic disk storage product, USB key, or any other non-transitory computer readable data or program storage product.

The disclosed subject matter may be implemented as a system, method, apparatus, or article of manufacture using programming and/or engineering techniques to produce software, firmware, hardware, or any combination thereof to control a computer or processor based device to implement aspects detailed herein. The term “article of manufacture” (or alternatively, “computer program product”) as used herein is intended to encompass a computer program accessible from any computer-readable device, carrier, or media. For example, computer readable media can include but are not limited to magnetic storage devices (such as hard disk, floppy disk, magnetic strips), optical disks (such as compact disk (CD), digital versatile disk (DVD)), smart cards, and flash memory devices (such as card, stick). Additionally, it should be appreciated that a carrier wave can be employed to carry computer-readable electronic data such as those used in transmitting and receiving electronic mail or in accessing a network such as the Internet or a local area network (LAN). Transitory computer-readable media (carrier wave and signal based) should be considered separately from non-transitory computer-readable media such as those described above. Of course, those skilled in the art will recognize many modifications may be made to this configuration without departing from the scope or spirit of the claimed subject matter.

### Exemplary Applications

Disclosed herein are methods, systems, and evolved catalysts (protein or enzyme catalysts), directed to iterative saturation mutagenesis (ISM) and cell-free variant protein (enzyme) synthesis. The disclosed methods and systems have improved efficiency and high-throughput function. Derived variant or mutant enzymes have desired functionality. In some embodiments, disclosed methods comprise a completely in vitro iterative site saturation mutagenesis workflow centered around cell-free protein synthesis (CFPS) that is capable of library generation and expression in less than 24 hours. The workflow comprises iterating through several steps carried out in a multiplex platform (e.g. 384-well plates or similar platform) using liquid handling robots: 1) in-vitro DNA assembly, 2) cell-free enzyme expression, 3) high-throughput activity assay.

Evolved variant enzymes showcase the utility of the disclosed methods and systems. Exemplary variant enzymes were evolved that catalyze two distinct chemical transformations. First, an acyl-CoA synthetase was evolved for the activation of formate to formyl-CoA, a key reaction in synthetic carbon fixation pathways. Second, an amide synthetase was evolved to generate a panel of compounds with pharmaceutical relevance. The usefulness of the disclosed methods demonstrates application to any enzyme of interest and identifies several important enzymes for defined applications.

The disclosed technologies may be applied to exemplary areas: high-throughput enzyme directed evolution, biochemical upgrading of formate into value-added chemicals, and sustainable amide bond formation & biopharmaceutical production.

The disclosed technologies can be used to perform parallel engineering campaigns which allows faster and easier development of new proteins. The platform can serve as a blueprint for the maturation of biocatalysts to enable efficient, economical, and environmentally benign processes for biomanufacturing.

### Enzyme Engineering

Many important biotransformations require efficient enzymes that have yet to be discovered or engineered. High-throughput methods to not only study enzymes but evaluate their engineering potential as industrial tools are crucial. A cell-free DNA assembly and protein synthesis platform is developed, allowing rapid design and screening of thousands of sequence-defined enzyme mutants in days. These rich datasets effectively map sequence-fitness landscapes that are well suited for machine learning applications. The simplicity, speed, and versatility of this method allow a single user to effectively engineer an enzyme for multiple functions simultaneously, shifting the focus from single molecule targets to multiple distinct regions of chemical space. This method is showcased by performing the parallelized protein engineering of an amide synthetase for nine small-molecule pharmaceuticals. This platform will serve as a blueprint for the maturation of biocatalysts to enable efficient, economical, and environmentally benign processes for biomanufacturing.

Enzymes play a vital role in realizing a sustainable bioeconomy and tackling critical global challenges by offering green, programmable, and efficient solutions to traditional chemical processes. For example, engineered enzymes are being used to upgrade carbon waste into value-added chemicals¹, degrade plastics², and produce blockbuster pharmaceuticals³. Many of these examples have been derived or inspired by naturally existing enzymes⁴. However, leveraging the natural plasticity and promiscuity of enzymes to enable these advances is incredibly challenging due to our inability to connect amino acid sequences with desired functions. One solution is to overcome this challenge by using directed evolution, making use of enzyme mutant libraries and experimental screening or selections to obtain an enzyme with a desired function^{5,6}. With selection methods focusing on “winning” enzymes for one particular transformation, these approaches are limited in their ability to collect sequence-function relationships for further engineering of similar reactions. Additionally, screening methods are limited to experimentally testing hundreds to thousands of unique mutants and often miss out on epistatic interactions among different residues^{7,8}. This at best allows for focusing on improving a single unique transformation or at worst may not yield a useful enzyme. New tools that increase our ability to screen enzyme mutants and collect and leverage sequence-function data could enable protein engineering campaigns that solve a more general reaction optimization problem and yield sets of enzymes and amino acid residues that can be modified for many desired reactions.

Machine learning-based strategies are increasingly being leveraged to overcome these challenges by moving much of the screening burden from experimental to computational^{9,10}. Among these, there have been two main approaches employed to engineer enzymes. The first is to train a supervised regression model on assayed fitness data, where amino acid sequence featurization ranges from simple site-specific one-hot encodings to complex whole-sequence nonlinear transformations¹¹⁻¹⁵, and then use these models to predict protein variants enriched in a desired function¹⁶⁻¹⁸. Typically, variant composition is restricted to a few residues (e.g., four positions¹⁹) and mutated away from a single target sequence. The second approach is to train unsupervised models on related homologs²⁰⁻²³ or a large set of unlabeled sequences²⁴ to infer the fitness of a protein variant relative to wild type (a so-called zero-shot prediction). Even though measured fitness values are not incorporated, the occurrence of the protein in nature implies it passed evolutionary constraints. Several groups have shown the predictive power of combining these approaches^{12,25,26}. Regardless of model architecture employed, building enriched datasets is key to train these models and successfully navigate vast sequence spaces quickly¹⁶. An end-to-end generalizable strategy for reaction class-focused enzyme engineering that takes advantage of these advances in machine learning is still an open challenge.

One exemplary reaction type that poses a general reaction optimization problem is the formation of amide bonds—a motif ubiquitously found in pharmaceuticals, agrochemicals, polymers, fragrances, flavors, non-lethal munitions, and other high-value products²⁷. Contemporary chemical methods for amide formation are remarkably general but widely regarded as expensive, inelegant, and unsustainable²⁸⁻³⁰. Luckily, nature has evolved a range of enzymes to construct amide bonds with unique advantages over their synthetic counterparts such as chemo-, stereo-, and regioselectivities and displaying impressive activity under mild reaction conditions (e.g., aqueous, atmospheric pressure and temperature)³¹⁻³³. A recently discovered group of enzymes, called amide bond synthetases (ABSs), offers new and underexplored promise for biocatalytic amide bond formation as their reaction chemistry is comparatively simple and the chemical space they can access is closely related to molecules of pharmaceutical interest. One such enzyme is McbA from Marinactinospora thermotolerans, an ATP-dependent ABS involved in the biosynthesis of marinacarboline secondary metabolites³⁴. A few studies have explored the permissible substrate scope of McbA, utilizing simple acids and amines commonly found in pharmaceuticals, which suggests McbA could serve as a flexible starting point for enzyme engineering^{35,36}. Distinct transformations require distinct enzymes and therefore, while the potential engineering space of McbA is large, it is difficult to reach by methods that focus on single transformations. Given the value of developing efficient biocatalysts for amide bond formation, McbA and ABSs are important targets for enzyme engineering efforts.

Combining aspects of directed evolution, machine learning, and cell-free synthetic biology, an in vitro, high-throughput, and rapid protein engineering workflow has been developed for map sequence-reaction class relationships by screening a large single-mutant library against multiple substrates to determine mutations that alter substrate preference. These data are then fit to ridge regression models augmented with a variety of zero-shot predictors and these models are used to extrapolate higher-order mutants with increased activity. Collectively, the parallelized protein engineering efforts on McbA generated a set of nine distinct enzymes for the biosynthesis of nine small molecule pharmaceuticals. While the focus was on amide synthetases here, this approach could be easily adapted to engineer a broad range of enzymes.

### Methods of Cell-Free Directed Evolution

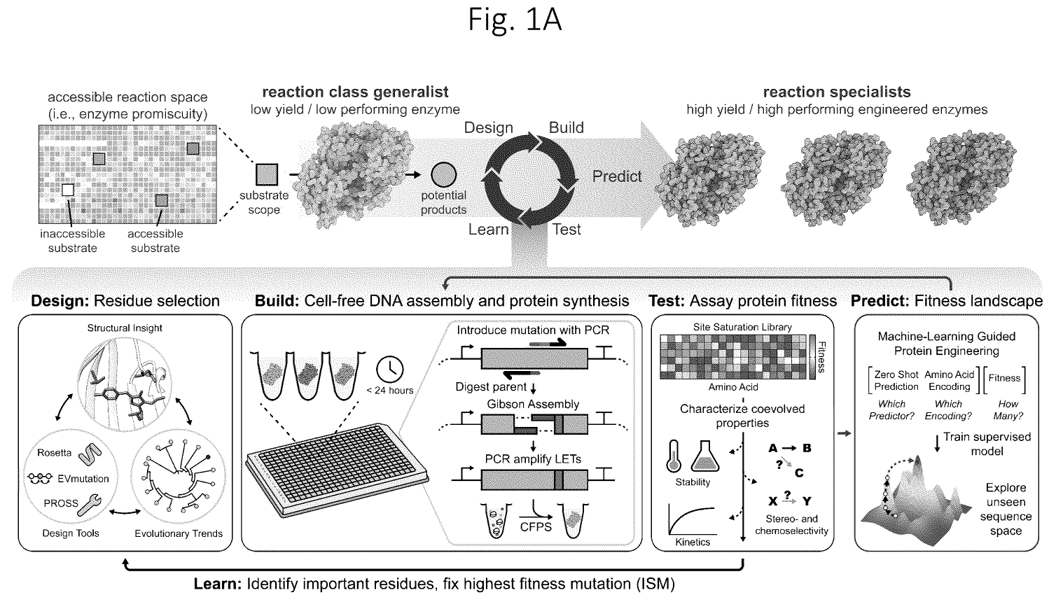

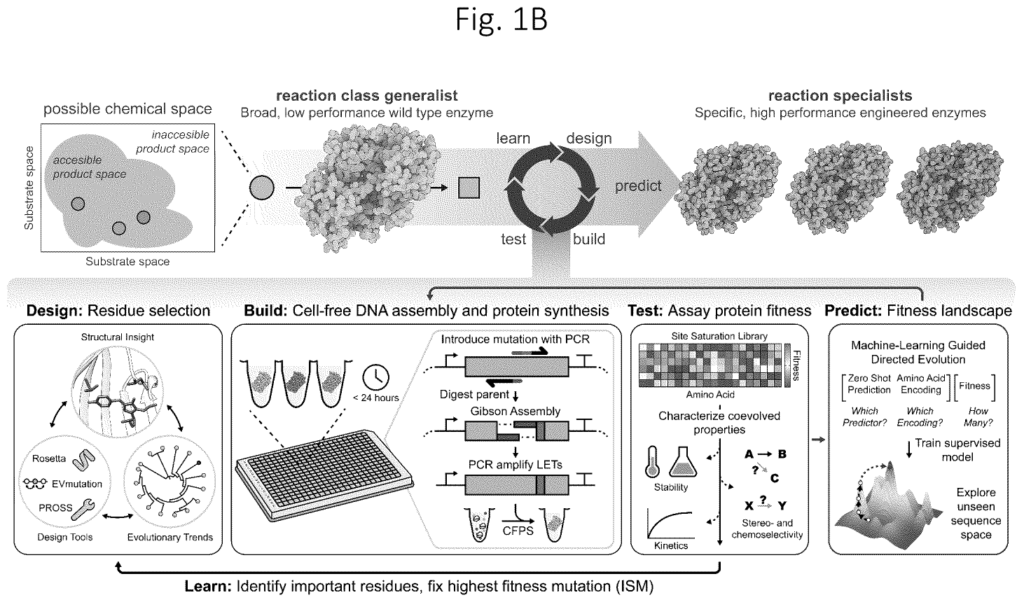

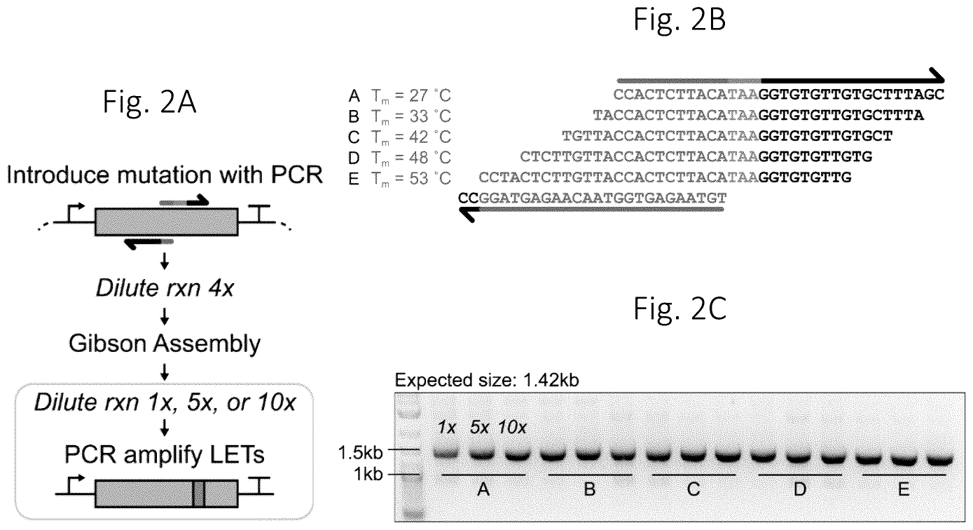

For convenience and clarity, the methods disclosed herein are detailed in four phases. An overview of the phases is presented in FIGS. 1A-1B. In Phase 1, potentially beneficial mutations are identified and created (see, FIGS. 2A-2C). Desired mutations are identified using hot spot mutations, and these desired mutations are introduced via PCR and Gibson assembly. The expression of these mutated DNA sequences is then amplified using PCR, and CFPS is used to express the mutated genes. For an overview of Phase 1, see FIGS. 2A-2C.

In Phase 2, the function of the mutants is assessed and quantified. The methods of Phase 2 vary based on how the mutant is being evaluated. This can range from quantifying the relative intensity of a mutated fluorescent protein, to activity assays or analytics to detect different products. An illustrative example of identifying key residues, using Phase 1 to produce mutations of those residues, and Phase 2 to assess the impact of those mutations is shown in FIGS. 2A-2C, using mutated muGFP as an example.

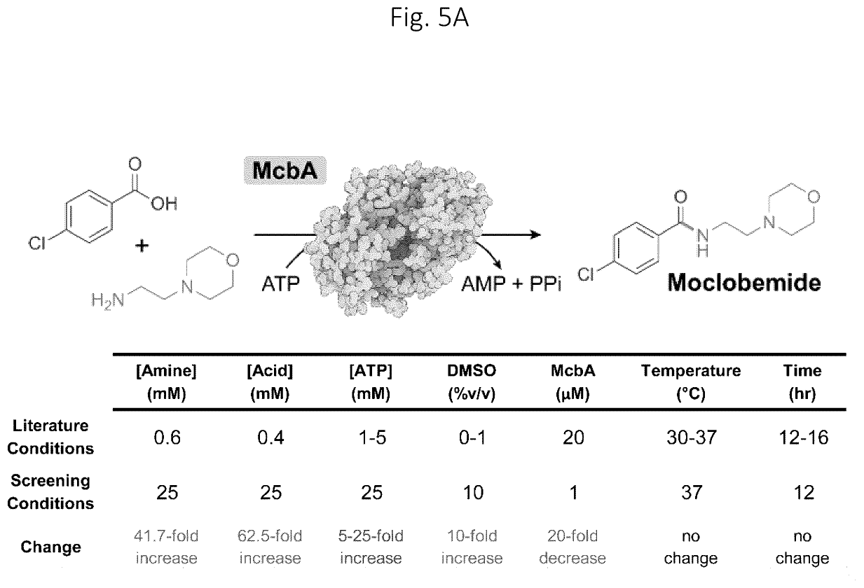

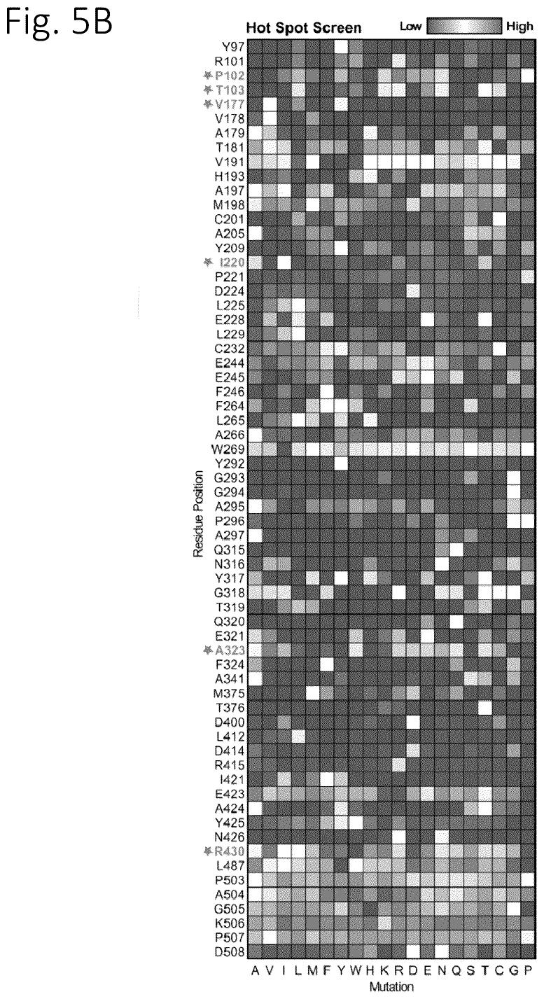

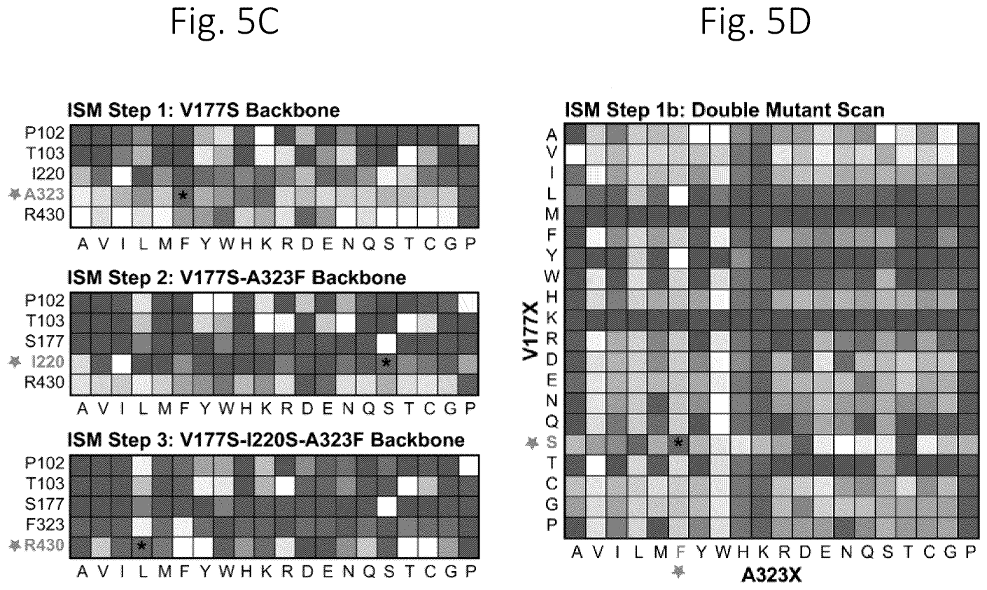

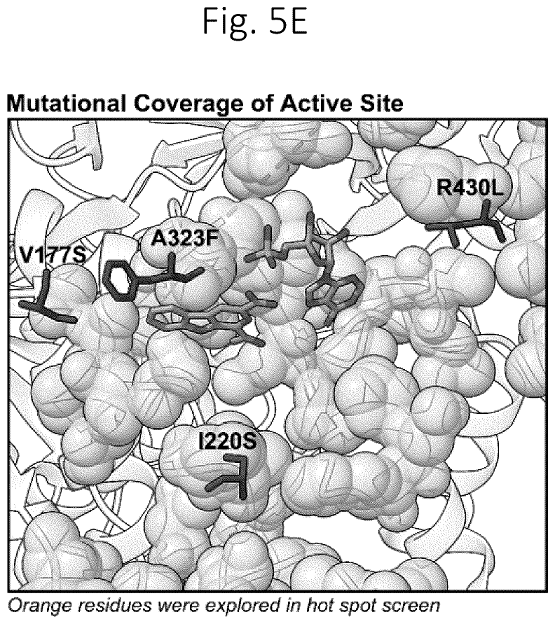

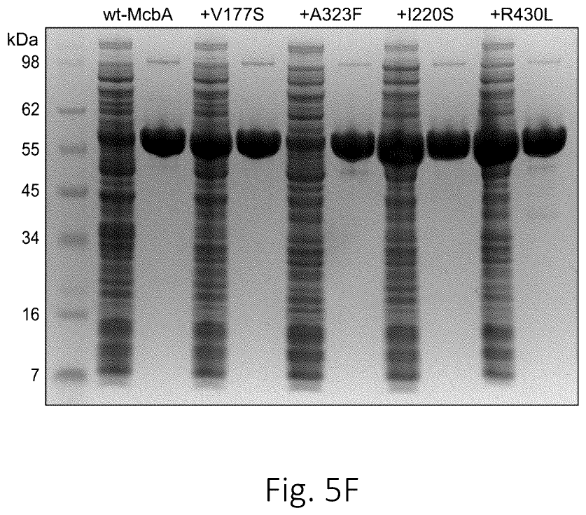

In Phase 3, ISM is used to accumulate beneficial combinations of mutations based on impactful residue locations identified in Phases 1 and 2. Essentially, the best performing mutants from Phase 1, as assessed by Phase 2, are used to test different combinations of mutations. This allows one to create multiple mutations in the same sequence and further assess to determine the best performing mutated gene. An example iterating through Phases 1-3 to identify beneficial combinations of mutations is shown in FIG. 5.

Phase 4 utilizes machine learning to aid in directed evolution. In one or more aspects, a correlation may be determined and/or the attributes of a particular residue mutation may be determined using one or more algorithms, including machine learning algorithms or models, executed on one or more computing devices having a processor and memory. In various aspects, the attributes associated with a particular amino acid sequence that are used in the above correlation determinations may be generated via a machine learning-based process. For example, structural information on the sequence may be inputted into a database or program that allows a machine learning algorithm or model to predict changes unfolding free energy in response to different mutations.

In some aspects, extracting a combination of, or all of the discrete features of the amino acid sequence parameters and characteristics is one way of obtaining a comprehensive discrete representation from distributional statistics. In certain aspects, these features can form feature vectors that are then utilized to facilitate training of machine learning algorithms or models for tasks such as predicting which residue mutations will have a significant impact on the desired reaction.

In aspects, in addition to extracting features in all aforementioned cases, to increase the accuracy of a trained machine learning algorithm or model, a plurality of data pre-processing methods for standardization, outlier detection, and anomaly detection may be incorporated into the feature space. Given the pre-processed data, the desired downstream task can be achieved via different approaches.

In some embodiments, an amino acid sequence of a wildtype protein can be used as input for machine learning algorithms or models. In other embodiments, single mutants derived from the hot spot screening (HSS) that were identified as having beneficial effects in Phase 2 can be used as inputs. The machine learning algorithms or models can then be used to extrapolate higher order mutants with increased beneficial effects.

Utilizing machine learning algorithms or models offers several advantages. Integrating machine learning can overcome path dependencies, reduce screening burden, and aid in taking larger steps in sequence space. Using mutants identified as beneficial in Phase 2 as inputs for machine learning algorithms further reduces the screening burden. Machine learning algorithms or models help identify potentially beneficial residue selections that would be missed by traditional computation methods. Furthermore, machine learning algorithms significantly speed up the rate at which residue selection can take place.

The outputs of the machine learning algorithms or models can then be used as residue selection when repeating Phase 1 in subsequent cycles. This method iterates through the phases to identify the combination of mutations that produces the best performance. It is entirely in vitro and can be completed in under 24 hours, which is significantly faster than previous methods. Furthermore, it allows one to study combinations of mutations at different residues rather than a single residue. The use of machine learning in Phase 4 can help identify which residues have significance for function traditional methods miss.

### Systems

A system useful for generating the evolved enzymes is provided. An aspect is a system that includes a computer system, a DNA expression template, and cell-free expression reagents.

A computer system may have one or more processors, and memory storing one or more programs for execution by the one or more processors.

The DNA expression templates have nucleic acid sequences encoding proteins. In embodiments, the DNA expression templates have nucleic acid sequences encoding variant proteins. The variant proteins are based on a reference protein. In the embodiments, the proteins comprise at least one variant amino acid residue as compared to the reference protein.

Cell-free expression reagents may include a cell lysate, DNA template, DNA-dependent RNA polymerase, a ribosome, dNTPs, tRNAs, NTPs, NMPs, secondary energy substrates (e.g., phosphorylated (PEP, acetyl-P, 3-PG), or non-phosphorylated energy compounds (glucose, pyruvate, glutamate, maltodextrin)), magnesium, salts, cofactors (coA, NAD), etc.

An embodiment includes a system comprising a computer system having one or more processors, and memory storing one or more programs for execution by the one or more processors; DNA expression templates comprising nucleic acid sequences encoding variant proteins, wherein the variant proteins are based on a reference protein and wherein the proteins comprise at least one variant amino acid residue as compared to the reference protein; cell-free expression reagents comprising a DNA-dependent RNA polymerase, ribosome, dNTPs, charged tRNAs, and ATP.

Another embodiment is a method for cell-free protein engineering using the computer system. The method includes using the computer system in multiple steps of the engineering scheme, including to select one or more mutations of a protein; assess the plurality of mutations of the protein; identify residue locations based on the assessed plurality of mutations of the protein; access a machine learning model; input DNA template data associated with the residue locations to the machine learning model; generating predicted site selection data as an output; and outputting the predicted site selection data to a user by the computer system.

In the embodiments, the machine learning model has been trained on training data to generate data that predict site selection for directed evolution of a protein. In the embodiments, the predicted site selection data predict residue locations having a selected effect when mutated.

### Novel Variant or Modified Enzymes and Methods of Using the Enzymes

In some embodiments, variant proteins or enzymes are evolved from the methods of cell-free evolution described herein. The evolved enzymes exhibit a function different from the wild-type counterpart. By way of example, but not by way of limitation, exemplary proteins are described below.

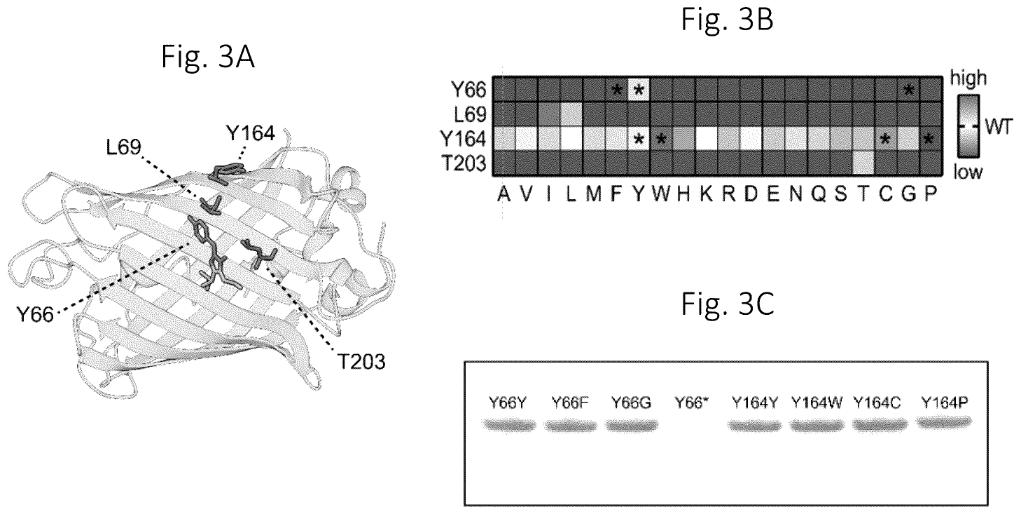

An exemplary protein is monomeric ultra-stable green fluorescent protein 40 (muGFP). An exemplary mutagenesis scheme is shown in FIGS. 3A-3C. Four potentially beneficial residues were identified using hot spot screening, as shown in FIG. 3A. These mutations were introduced using Phase 1 of the method described above. In Phase 2, the relative fluorescent intensity of each mutation was measured, shown in FIG. 3B. Top performing candidates were identified as a single mutation at residue Y164 (FIG. 3C).

Another exemplary protein is the amide bond synthetase enzyme McbA. McbA was mutated to improve the synthesis of monoamine oxidase A inhibitor, for example moclobemide (reaction shown in FIG. 5A). FIG. 5B shows the 64 residues identified by hot spot screening in Phase 1. These residues were assessed using Phase 1 and Phase 2 of the method, and the performance based on Phase 2 is shown in FIG. 5B. The top performing mutation, V177S, was then used in further rounds of the disclosed evolution method. Phases 1-3 were repeated for three consecutive rounds until the mutant was identified, (FIGS. 5C and 5D). These residues are shown in FIG. 5E.

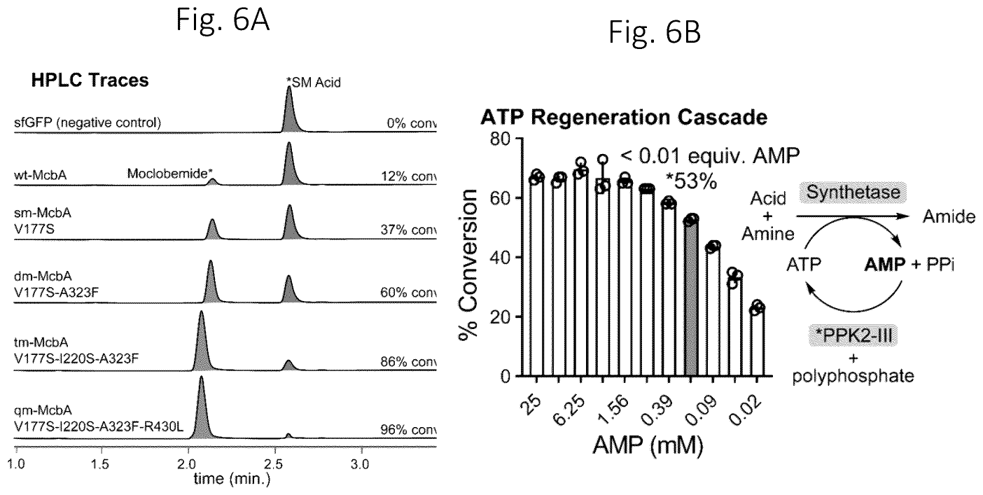

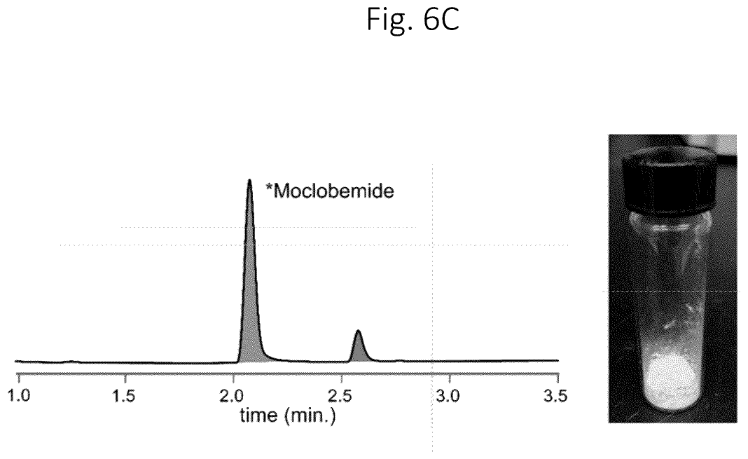

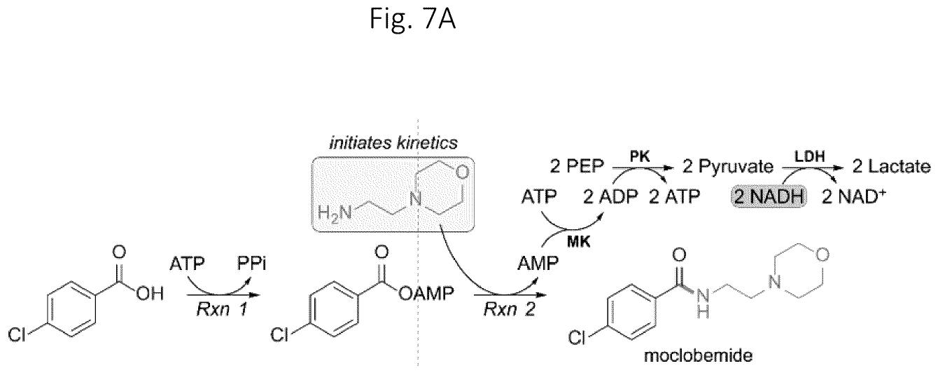

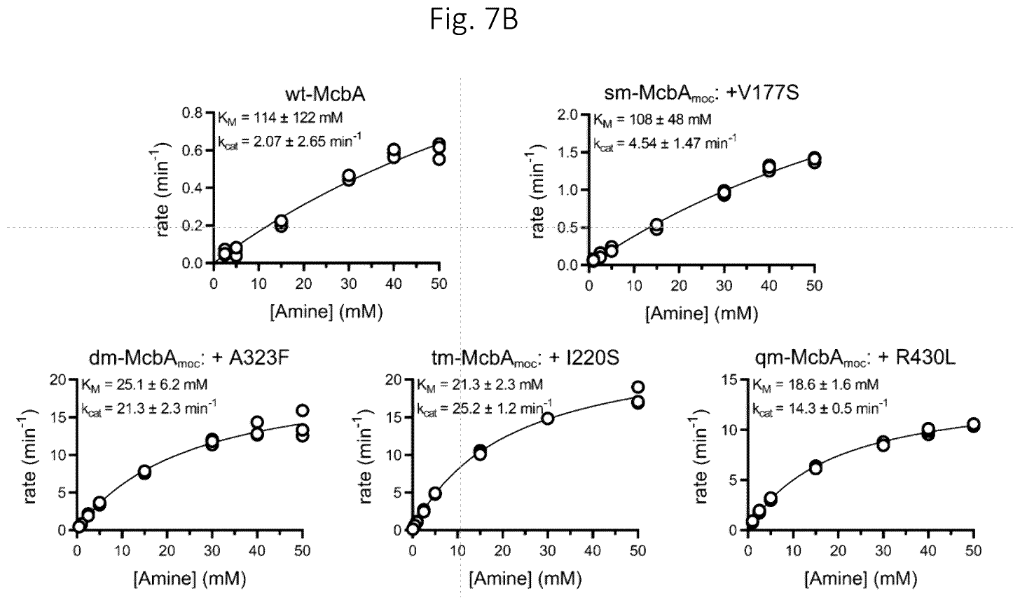

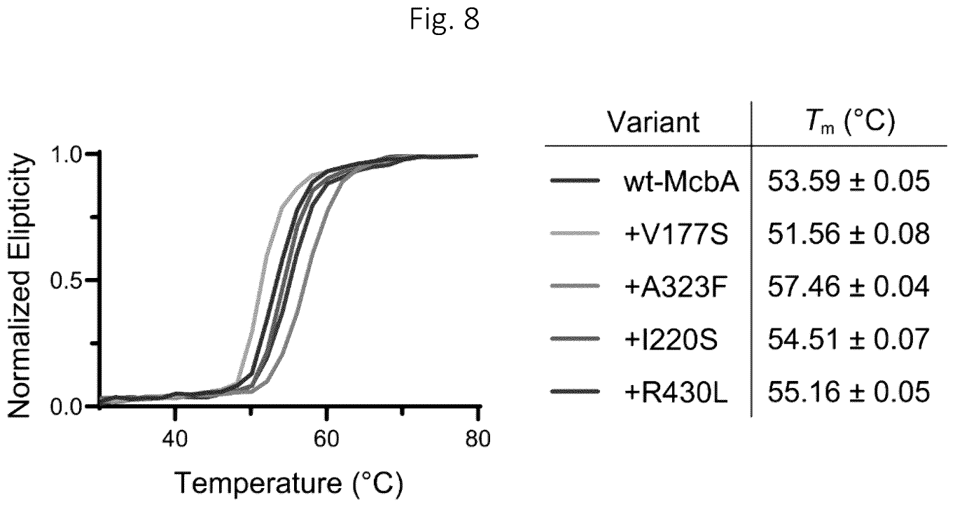

The performance of the top four McbA engineered mutations are shown in FIGS. 6A-6D. In FIG. 6A, the relative yield of moclobemide is shown for the negative control, wild type gene, and four top performing mutants. The reaction rate of each of the top mutants is shown in FIG. 7B. The melting temperature of each mutant is shown in FIG. 8.

The best performing McbA mutants were used to test the ATP recycling module by substituting various amounts of ATP with AMP (FIG. 6B). Then the reaction was scaled up 1,000 fold from 10 μl to 10 ml, and demonstrated that 58 mg for moclobemide was attained from a 10 mL sample, which showed an 87% conversion (FIGS. 6C-6D).

Next, eight protein engineering campaigns were completed for the amide synthetase of McbA to produce eight different products: metoclopramide, cinchocaine, itopride, declopramide, trimethobenzamide, (S)-sulupiride, procainamide, and troxipide. Each product has a distinct pharmaceutical use. The method described herein was used to find the best combination of mutations to enhance the production of each compound.

An embodiment comprising a modified amide synthetase (McbA) is provided. A reference amide synthetase was used to evolve the disclosed variant enzymes, with an amino acid sequence identified in SEQ ID NO: 2:

(SEQ ID NO: 2)
MEKKIWSHPQFEKGGSGENLYFQGGYARRVMDGIGEVAVTGAGGSVTGA
RLRHQVRLLAHALTEAGIPPGRGVACLHANTWRAIALRLAVQAIGCHYV
GLRPTAAVTEQARAIAAADSAALVFEPSVEARAADLLERVSVPVVLSLG
PTSRGRDILAASVPEGTPLRYREHPEGIAVVAFTSGTTGTPKGVAHSST
AMSACVDAAVSMYGRGPWRFLIPIPLSDLGGELAQCTLATGGTVVLLEE
FQPDAVLEAIERERATHVFLAPNWLYQLAEHPALPRSDLSSLRRVVYGG
APAVPSRVAAARERMGAVLMQNYGTQEAAFIAALTPDDHARRELLTAVG
RPLPHVEVEIRDDSGGTLPRGAVGEVWVRSPMTMSGYWRDPERTAQVLS
GGWLRTGDVGTFDEDGHLHLTDRLQDIIIVEAYNVYSRRVEHVLTEHPD
VRAAAVVGVPDPDSGEAVCAAVVVADGADPDPEHLRALVRDHLGDLHVP
RRVEFVRSIPVTPAGKPDKVKVRTWFTD.

In embodiments, a modified McbA has a sequence with one or more amino acid substitutions at amino acid residue Y97, R101, P102, T103, V177, V178, A179, T181, V191, H193, A197, M198, C201, A205, Y209, I220, P221, D224, L225, E228, L229, C232, E244, E245, F246, F264, L265, A266, W269, V292, G293, G294, A295, P296, A297, Q315, N316, Y317, G318, T319, Q320, E321, A323, F324, A341, M375, T376, D400, L412, D414, R415, I421, E423, A424, Y425, N426, R430, L487, P503, A504, G505, K506, P507, or 508 with reference to SEQ ID NO: 2.

In some embodiments, the modified McbA has one or more amino acid substitution is selected from a mutant amino acid residue, with reference to SEQ ID NO: 2, at position P102, T103, V177, I220, C232, A266, A323, or R430, or a homologue thereof.

In some embodiments, the modified McbA has one or more amino acid substitution is selected from a mutant amino acid residue, with reference to SEQ ID NO: 2, at position P102, T103, V177, C201, A205, C232, A424, R430, or a homologue thereof.

In some embodiments, the modified McbA has one or more amino acid substitution is selected from a mutant amino acid residue, with reference to SEQ ID NO: 2, at position P102, T103, V177, L225, A266, G318, T319, Q320, A323, I421, A424, R430, or D508, or a homologue thereof.

In some embodiments, the modified McbA has one or more amino acid substitution is selected from a mutant amino acid residue, with reference to SEQ ID NO: 2, at position P102, T103, V177, C201, I220, P221, L225, E228, C232, E244, A266, N316, T319, A323, E423, Y425, or R430, or a homologue thereof.

In some embodiments, the modified McbA has one or more amino acid substitution is selected from a mutant amino acid residue, with reference to SEQ ID NO: 2, at position P102, T103, V177, C201, A205, I220, P221, L225, C232, E244, A266, G318, T319, Q320, A323, E423, A424, Y425, or R430, or a homologue thereof.

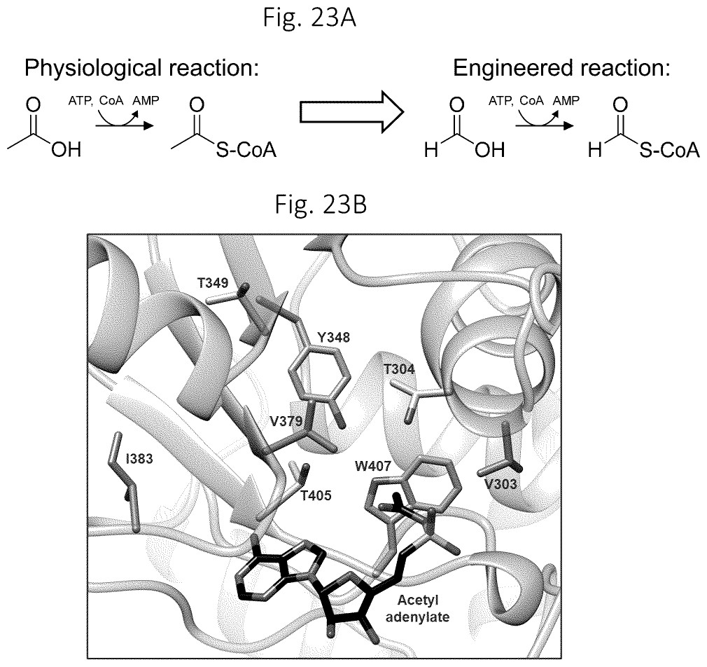

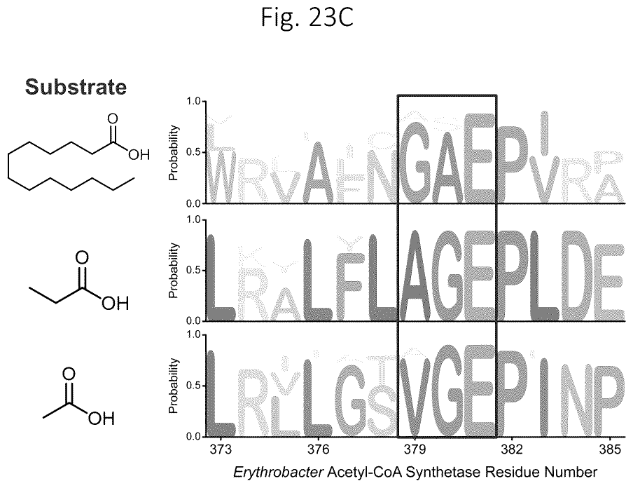

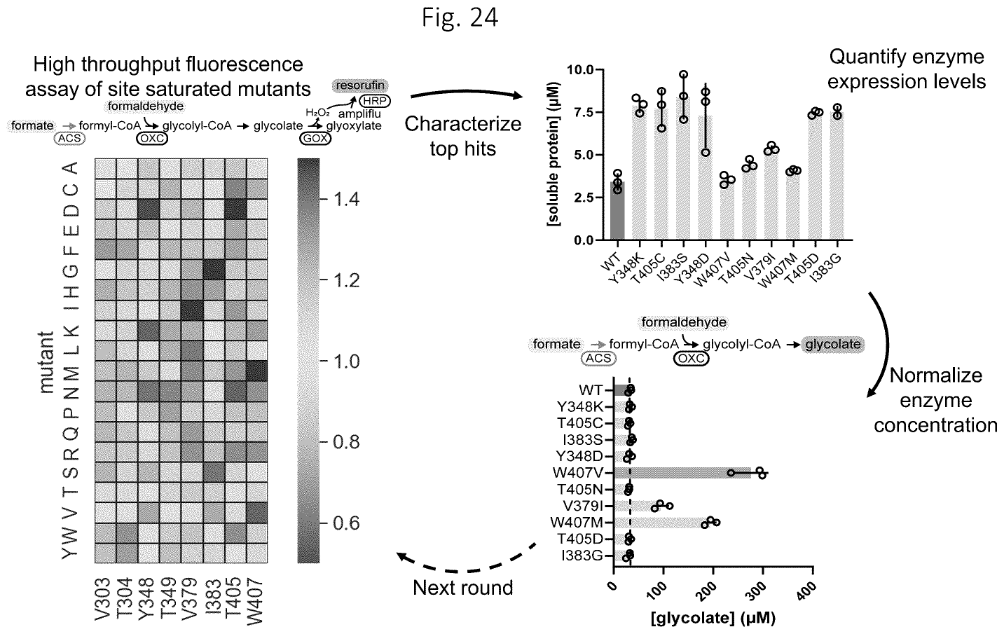

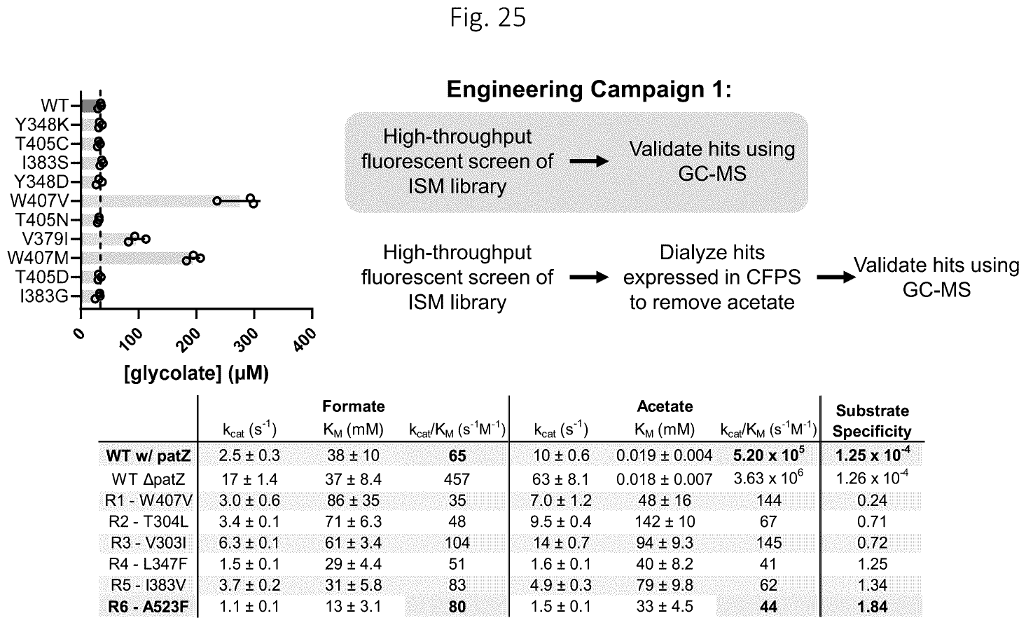

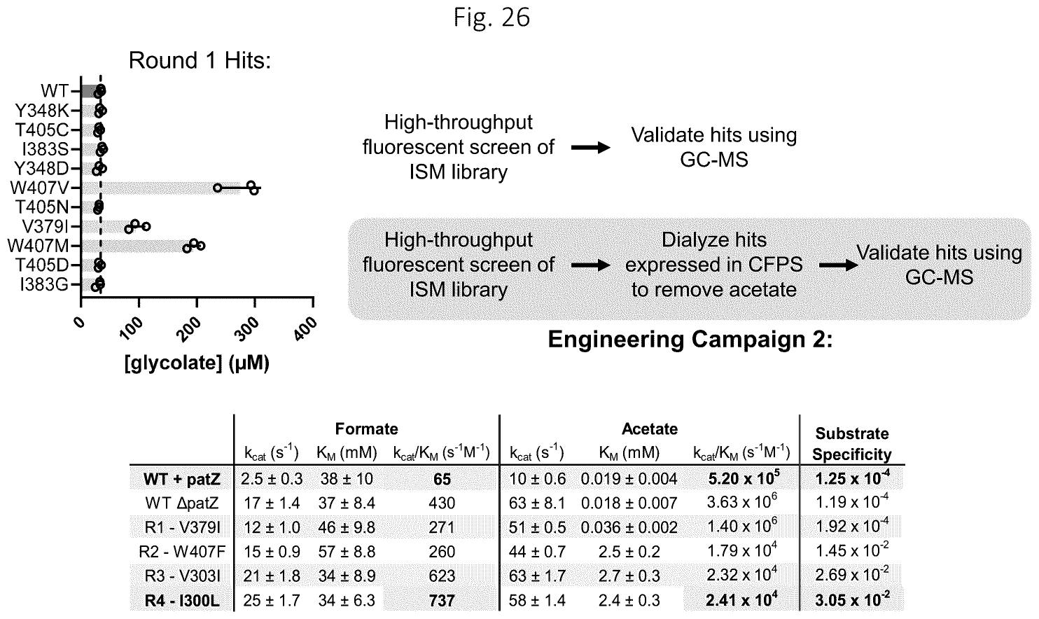

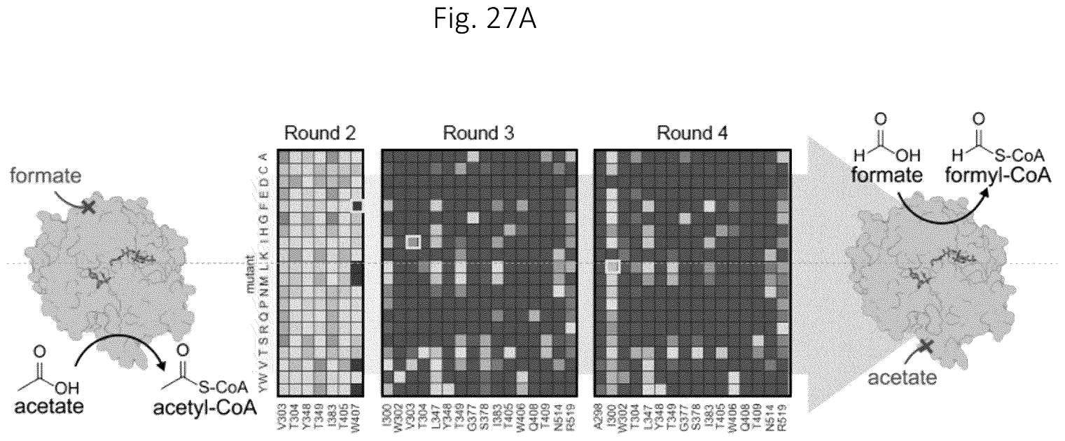

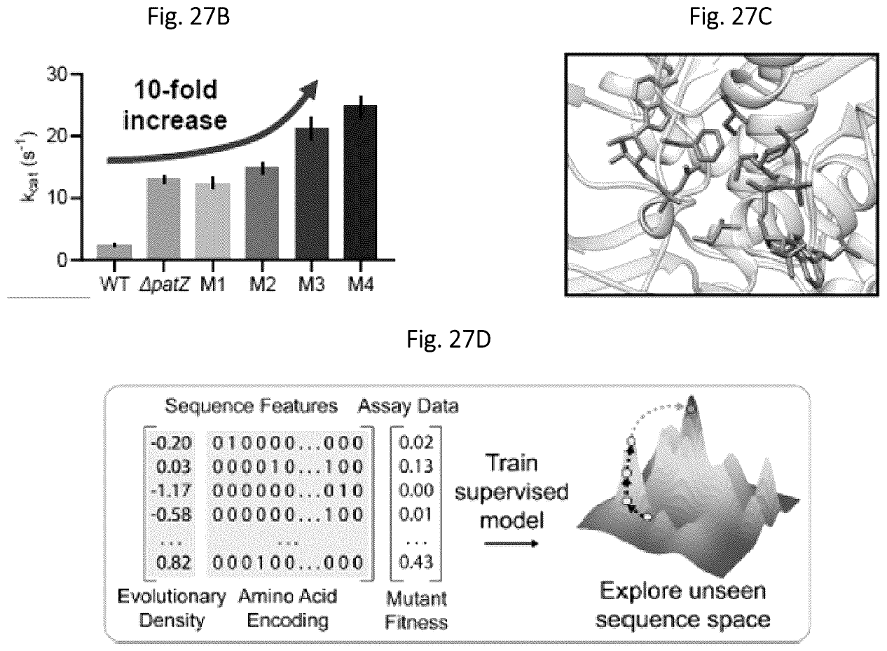

Another exemplary protein is acyl-CoA synthetase, which was evolved for the activation of formate to formyl-CoA, a key reaction in synthetic carbon fixation pathways. Some acyl-CoA synthetases possess the ability to catalyze the formation of acetate to acetyl-CoA. While formate is not a natural substrate of any currently known acyl-CoA synthetase, the promiscuous nature of enzymes allowed engineering of this unnatural activity (reactions shown in FIG. 23A). FIG. 23B shows residues that were selected in Phase 1, with the rationale of selecting putative residues that contribute to substrate specificity of the enzyme shown in FIG. 23C. These residues were assessed using Phase 1 and Phase 2 of the method, and the performance based on Phase 2 is shown in FIG. 24. Two top performing mutations were identified (W407V and V379I) and used for consecutive rounds in Phase 3 to accumulate mutations (6 and 4, respectively). Our evolution campaigns for these two starting top performing mutations revealed distinct characteristics. Starting with W407V (FIG. 25) resulted in an enzyme that had high specificity for formate over acetate but did not have improvements in activity. Conversely, starting with V379I (FIG. 26) resulted in an enzyme with both increased activity and specificity. The complete engineering campaign for V379I, showing subsequent rounds of evolution, can be found in FIGS. 27A-27D.

These distinct engineered ACS enzymes possibly resulted from slight tunable differences while screening for enzyme performance in Phase 2. Mutants from the V379I parent were additionally dialyzed after CFPS to remove waste metabolites that accumulate during protein synthesis. Notably, one of these metabolites is acetate. In the presence of acetate, mutations that increased specificity were preferentially identified, while in the absence of acetate, mutations that increased activity were preferentially identified. The tunable reaction environment of CFPS enables engineering strategies that could be inaccessible using other protein production and screening methods.

An embodiment comprising a modified acyl-CoA synthetase (ACS) is provided. A reference acyl-CoA synthetase originating from Erythrobacter sp. NAP1 was used to evolve the disclosed variant acyl-CoA synthetase enzymes, with an amino acid sequence identified in SEQ ID NO: 1:

(SEQ ID NO: 1)
MTGFVERPEQAHTPNCTGVQYAAMYERSLADPDGFWLEQAKRLDWTQQP
RKGGEWSYDPVDIKWFADGSLNLCHNAVDRHLDSRGDTPAIIFEPDDPA
TPSRTLTYRQLHSEVIHMANALKAIGVTKGERVTIYMPNIVEGVTAMLA
CARLGAIHSVVFGGFSPEALAGRIIDCESRFVVTADEGKRGAKSVPLKA
NVDAALEVEGVDVTGVLVVQHTGLAVPMTEGRDHWFHEVKSDADVPCET
MAAEDPLFILYTSGSTGKPKGVLHTTGGYGVWTATTFSYIFDYQPGEVF
WCTADIGWVTGHSYIVYGPLQNGATQVLFEGVPNYPDFGRFWDVVAKHK
VSILYTAPTAIRALMREGDDYVTSRDRSSLRLLGSVGEPINPEAWRWYF
DVVGEGRCPIIDTWWQTETGGCMITTLPGAHDMKPGSAGLPMFGIRPQL
VDNDGAVLDGATEGNLCITHSWPGQARSVYGDHDRFVQTYFSTYSGKYF
TGDGCKRDEDGYYWITGRVDDVINVSGHRMGTAEVESALVLHPQVAEAA
VVGYPHDVKGQGIYCYVTTNAGVEGSDELYQELRAHVRKEIGPIATPDQ
IQFTDGLPKTRSGKIMRRILRKVAENDYGSLGDTSTLADPSLVDRLIEG
RQKT.

In embodiments, a modified ACS was evolved that has a sequence with one or more amino acid substitutions, with reference to SEQ ID NO: 1, at amino acid residue A298, I300, W302, V303, T304, I309, V310, S345, L347, Y348, A350, S378, V379, I383, T405, W406, W407, T409, T411, C414, I513, S516, or A523, or a homolog thereof.

In some embodiments, the modified ACS is selected from the ACS with one or more of the mutations, with reference to SEQ ID NO: 1, having an amino acid substitution at amino acid residue A298, I300, W302, V303, T304, I309, V310, S345, L347, Y348, A350, S378, V379, I383, T405, W406, W407, T409, T411, C414, 1513, S516, or A523, or a homolog thereof.

In some embodiments, the modified ACS evolved has a sequence with one or more amino acid substitutions, with reference to SEQ ID NO: 1, at amino acid residue V303, T304, L347, I383, W407, or A523, or a homologue thereof.

In some embodiments, the modified ACS evolved has a sequence with one or more amino acid substitutions, with reference to SEQ ID NO: 1, at amino acid residue I300, V303, V379, or W407, or a homologue thereof.

### Exemplary Advantages

The disclosed methods and systems possess the non-limiting exemplary advantages over the what is disclosed in the art: improved process speed (>3×), eliminates the need for DNA-sequencing, formyl-CoA synthetases can enable synthetic formate assimilation pathways for the sustainable biomanufacturing of value-added chemicals, improving the rates and specificities of this unnatural reaction is required to enable operation of formate assimilation pathways at an industrially relevant scale, amide synthetase provide an alternative to contemporary synthetic methods that overcomes their inherent limitations. Chemo-, stereo-, and regioselective chemistries can be performed without the need of protecting groups and enzymatic cascades can bypass unfavorable equilibria, amide synthetases are an efficient, economical, environmentally-benign, and sustainable alternative to contemporary synthetic methods. The disclosed chemistry can be performed in buffer, at ambient temperatures and pressures. Enzymes reduce the use of hazardous reagents, produce less waste, display superior atom economy. Enzyme catalysts and cofactors can be recycled for several rounds of biocatalysis, engineered amide synthetases display broad substrates scope, dramatically increased yields for select pharmaceuticals, increased tolerance to temperature, organic solvent, and substrate and product concentrations. The variants of McbA presented here demonstrate McbA's potential as an industrially relevant tool for catalyzing the critically important amide bond.

### Illustrative Embodiments

Several non-limiting embodiments of the present technology are provided below.

1. In a first embodiment, a method for high-throughput generation and expression of site saturated enzyme mutants using cell-free protein synthesis is provided. In some embodiments, the method comprises one or more of the following steps: assembly of linear DNA expression templates containing single sequence-defined mutations without cells via PCR and Gibson Assembly; Expression of enzymes using cell-free protein synthesis; Evaluation of enzyme fitness using liquid-chromatography or fluorescent assays.; Identifying the mutant with the highest fitness and repeating the workflow; Applying machine learning algorithms to explore sequences not found in the initial directed evolution campaign.

2. The method of embodiment 1 where DNA sequences are identified via computational methods for predicting protein structure.

3. The method of embodiment 1 where DNA sequences are identified via bioinformatic tools for prediction evolutionary conservation.

4. The method of embodiment 1 where the workflow is automated.

5. An embodiment comprising a modified acyl-CoA synthetase is provided. In some embodiments, the modified acyl-CoA synthetase is selected from the following group or a homolog thereof:
(a) a modified acyl-CoA synthetase (ACS) originating from Erythrobacter sp. NAP1 (SEMTGFVERPEQAHTPNCTGVQYAAMYERSLADPDGFWLEQAKRLDWTQQPRKG GEWSYDPVDIKWFADGSLNLCHNAVDRHLDSRGDTPAIIFEPDDPATPSRTLTYRQLH SEVIHMANALKAIGVTKGERVTIYMPNIVEGVTAMLACARLGAIHSVVFGGFSPEAL AGRIIDCESRFVVTADEGKRGAKSVPLKANVDAALEVEGVDVTGVLVVQHTGLAVP MTEGRDHWFHEVKSDADVPCETMAAEDPLFILYTSGSTGKPKGVLHTTGGYGVWTA TTFSYIFDYQPGEVFWCTADIGWVTGHSYIVYGPLQNGATQVLFEGVPNYPDFGRFW DVVAKHKVSILYTAPTAIRALMREGDDYVTSRDRSSLRLLGSVGEPINPEAWRWYFD VVGEGRCPIIDTWWQTETGGCMITTLPGAHDMKPGSAGLPMFGIRPQLVDNDGAVL DGATEGNLCITHSWPGQARSVYGDHDRFVQTYFSTYSGKYFTGDGCKRDEDGYYWI TGRVDDVINVSGHRMGTAEVESALVLHPQVAEAAVVGYPHDVKGQGIYCYVTTNA GVEGSDELYQELRAHVRKEIGPIATPDQIQFTDGLPKTRSGKIMRRILRKVAENDYGS LGDTSTLADPSLVDRLIEGRQKT (SEQ ID NO: 1)) comprising one or more substitutions at amino acid positions A298, I300, W302, V303, T304, I309, V310, S345, L347, Y348, A350, S378, V379, I383, T405, W406, W407, T409, T411, C414, I513, S516, or A523;
(b) a modified acyl-CoA synthetase of embodiment (a) comprising one or more substitutions in combination at amino acid positions V303, T304, L347, I383, W407, or A523 for the production of formyl-CoA;
(c) a modified acyl-CoA synthetase of embodiment (a) comprising one or more substitutions in combination at amino acid positions I300, V303, V379, or W407 for the production of formyl-CoA; or
(d) a modified acyl-CoA synthetase of any of the previous embodiments, when expressed in an Escherichia coli strain with a genomic modification removing or inactivating the enzyme L-lysine acetyltransferase (corresponding to the gene patZ).

6. A modified amide synthetase selected from the following group or a homolog thereof:
(a) a modified amide synthetases “McbA” Marinasctinospora thermotolerans (MEKKIWSHPQFEKGGSGENLYFQGGYARRVMDGIGEVAVTGAGGSVTGARLRHQV RLLAHALTEAGIPPGRGVACLHANTWRAIALRLAVQAIGCHYVGLRPTAAVTEQARA IAAADSAAL VFEPSVEARAADLLERVSVPVVLSLGPTSRGRDILAASVPEGTPLRYRE HPEGIAVVAFTSGTTGTPKGVAHSSTAMSACVDAAVSMYGRGPWRFLIPIPLSDLGGE LAQCTLATGGTVVLLEEFQPDAVLEAIERERATHVFLAPNWLYQLAEHPALPRSDLSS LRRVVYGGAPAVPSRVAAARERMGAVLMQNYGTQEAAFIAALTPDDHARRELLTA VGRPLPHVEVEIRDDSGGTLPRGAVGEVWVRSPMTMSGYWRDPERTAQVLSGGWL RTGDVGTFDEDGHLHLTDRLQDIIIVEAYNVYSRR VEHVLTEHPDVRAAAVVGVPDP DSGEAVCAAVVVADGADPDPEHLRAL VRDHLGDLHVPRR VEFVRSIPVTPAGKPDK VKVRTWFTD (SEQ ID NO: 2)) comprising one or more substitutions at amino acid positions Y97, R101, P102, T103, V177, V178, A179, T181, V191, H193, A197, M198, C201, A205, Y209, I220, P221, D224, L225, E228, L229, C232, E244, E245, F246, F264, L265, A266, W269, V292, G293, G294, A295, P296, A297, Q315, N316, Y317, G318, T319, Q320, E321, A323, F324, A341, M375, T376, D400, L412, D414, R415, I421, E423, A424, Y425, N426, R430, L487, P503, A504, G505, K506, P507, and D508
(b) a modified amide synthetases “McbA” Marinasctinospora thermotolerans (above) comprising one or more substitutions at amino acid positions P102, T103, V177, I220, C232, A266, A323, or R430 for the production of moclobemide
(c) modified amide synthetases “McbA” Marinasctinospora thermotolerans (above) comprising one or more substitutions at amino acid positions P102, T103, V177, C201, A205, C232, A424, R430 for the production of cinchocaine
(d) a modified amide synthetases “McbA” Marinasctinospora thermotolerans (above) comprising one or more substitutions at amino acid positions P102, T103, V177, C201, I2220, L225, E244, A266, A295, G318, T319, Q320, A323, I421, E423, A424, Y425, R430, or D508 for the production of metoclopramide, procainamide, or declopramide;
(e) a modified amide synthetases “McbA” Marinasctinospora thermotolerans (above) comprising one or more substitutions at amino acid positions Y97, P102, T103, V177, C201, I220, P221, L225, E228, C232, E244, A266, N316, T319, A323, L412, E423, A424, Y425, or R430 for the production of Itopride, or trimethobenzamide;
(f) a modified amide synthetases “McbA” Marinasctinospora thermotolerans (above) comprising one or more substitutions at amino acid positions P102, T103, V177, C201, A205, Y209, I220, P221, L225, C232, E244, A266, A295, Y317, G318, T319, Q320, A323, T376, E423, A424, Y425, or R430 for the production of Sulpiride or troxipide.

### EXAMPLES

The following Examples are illustrative and should not be interpreted to limit the scope of the claimed subject matter.

The strategy was to leverage the flexibility and speed provided by cell-free DNA-assembly³⁷ and protein synthesis (CFPS) to build and test site-saturated, sequence-defined libraries (FIG. 1). After amino acid residue selection based on structural insights, evolutionary trends, and design tools (e.g., ROSETTA³⁸, EVmutation²², PROSS³⁹), the disclosed workflow has five steps for high-throughput, cell-free DNA template assembly and expression: (i) a DNA primer containing a mismatch introduces a desired mutation through PCR, (ii) the parent plasmid is digested, (iii) an intramolecular Gibson assembly forms a mutated plasmid, (iv) a second PCR amplifies linear DNA expression templates (LETs), and (v) the mutated protein is expressed through CFPS. In this way, hundreds to thousands of sequence-defined protein mutants can be built and their function can be tested in individual reactions within 24 hours.

The disclosed workflow was validated using the well-characterized, monomeric ultra-stable green fluorescent protein⁴⁰ (muGFP) by targeting four residues that are known to be important for stability and that compose the chromophore^{41,42} The workflow was applied to engineer selected amide synthetase McbA, focusing on the synthesis of the monoamine oxidase A inhibitor moclobemide (FIGS. 2A-2C), and an acetyl-CoA synthetase, focusing on formyl-CoA synthesis.

### Example 1—A Cell-Free Protein Evolution Platform to Rapidly Screen Sequence-Defined Libraries

### Phase 1—Cell-Free Library Generation and Protein Synthesis

### Step 1: Choose Desired Mutations via Hot Spot Screening

### Step 2: Introduce Desired Mutations via PCR

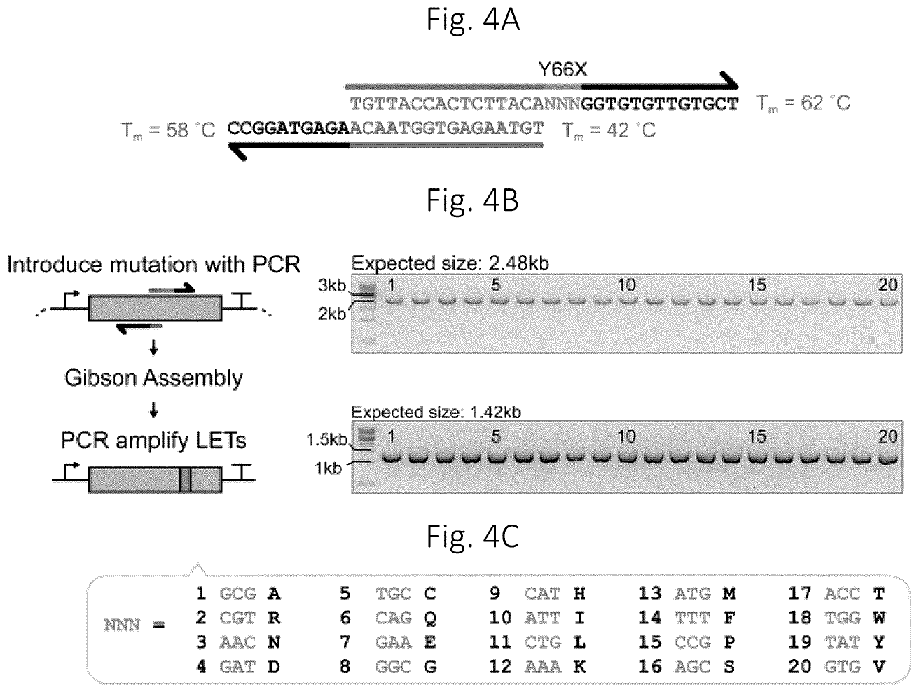

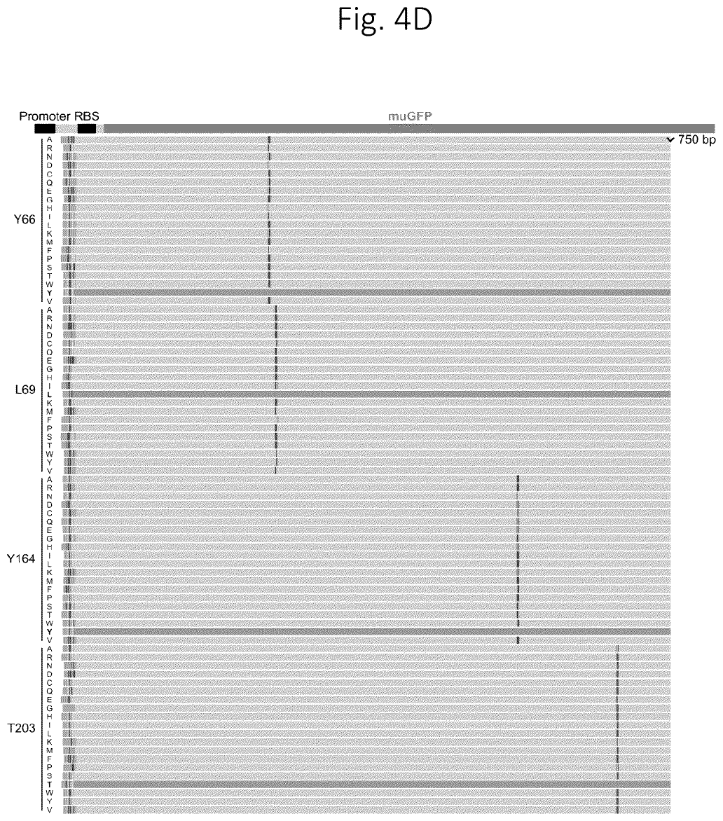

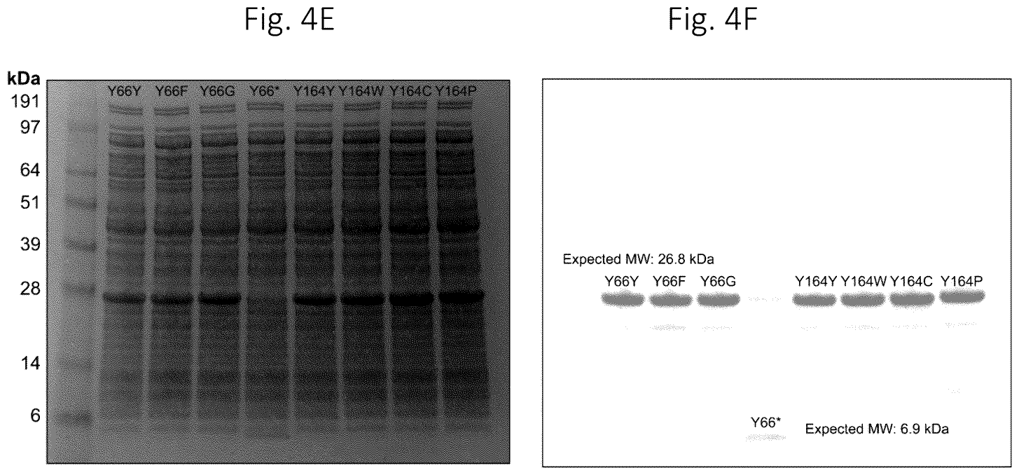

Primers were designed using Benchling with melting temperature calculated by the default SantaLucia 1998 algorithm. The general heuristics for primer design were a reverse primer of 58° C., a forward primer of 62° C., and a homologous overlap of approximately 45° C. All primers were ordered from Integrated DNA Technologies (IDT); forward primers were synthesized in 384-well plates normalized to 2-μM for ease of setting up reactions. (FIGS. 4A-4D).

The following codons were used in the forward primers in our cell-free DNA assembly workflow to mutate a desired residue into the corresponding amino acid. While the addition of excess tRNA in CFPS reactions mitigates the negative effects of unoptimized codons, we used the most prevalent codon found in E. coli for the compatibility of in vivo expression and to prevent the need for re-optimizing the entire sequence.

### Codon Table for Designing Site Saturation Mutagenesis Primers

| Amino Acid | Codon |
|---|---|
| A | GCG |
| R | CGT |
| N | AAC |
| D | GAT |
| C | TGC |
| Q | CAG |
| E | GAA |
| G | GGC |
| H | CAT |
| I | ATT |
| L | CTG |
| K | AAA |
| M | ATG |
| F | TTT |
| P | CCG |
| S | AGC |
| T | ACC |
| W | TGG |
| Y | TAT |
| V | GTG |

The base forward (fwd) and reverse (rvs) primers for site saturation mutagenesis of wt-McbA using our cell-free DNA assembly workflow. While there is only a single reverse primer for saturating a single residue, the forward primer carries the desired mutation (so there are subsequently 20 forward primers per residue). This flexible position was labeled as ‘NNN’, indicating that all 20 codons found in codon table above are inserted here.

### Primers for Site Saturation Mutagenesis of wt-McbA

| | | SEQ ID | |
|---|---|---|---|
| Residue | Direction | NO | Sequence |
| Y97 | fwd | 3 | CGATTGGTTGCCACNNNGTTGGTCTGCG |
| | rvs | 4 | GTGGCAACCAATCGCCTGAACA |
| R101 | fwd | 5 | CCACTATGTTGGTCTGNNNCCTACCGC |
| | rvs | 6 | CAGACCAACATAGTGGCAACCAATCG |
| P102 | fwd | 7 | TGTTGGTCTGCGTNNNACCGCTG |
| | rvs | 8 | ACGCAGACCAACATAGTGGCAAC |
| T103 | fwd | 9 | GGTCTGCGTCCTNNNGCTGCTG |
| | rvs | 10 | AGGACGCAGACCAACATAGTGGC |
| V177 | fwd | 11 | CAGAAGGTATCGCANNNGTAGCCTTTACTAGCG |
| | rvs | 12 | TGCGATACCTTCTGGGTGTTCACG |
| V178 | fwd | 13 | GAAGGTATCGCAGTTNNNGCCTTTACTAGCGG |
| | rvs | 14 | AACTGCGATACCTTCTGGGTGTTCA |
| A179 | fwd | 15 | GGTATCGCAGTTGTANNNTTTACTAGCGGC |
| | rvs | 16 | TACAACTGCGATACCTTCTGGGTGTTC |
| T181 | fwd | 17 | GCAGTTGTAGCCTTTNNNAGCGGCACCA |
| | rvs | 18 | AAAGGCTACAACTGCGATACCTTCTGG |
| V191 | fwd | 19 | CACCCCTAAAGNNNCGGCCCACTC |
| | rvs | 20 | GCCTTTAGGGGTGCCAGTGGT |
| H193 | fwd | 21 | TAAAGGCGTTGCCNNNTCCTCTACCG |
| | rvs | 22 | GGCAACGCCTTTAGGGGTGC |
| A197 | fwd | 23 | CCCACTCCTCTACCNNNATGAGCGC |
| | rvs | 24 | GGTAGAGGAGTGGGCAACGC |
| M198 | fwd | 25 | CTCCTCTACCGCTNNNAGCGCTTGTGTG |
| | rvs | 26 | AGCGGTAGAGGAGTGGGCAAC |
| C201 | fwd | 27 | GCTATGAGCGCTNNNGTGGATGCTGC |
| | rvs | 28 | AGCGCTCATAGCGGTAGAGGAG |
| A205 | fwd | 29 | CTTGTGTGGATGCTNNNGTTTCCATGT |
| | rvs | 30 | AGCATCCACACAAGCGCTCATAG |
| Y209 | fwd | 31 | TGCGGTTTCCATGNNNGGTCGCG |
| | rvs | 32 | CATGGAAACCGCAGCATCCACA |
| I220 | fwd | 33 | GTTTCCTGATCCCGNNNCCTCTGTCTGAC |
| | rvs | 34 | CGGGATCAGGAAACGCCAAGG |
| P221 | fwd | 35 | TCCTGATCCCGATCNNNCTGTCTGACC |
| | rvs | 36 | GATCGGGATCAGGAAACGCCAAG |
| D224 | fwd | 37 | CGATCCCTCTGTCTNNNCTGGGTGG |
| | rvs | 38 | AGACAGAGGGATCGGGATCAGGA |
| L225 | fwd | 39 | TCCCTCTGTCTGACNNNGGTGGC |
| | rvs | 40 | GTCAGACAGAGGGATCGGGATCAG |
| E228 | fwd | 41 | GACCTGGGTGNNNCGCTGGCAC |
| | rvs | 42 | GCCACCCAGGTCAGACAGAGG |
| L229 | fwd | 43 | CTGGGTGGCGAANNNGCACAGTG |
| | rvs | 44 | TTCGCCACCCAGGTCAGACAG |
| C232 | fwd | 45 | CGAACTGGCACAGNNNACCCTGGC |
| | rvs | 46 | CTGTGCCAGTTCGCCACCC |
| E244 | fwd | 47 | CGTTGTGCTGCTGNNNGAGTTCCAACC |
| | rvs | 48 | CAGCAGCACAACGGTACCGC |
| E245 | fwd | 49 | TGTGCTGCTGGAANNNTTCCAACCG |
| | rvs | 50 | TTCCAGCAGCACAACGGTACC |
| F246 | fwd | 51 | GCTGCTGGAAGAGNNNCAACCGGAC |
| | rvs | 52 | CTCTTCCAGCAGCACAACGGTAC |
| F264 | fwd | 53 | GCCACTCACGTGNNNCTGGCG |
| | rvs | 54 | CACGTGAGTGGCACGTTCACG |
| L265 | fwd | 55 | CCACTCACGTGTTONNNGCGC |
| | rvs | 56 | GAACACGTGAGTGGCACGTTCA |
| A266 | fwd | 57 | CTCACGTGTTCCTGNNNCCGAA |
| | rvs | 58 | CAGGAACACGTGAGTGGCACG |
| W269 | fwd | 59 | TGGCGCCGAACNNNCTGTACC |
| | rvs | 60 | GTTCGGCGCCAGGAACACG |
| V292 | fwd | 61 | CGTCGCGTTGTTNNNGGCGGTG |
| | rvs | 62 | AACAACGCGACGCAGAGAAGAC |
| G293 | fwd | 63 | GTCGCGTTGTTTACNNNGGTGC |
| | rvs | 64 | GTAAACAACGCGACGCAGAGAAGA |
| G294 | fwd | 65 | CGTTGTTTACGNNNCGGCACCG |
| | rvs | 66 | GCCGTAAACAACGCGACGC |
| A295 | fwd | 67 | TGTTTACGNNNGTGCACCGG |
| | rvs | 68 | ACCGCCGTAAACAACGCGAC |
| P296 | fwd | 69 | CGGCGGTGCANNNGCAG |
| | rvs | 70 | TGCACCGCCGTAAACAACGC |
| A297 | fwd | 71 | CGGTGCACCGNNNGTACCATCTC |
| | rvs | 72 | CGGTGCACCGCCGTAAACA |
| Q315 | fwd | 73 | TGCTGTGCTGATGNNNAACTACGGC |
| | rvs | 74 | CATCAGCACAGCACCCATACGTTC |
| N316 | fwd | 75 | TGTGCTGATGCAGNNNTACGGCACC |
| | rvs | 76 | CTGCATCAGCACAGCACCCATAC |
| Y317 | fwd | 77 | TGCTGATGCAGAACNNNGGCACCC |
| | rvs | 78 | GTTCTGCATCAGCACAGCACCC |
| G318 | fwd | 79 | CTGATGCAGAACTACNNNACCCAGGAA |
| | rvs | 80 | GTAGTTCTGCATCAGCACAGCACC |
| T319 | fwd | 81 | GCAGAACTACGNNNCGCAGGAAGC |
| | rvs | 82 | GCCGTAGTTCTGCATCAGCACAG |
| Q320 | fwd | 83 | GAACTACGGCACCNNNGAAGCAGC |
| | rvs | 84 | GGTGCCGTAGTTCTGCATCAGC |
| E321 | fwd | 85 | TACGGCACCCAGNNNGCAGCTTTCA |
| | rvs | 86 | CTGGGTGCCGTAGTTCTGCATC |
| A323 | fwd | 87 | CACCCAGGAAGCANNNTTCATCGCAG |
| | rvs | 88 | TGCTTCCTGGGTGCCGTAGT |
| F324 | fwd | 89 | CCAGGAAGCAGCTNNNATCGCAGCA |
| | rvs | 90 | AGCTGCTTCCTGGGTGCC |
| A341 | fwd | 91 | GTGAACTGCTGACCNNNGTAGGTCGT |
| | rvs | 92 | GGTCAGCAGTTCACGACGTGC |
| M375 | fwd | 93 | GTACGTTCCCCGNNNACTATGTCTGGTTACTGG |
| | rvs | 94 | CGGGGAACGTACCCAGACTTCA |
| T376 | fwd | 95 | ACGTTCCCCGATGNNNATGTCTGGTTACTGG |
| | rvs | 96 | CATCGGGGAACGTACCCAGACT |
| D400 | fwd | 97 | GCTGCGTACTGGTNNNGTTGGTACCTTC |
| | rvs | 98 | ACCAGTACGCAGCCAACCAC |
| L412 | fwd | 99 | TGGTCACCTGCATNNNACCGATCGTC |
| | rvs | 100 | ATGCAGGTGACCATCCTCATCGAAG |
| D414 | fwd | 101 | CCTGCATCTGACCNNNCGTCTGCAG |
| | rvs | 102 | GGTCAGATGCAGGTGACCATCCT |
| R415 | fwd | 103 | TGCATCTGACCGATNNNCTGCAGGAC |
| | rvs | 104 | ATCGGTCAGATGCAGGTGACCATC |
| I421 | fwd | 105 | TGCAGGACATCATCNNNGTTGAAGCATATAACGTC |
| | rvs | 106 | GATGATGTCCTGCAGACGATCGGT |
| E423 | fwd | 107 | GGACATCATCATCGTTNNNGCATATAACGTCTATTCCCG |
| | rvs | 108 | AACGATGATGATGTCCTGCAGACGA |
| A424 | fwd | 109 | CATCATCATCGTTGAANNNTATAACGTCTATTCCCGTCG |
| | rvs | 110 | TTCAACGATGATGATGTCCTGCAGAC |
| Y425 | fwd | 111 | ATCATCGTTGAAGCANNNAACGTCTATTCCCGTCGTG |
| | rvs | 112 | TGCTTCAACGATGATGATGTCCTGC |
| N426 | fwd | 113 | CATCGTTGAAGCATATNNNGTCTATTCCCGTCGTG |
| | rvs | 114 | ATATGCTTCAACGATGATGATGTCCTGC |
| R430 | fwd | 115 | GCATATAACGTCTATTCCNNNCGTGTGGAACATG |
| | rvs | 116 | GGAATAGACGTTATATGCTTCAACGATGATGATG |
| L487 | fwd | 117 | ATCACCTGGGTGATNNNCACGTTCCTC |
| | rvs | 118 | ATCACCCAGGTGATCACGAACCAG |
| P503 | fwd | 119 | CCATCCCGGTAACTNNNGCCGG |
| | rvs | 120 | AGTTACCGGGATGGAGCGAACG |
| A504 | fwd | 121 | CCCGGTAACTCCTNNNGGCAAAC |
| | rvs | 122 | AGGAGTTACCGGGATGGAGCG |
| G505 | fwd | 123 | GGTAACTCCTGCCNNNAAACCAGATAAAGT |
| | rvs | 124 | GGCAGGAGTTACCGGGATGGAG |
| K506 | fwd | 125 | CTCCTGCCGNNNCGCCAGATAAAGTGAAAGT |
| | rvs | 126 | GCCGGCAGGAGTTACCGG |
| P507 | fwd | 127 | CCTGCCGGCAAANNNGATAAAGTGAAAGTG |
| | rvs | 128 | TTTGCCGGCAGGAGTTACCG |
| D508 | fwd | 129 | GCCGGCAAACCANNNAAAGTGAAAGTGCG |
| | rvs | 130 | TGGTTTGCCGGCAGGAGTTAC |

The forward (fwd) and reverse (rvs) primers for site saturation mutagenesis of muGFP using the cell-free DNA assembly workflow. the primers used in the optimization of the homologous overlap between the two primers found in FIG. 2B are also included. The temperature next to the primer for Y66 corresponds to the melting temperature of the overlap.

### Primers for Site Saturation Mutagenesis of muGFP

| | | SEQ ID | |
|---|---|---|---|
| Residue | Direction | NO | Sequence |
| Y66 | fwd-27° C. | 131 | CCACTCTTACATATGGTGTGTTGTGCTTTAGC |
| | fwd-33° C. | 132 | TACCACTCTTACATATGGTGTGTTGTGCTTTA |
| | fwd-42° C. | 133 | TGTTACCACTCTTACATATGGTGTGTTGTGCT |
| | fwd-48° C. | 134 | CTCTTGTTACCACTCTTACATATGGTGTGTTGTG |
| | fwd-53° C. | 135 | CCTACTCTTGTTACCACTCTTACATATGGTGTGTTG |
| | rvs | 136 | TGTAAGAGTGGTAACAAGAGTAGGCC |
| L69 | fwd | 137 | CTCTTACATATGGTGTGTTGTGCTTTAGCCG |
| | rvs | 138 | CACACCATATGTAAGAGTGGTAACAAGAG |
| Y164 | fwd | 139 | AATGGGATCAAAGCATACTTCAAAATCCGC |
| | rvs | 140 | TGCTTTGATCCCATTTTTTTGCTTATCAG |
| T203 | fwd | 141 | CAATCACTACCTTAGCACACAGTCGGTATT |
| | rvs | 142 | GCTAAGGTAGTGATTGTCTGGCAAC |

The forward primers for amplifying LETs are universally used to amplify LETs off pJL1 containing any gene of interest. They add approximately 300 basepairs both upstream and downstream of the coding region to help protect against exonucleases present in the cell extract.

### Forward Primers for Amplifying LETs Using pJL1 Plasmids as a Template

| Direction | SEQ ID NO | Sequence |
|---|---|---|
| LET_fwd | 143 | CTGAGATACCTACAGCGTGAGC |
| LET_rvs | 144 | CGTCACTCATGGTGATTTCTCACTTG |

All cloning steps were set up using an Integra VIAFLO liquid handling robot in 384-well PCR plates (Bio-Rad). The first PCR was performed in a 10-μL reaction with 1-ng of plasmid template added, then 1-μL of DpnI was added and incubated at 37° C. for two hours.

### Step 3: Use Gibson Assembly to Construct Mutated Plasmids

The products for Step 2 was diluted 1:4 by the addition of 29-μL of nuclease-free (NF) water, (4) 1-μL of diluted DNA and added to a 3-μL Gibson assembly reaction and incubated for 50° C. for one hour. Then the assembly reaction was diluted 1:10 by the addition of 36-μL of NF water. All machine learning predicted McbA variants were ordered as eblocks from IDT containing pJL1 (Addgene, 69496) 5′ and 3′ Gibson assembly overhangs. DNA was resuspended at a concentration of 25 ng/μL. A linearized pJL1 plasmid backbone was ordered as a gblock from IDT, PCR amplified, purified using a DNA Clean and Concentrate Kit (Zymo Research), and diluted to a concentration of 50 ng/μL. Gibson assembly was used to assemble the DNA encoding McbA variants with the pJL1 backbone. 10 ng of purified, linearized pJL1 backbone and 10 ng of eblock insert were combined in a 3-μL Gibson assembly reaction and incubated at 50° C. for 30 minutes³⁷. The unpurified assembly reactions were diluted in 60-μL of NF water and 1-μL of the diluted reaction was used as the template for a 50-μL PCR reaction (using Q5 Hot Start DNA polymerase) to generate LETs for CFPS.

### Step 4: Amplify Linear Expression Templates (LETs) via PCR

1-μL of the diluted assembly reaction from Step 3 was added to a 9-μL PCR reaction. All PCR reactions used Q5 Hot Start DNA Polymerase (NEB). The thermocycler parameters were consistent throughout this study, with extension time being the only variable changing to compensate for different amplicon lengths. The first step uses touchdown PCR, in which the initial annealing temperature decreases by 1° C. each cycle until a final set temperature was reached.

The following thermocycler parameters were consistent throughout the study, with extension time being the only variable changing to compensate for different amplicon lengths. The first step uses touchdown PCR, in which the initial annealing temperature decreases by 1° C. each cycle until a final set temperature was reached.

### Thermocycler Parameters for Cell-Free DNA Assembly

PCR 1 parameters:

| Step | Temp (° C.) | Time (min:sec) |
|---|---|---|
| Initial Denaturation | 98 | 3:00 |
| 6x | 98 | 0:30 |
| | 70 (−1° C./cycle) | 0:30 |
| | 72 | 20 s/kbp |
| 20x | 98 | 0:30 |
| | 64 | 0:30 |
| | 72 | 20 s/kbp |
| Final Extension | 72 | 10:00 |
| Hold | 12 | ∞ |

PCR 2 parameters:

| Step | Temp (° C.) | Time (min:sec) |
|---|---|---|
| Initial Denaturation | 98 | 3:00 |
| 30x | 98 | 0:30 |
| | 68 | 0:30 |
| | 72 | 20 s/kbp |
| Final Extension | 72 | 10:00 |
| Hold | 12 | ∞ |

To accumulate mutations for ISM, 3-μL of the “winner” from the diluted Gibson assembly plate was transformed into 20-μL of chemically competent E. coli (NEB 5-alpha cells). Cells were plated onto LB plates containing 50 μg/mL kanamycin (LB-Kan). A single colony was used to inoculate a 50 mL overnight culture of LB-Kan, grown at 37° C. with 250 RPM shaking. The plasmid was purified using ZymoPURE II Midiprep kits and sequence confirmed.

### Step 5: Use Cell-Free Protein Synthesis (CFPS) to Express Mutated Genes

Crude cell extracts were prepared as previously described using E. coli BL21 Star (DE3) cells (Invitrogen)⁶². CFPS reactions were performed based on the Cytomim system^{63,64} and carried out in 384-well PCR plates (Bio-Rad) as 10-μL reactions with 1-μL of LET serving as the DNA template.

Phase 2—Quantify performance of mutants: this pipeline can be used to find mutations that improve a desired function of a given protein. The method to quantify the performance of a mutation depends on the desired function. Here the inventors list several options to assess the performance of a protein, specifically to quantify amide synthetase reactions and products. It should be readily apparent to one skilled in the art that different methods of assessing the performance of mutants can be used based on the desired function.

### Analytics

Amide products (along with acid substrates and some adenylated acid intermediates) were analyzed using an Agilent G6125B Single Quadrupole LC/MSD system equipped with an electrospray ionization source set to positive ionization mode. The quenched samples were centrifuged for 10 min at 4,500×g to remove precipitated proteins. A separate 384-well plate for sample injection into the HPLC-MS was prepared by diluting 5 μL of the quenched samples with 25 μL of methanol using the Integra VIAFLO. Trace amounts of compounds were detected using MS, while many compounds were present in high enough concentration to quantify by diode array detector (DAD) at 254 nm. Compounds were separated on a Luna C18 Column (Phenomenex 00D-4251-B0) using mobile phases (A) H₂O with 0.1% formic acid and (B) Acetonitrile. The general method for chromatographic separation was carried out using the following gradients at a constant flow rate of 0.5 mL/min: 0 min 5% B; 1 min 5% B; 4 min 95% B; 4.5 min 95% B; 5 min 5% B. For the MS, capillary voltage was set at 3 kV, and nitrogen gas was used for nebulizing (35 psig) and drying (12 l/min, 350° C.). The MS was calibrated using Tuning Mix (Agilent G2421-60001) before measurements were taken. MS data were acquired with a scan range of 50-600 m/z with various SIM m/z's according to which compound the inventors were screening for. LC-MS data were collected and analyzed using Agilent OpenLab CDS ChemStation software. The product yield was calculated by dividing the DAD peak area for the amide product by the sums of the peak areas of both the amide and the acid substrate.

Protein melting temperature was determined using a Jasco J-810 circular dichroism spectrophotometer with a 10 mm path length cuvette monitored at 222 nm. McbA samples were first buffer exchanged into a 1× phosphate buffered saline solution, pH 7.4, and diluted to 0.2-0.4 mg/mL.

### Enzyme Kinetics

McbA apparent kinetics for the amine pair of moclobemide (4-(2-aminoethyl)morpholine) were determined by enzymatically coupling amide bond formation (and the concomitant release of AMP from the acyl-AMP intermediate by its substitution with the amine) with the oxidation of NADH, as shown in FIGS. 7A-7B. Reactions contained 100 mM MOPS-KOH pH 7.8, 5 mM MgCl₂, 2.5 mM phosphoenolpyruvate, 5 mM ATP, 0.3 mM NADH, 50 mM 4-chlorobenzoic acid, 15 U/mL pyruvate kinase and lactate dehydrogenase enzyme mix (Sigma-Aldrich P0294), 25 U/mL myokinase (Sigma-Aldrich 475941), and various concentrations (50-200 μg/mL) of the studied McbA variant. As the acid here (4-chlorobenzoic acid) has poor solubility in water and was dissolved in DMSO, the final reactions contained 10% v/v DMSO (equivalent to our amidation screens). 180-μL reactions were first equilibrated at 30° C. for 3 minutes and then initiated by adding 20-μL of amine. The initial velocity was determined for different concentrations of amine (0.1 mM-50 mM) by measuring NADH absorbance at 340 nM on a Cary 60 UV-Vis (Agilent). Data were collected and analyzed using the Cary WinUV Kinetics Application software (Agilent). Michaelis-Menten graphs were plotted in GraphPad Prism and fit using the default Michaelis-Menten non-linear regression analysis tool.

Kinetics for the acid pair of moclobemide (4-chlorobenzoic acid) were measured similarly as described above, except the amine was held constant at 50 mM and the reaction was initiated by addition of various amounts of the acid. The final DMSO concentration was still held constant at 10% v/v. The inventors observed non-Michaelis-Menten behavior when attempting to determine the kinetics for the acid, in what appeared to be substrate inhibition by the acid (data not shown). The inventors also attempted to measure the acid adenylation step directly by enzymatically coupling acyl-AMP formation (and the concomitant release of PPᵢ) with the oxidation of NADH to further probe the reaction mechanism. The Piper™ pyrophosphate assay kit (Fisher Scientific P22062) was used, but the addition of small concentrations of DMSO resulted in the precipitation of enzymes found in the kit.

Phase 3—Iterative site saturation mutagenesis (ISM): after Phase 1 and Phase 2 in the first iteration, the best performing mutant was selected and tested to determine whether further mutations would be beneficial. The mutant was selected, then another key residue was chosen and plasmids containing each mutation were prepared following the above protocol except 5 mL LB-Kan overnights were used to purify plasmids using ZymoPURE II Miniprep kits. These 20 plasmids were used as templates for the next round of site saturation mutagenesis to accumulate all 400 double mutants. This process can be iterated as many times as necessary to select a high-performing mutant.

Phase 4—Using Machine Learning to Inform Iterative Site Selection: selective methods predict what residues will have desired effects when mutated. This can be used in Phase 1 Step 1, Hot Spot Screening, or, this phase can be used based on of the best performing products of Phase 2 and Phase 3. Using machine learning can construct a landscape of potentially useful locations for mutations that can be explored.

### Amino Acid Encodings

Five different amino acid encoding strategies were studied here following the work of Wittman et al. and Vornholt et al.^{16,67}: one-hot, Georgiev, VHSE, z-scales, and physical descriptors. Beyond one-hot encodings (that contain no information about the nature of the amino acid at each position), encodings that attempt to encapsulate physiochemical properties of amino acids are included. To make informative numerical representations of amino acid properties, these strategies perform principal component analysis (PCA) of different manually curated sets of either experimentally measured or computationally predicted/estimated properties. Georgiev⁴⁵ features (19-parameters) are principal components of the over 500 amino acid indices taken from the AAindex database. VHSE⁴⁶ features (8-parameters) are principal components of 50 variables, focused on hydrophobic, steric, and electronic properties. Z-scales⁴⁷ (5-parameters) features are principal components of 26 variables, focused on lipophilicity, size, and polarity. Physical descriptors^{48,68} (3-parameters) features are derived from a rational ad hoc modification of principal components of hydrophobic and steric properties of peptides. For all strategies, the inventors first generated encodings for the entire combinatorial library tested (stored in a tensor of “420 unique variants”×“4 amino acids”×“n-parameters”, where n-parameters is equal to the number of amino acids for one-hot). The last two dimensions of the tensor were then flattened to generate a matrix. Specifically for the physiochemical encodings (excluding one-hot), each column of the matrix was standardized (mean-centered and unit-scaled).

### Zero-Shot Predictions

Evolutionary: The EVmutations²² probability density model was trained using the EVcouplings webserver (evcouplings.org/) with default parameters, with the input sequence for McbA taken from UniProt (R4R1U5). The model the inventors selected had a bitscore inclusion threshold of 0.7. The model and code for replicating zero-shot predictions are provided in our GitHub repository. The mutation effects prediction code provided in the EVcouplings GitHub repository (github.com/debbiemarkslab/EVcouplings/blob/develop/notebooks/model_parameters_mutation_effects.ipynb) was used as a template. Features for the augmented models were derived from the sequence statistical energy relative to wild type.

Universal: Predictions using the ESM-1b¹³ pre-trained transformer language model were made using the code provided from the excellent work of Wittman et. al on machine learning-guided directed evolution (github.com/fhalab/MLDE) with the ESM-1b model provided in the ESM GitHub repository (github.com/facebookresearch/esm). Briefly, a mask-filling protocol was used to predict the probability of different mutants by presenting the model with the entire sequence and “masking” a position of interest. A naïve mask-filing approach was used, which considers each variable position as independent from each other. This mask-filing approach is less computationally expensive and provided slightly superior predictions than a conditional approach (which does not assume independence of variable positions) in this previous work. A complete description of the code can be found in the original publication and the associated GitHub repository. Features for the augmented models were derived from the sequence log-probability relative to wild type.

Structural: Structural-based predictions were made using the MAESTRO⁴⁹ command line tool for Windows (v1.2.35). The Protein Data Bank (PDB) structure for McbA (6SQ8) was used as the input and calculated changes in stability (unfolding free energy) with the ‘evalmut’ command. Features for the augmented models were derived using the ‘energy’ output.

### Machine Learning Guided Directed Evolution

Ridge regression models were augmented following the code accompanying the work of Hsu et al.²⁶ (github.com/chloechsu/combining-evolutionary-and-assay-labelled-data). McbA variant sequence featurization was performed by concatenating zero-shot predictions with site-specific amino acid encodings. Zero-shot predictions were first standardized and regularized by a common regularization strength (10-8). The L2 regularization strength for ridge regression (α) was determined during hyperparameter tuning using cross-validation. For our complete code and to replicate our results and predictions made in this work, please see our accompanying GitHub repository.

Model evaluation and selection were first performed retrospectively by using the assay-labeled datasets from our moclobemide and metoclopramide engineering campaigns. Augmented models (using combinations of the above zero-shot predictors and amino acid encodings) were trained on the single site saturation libraries for four residues (n≈80) and tested on the withheld higher-order mutants from the additional rounds of saturation mutagenesis (n≈200). Hyperparameter turning of α was performed using repeated 5-fold cross-validation (with 20 repeats) by randomly sampling 80% of the training data and testing on the withheld 20%; model performance was evaluated using mean squared error (MSE). With the optimized hyperparameter, all trained models were used to make predictions on the withheld test set. Spearman correlation coefficient and NDCG were used to select the best zero-shot predictor and encoding strategy, with a preference given to NDCG. After identifying the best model (which in our case was augmenting the EVmutation probability density model with Georgiev encodings), the inventors made predictions on the entire combinatorial dataset (n=160,000). The top 25 predictions for moclobemide and metoclopramide were then experimentally tested (FIG. 4). Model training and predictions for the remaining seven amide products were performed similarly as above.

### Data Collection and Analysis

All statistical information provided in this manuscript is derived from n=3 independent experiments unless otherwise noted in the text or figure legends. Error bars represent 1 s.d. of the mean derived from these experiments. Data analysis and figure generation were conducted using Excel Version 2304, ChimeraX Version 1.5⁶⁹, GraphPad Prism Version 9.5.0, and Python 3.9 using custom scripts available on GitHub. muGFP fluorescence was measured on a BioTek Synergy H1 Microplate Reader and analyzed using Gen5 Version 2.09.2. Autoradiograms were performed as previously described and scanned using the Typhoon FLA 7000 Imager v1.2⁷⁰.

### Example 2—Using Cell-Free Evolution to Improve GFP Signal

The workflow was validated using the well-characterized, monomeric ultra-stable green fluorescent protein⁴⁰ (muGFP) by targeting four residues that are known to be important for stability and that compose the chromophore^{41,42} (FIG. 3A-3C). When building the site-saturated library targeting these four residues (80 mutants), a high tolerance to primer design deviations (e.g., homologous overlaps, melting temperatures) was found (FIGS. 2A-C and 4A-C) and that LETs of muGFP variants conferred all desired mutations (FIG. 4D). Like others⁴⁰, it was shown that residues composing the fluorophore and impacting hydrophobic core packing were intolerable to mutations while a surface exposed residue facilitating hydrogen bond networks had less impact on green fluorescence under the conditions tested (FIGS. 3A-3C). Full-length soluble proteins indicated that changes in fluorescence were not due to changes in expression or solubility (FIGS. 4E-4F). Mapping the site-saturated landscape not only highlights residues that are crucial for fitness but also provides insight into the general mutability of sites. Importantly, our method can facilitate entirely in vitro, rapid design-build-test-learn cycles using ISM.

Performance of muGFP variants were quantified by measuring fluorescence on a plate reader (BioTek Synergy 2) using an excitation of 485 nm and emission of 528 nm. 10-μL of crude CFPS reaction containing an expressed muGFP variant was transferred to a Nunc black, round bottom 384-well plate prior to measurements.

### Example 3—Amide Synthetase Activity Assays

All high-throughput assays (hot spot screen, iterative site saturation mutagenesis, substrate scope, ML predictions validation, and ML prediction exploration) were assembled in 384-well plates (Bio-Rad) using an Integra VIAFLO liquid handling robot. A 2× reaction mix containing the substrates (ATP, acid, amine, and DMSO) with excess volume filled with 50 mM potassium phosphate pH 7.5 was dispensed as 3-μL aliquots in a 384-well plate. The amidation assay was initiated by adding 3-μL of crude CFPS reaction containing an expressed McbA variant, with final concentrations of 25 mM ATP, 25 mM acid, 25 mM amine, 10% v/v DMSO, and ˜1 μM of enzyme (determined by ¹⁴C-leucine incorporation using previously described protocols⁶⁶). Stock solutions of the acids were prepared in DMSO and this was taken into account to reach 10% v/v DMSO. For reactions that were performed in triplicates, 3-μL from the same 10-μL CFPS reaction was used for three separate assays. The reaction was incubated at 37° C. for 16 hours and then quenched with 25-μL of methanol. Plates were stored at −20° C. until prepared for analysis.

Amidation assays for the purified McbA variants were set up similarly as described above. 8-μL reactions were assembled in triplicate, containing 25 mM ATP, 25 mM acid, 25 mM ATP, 10 mM MgCl₂, 10 U/mL pyrophosphatase (Sigma I5907), 0.5 mg/mL McbA, 10% v/v DMSO, and volume to fill of 50 mM potassium phosphate pH 7.5. For assaying the production of cinchocaine and procainamide, substrates were decreased in stoichiometric amounts to 20 mM and 10 mM, respectively. This was to compensate for an observed poor solubility of these two acids (2-butoxyquinoline-4-carboxylic acid and 4-aminobenzoic acid) in the purified reaction at 10% v/v DMSO. Reactions were incubated at 37° C. for 16 hours and then quenched with 25-μL of methanol. The CAS numbers of all chemicals used in the hot spot screens, as well as the amide standards the inventors purchased, can be found in Table 1.

TABLE 1
CAS numbers of compounds used.

| Product | Acid | Amine | Amide |
|---|---|---|---|
| Moclobemide | 74-11-3 | 2038-03-1 | 71320-77-9 |
| Metoclopramide | 7206-70-4 | 100-36-7 | 364-62-5 |
| Cinchocaine | 10222-61-4 | 100-36-7 | 85-79-0 |
| Itopride | 93-07-2 | 20059-73-8 | 122892-31-3 |
| Declopramide | 2486-71-7 | 100-36-7 | 891-60-0 |
| Trimethobenzamide | 118-41-2 | 20059-73-8 | 554-92-7 |
| (S)-Sulpiride | 22117-85-7 | 22795-99-9 | 15676-16-1 |
| Procainamide | 150-13-0 | 100-36-7 | 51-06-9 |
| Troxipide | 118-41-2 | 334618-23-4 | 30751-05-4 |

The workflow was implemented in two sequential parts: (1) a hot spot screen (HSS) in which we perform site-saturated mutagenesis on a wide sequence space to identify residue positions that, when mutated, positively impact fitness, and then (2) ISM to accumulate beneficial combinations of mutations focused on impactful residue positions identified from the HSS. Importantly, our McbA engineering campaign was evaluated using high substrate concentrations in an effort to reach more industrially relevant reaction conditions (FIG. 5A). Guided by the crystal structure of McbA (PDB: 6SQ8), 64 residues that completely enclosed the active site and putative substrate tunnels were selected. The HSS of these residues (1,280 total single mutants) revealed six that had a positive impact on moclobemide synthesis when mutated compared to wild-type McbA (wt-McbA) as measured by LC-MS (FIG. 5B). After fixing the top mutation from the HSS (V177S), ISM was performed on the remaining five residues identified in the HSS over three rounds (FIG. 5C). Notably, the workflow reintroduces previously fixed mutations to explore potential epistatic interactions (e.g., S177 was saturated in ISM step 2, given V177S was incorporated before A323F). In addition, exhaustive exploration of combinatorial double mutants of the top two residues showed directly additive impacts for moclobemide synthesis (FIG. 5D).

After round three of our ISM workflow, we identified a quadruple mutant (qm-McbA_{moc}) with dramatically increased activity for the synthesis of moclobemide (FIGS. 5E-5F). Under the final screening conditions, an increase in yield was observed from 12% to 96% conversion from wt-McbA to qm-McbA_{moc}, respectively. The apparent steady-state kinetic parameters and stability of the top-performing enzyme mutants from each round of ISM were then characterized. each McbA variant was expressed, purified, and evaluated, and a 42-fold increase was observed in catalytic efficiency from wt-McbA to qm-Mcba_{moc} (k_{cat}/K_{M} increased from 18.2 to 769 M⁻¹ min⁻¹) for the amine (FIG. 5F and FIGS. 7A-7B). Next the melting point of the variants was measured using circular dichroism (CD) to evaluate potential stability changes (FIG. 8). Interestingly, the melting point did not significantly change between wt-McbA and qm-McbA_{moc}, but the second mutation (A323F) increased Tₘ by 5.81±0.09° C. when added to the first mutation (V177S).

Toward evaluating McbA's use as an industrial biocatalyst for moclobemide production, an ATP recycling module was implemented using polyphosphate, a polyphosphate kinase, and catalytic starting quantities of AMP in place of ATP in our reaction conditions (FIG. 6A)^{35,43}. A yield of 53% was obtained when using just 0.19 mM of AMP (0.0076 equiv.). Then, the reaction was scaled 1,000-fold of our screening conditions to make milligram quantities of moclobemide confirmed by NMR (FIG. 6B). Further optimization (e.g., increased enzyme loading, batch or continuous feeding of substrate or enzyme, enzyme immobilization, etc.), could enable higher yields.

### Example 4—Modified Amide Synthetases “McbA” Marinasctinospora thermotolerans Enzymes

### Diversifying the Biocatalytic Synthesis of Amides

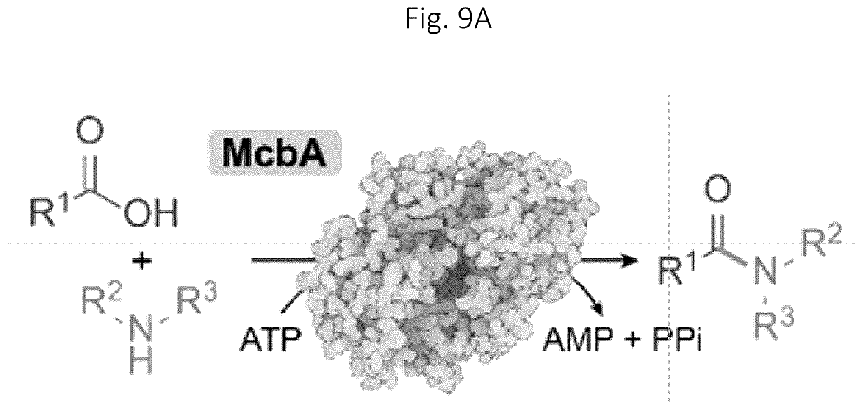

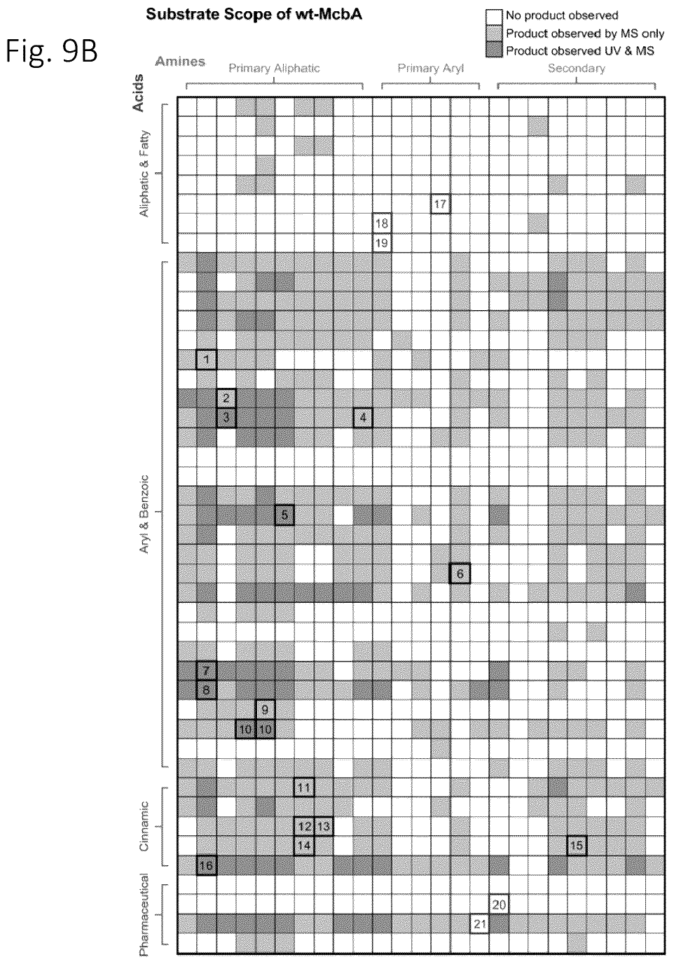

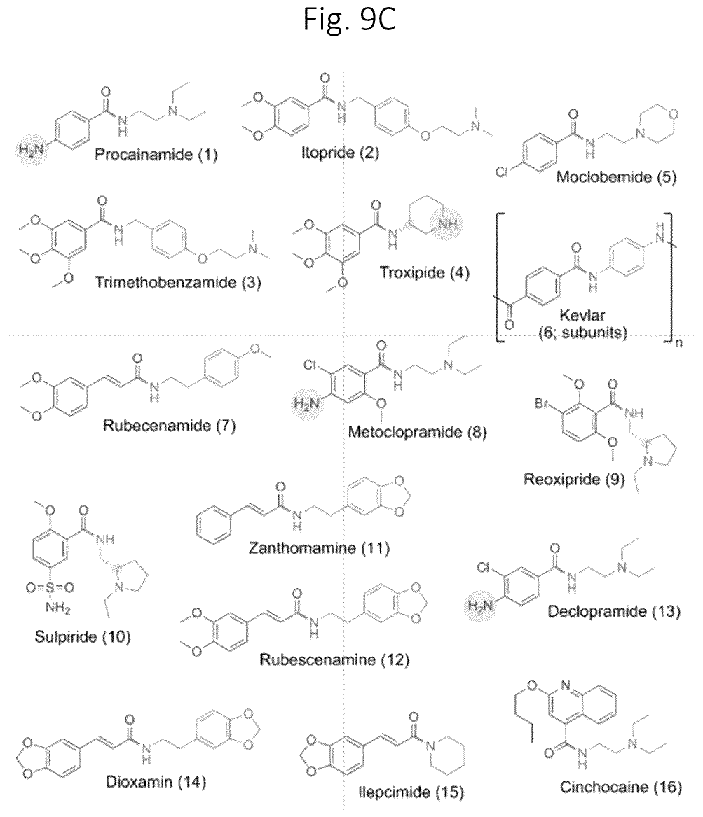

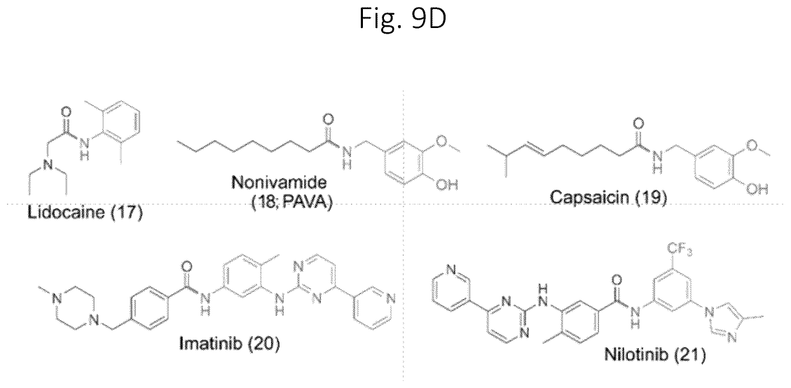

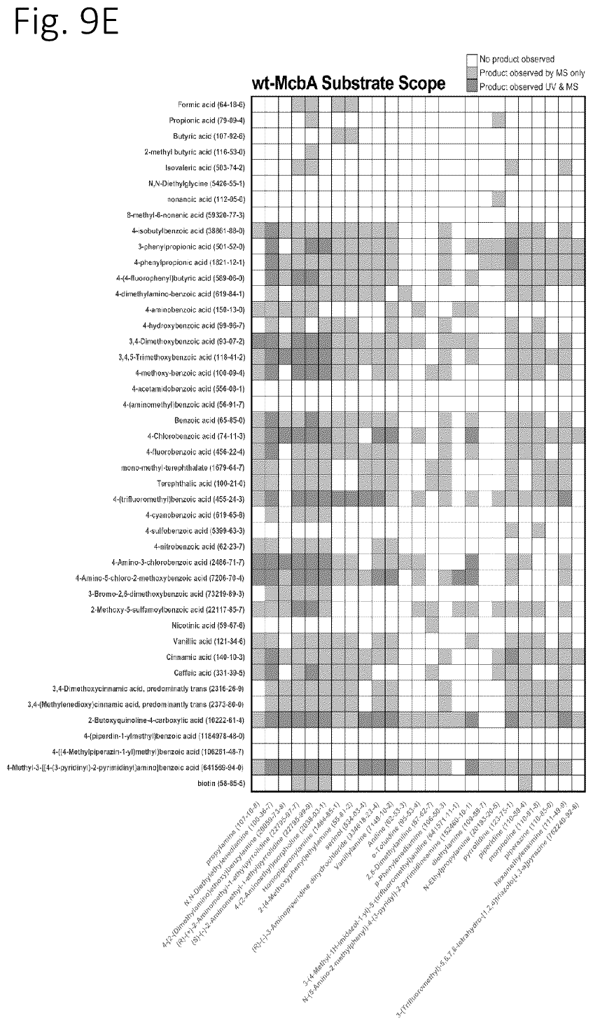

Having established the engineerability of McbA for moclobemide, the possible enzymatic amidation reaction space of McbA was further explored by performing an extensive substrate scope screen of a diverse variety of acids and amines (FIG. 9A), with screening conditions of 25 mM acid, 25 mM amine, 1 μm CSL-McbA, 10% DMSO, and 50 mM K₂HPO₄ buffer, at a pH 7.5-8.0. With this intent, our substrates largely deviated from the heterocyclic acids and primary or aromatic amines preferred by McbA and included primary, secondary, alkyl, aromatic, complex pharmacophore, electron poor or rich species, and substrates containing other heteroatoms, halogens, and “unprotected” nucleophile or electrophile representatives. More challenging substrates (e.g., complex heterocyclic acids and amines, enantiomers, and substrates containing both acids and amines or multiple acids and amines) were included to determine the innate limitations and preferences of McbA. In total, 1100 unique reactions were explored (FIG. 9B) covering 21 molecules of known value including pharmaceuticals, fragrances, polymers, and non-lethal-munitions. These reactions were performed under challenging conditions—enzyme concentration was kept low at 0.05 mg/mL (˜1 μM), substrate concentrations were relatively high at 25 mM (equimolar acid, amine, and ATP), and the final reaction contained 10% DMSO v/v. We reasoned these conditions may highlight reactions McbA would be most robust to and, potentially, the most evolvable towards.

Overall, McbA displays a remarkably relaxed substrate scope with innate chemo-, regio-, and stereoselectivities that could be exploited through enzyme engineering. McbA, is highly tolerant to a wealth of “unprotected” functional groups and geometries and was able to synthesize 16 of the 21 high-value compounds, 11 of which are small-molecule pharmaceuticals (FIG. 9C). Generally, aliphatic and alkyl acids were poorly tolerated while aryl, benzoic, and cinnamic acids were readily accepted substrates. Charged aryl acids were a unique exception and usually coupled to very few amines. Conversely, wt-McbA readily coupled primary and secondary aliphatic amines but struggled with aryl amines. Surprisingly, only 6/44 acids failed to yield any product while every single amine could be coupled to at least one acid, suggesting McbA has a more strict acid substrate scope. McbA was able to synthesize several pharmaceutical compounds as well as dozens hybrid molecules, ranging from trace amounts detectable only by MSD to approximately 12% conversion. In these reactions, both stereoselectivity (e.g., strongly favoring the synthesis of S-sulpiride over R-sulpiride) and strict chemo-and regioselectivity (e.g., substrates containing acids and amines not polymerizing) preferences were uncovered. Given that the reaction mechanism of McbA first begins with the adenylation of the carboxylic acid, we also noticed several instances where only the acyl-AMP intermediate was observed. This finding agrees with recent work observing a more relaxed substrate preference for the acid over the amine^{35,36}. Interestingly, several amide products containing amino acids were also found, as these are present in excess during CFPS of McbA. These data were unexpected but clearly demonstrate an even larger, more peptide-like chemical space could be accessed, similar to recently discovered ligases⁴⁴.

With the impressive breadth of the chemical space accessible with McbA, two additional protein engineering campaigns were engaged. Metoclopramide (3% wt conversion) and cinchocaine (2% wt conversion) stood out as interesting testing cases for several reasons. For instance, the acid component in metoclopramide contains an (unprotected) amine that could potentially compete with the intended amine and even polymerize; however, no such side reactions were observed and only metoclopramide was produced (compared to an authentic standard). Engineering regioselective biocatalysts are attractive because they can streamline syntheses. Cinchocaine shares the same amide fragment with metoclopramide but contains a unique acid fragment. By performing both engineering campaigns in parallel, mutations that influence substrate specificity for the amine (shared mutations) and the acid (unique mutations) may be inferred, which may lead to general design principles for McbA.

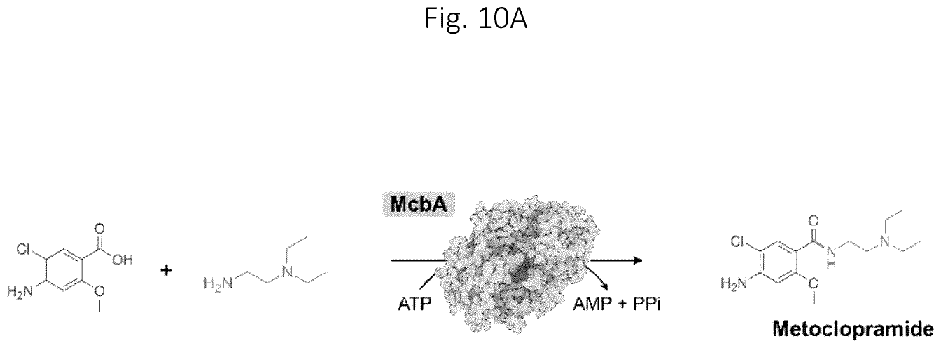

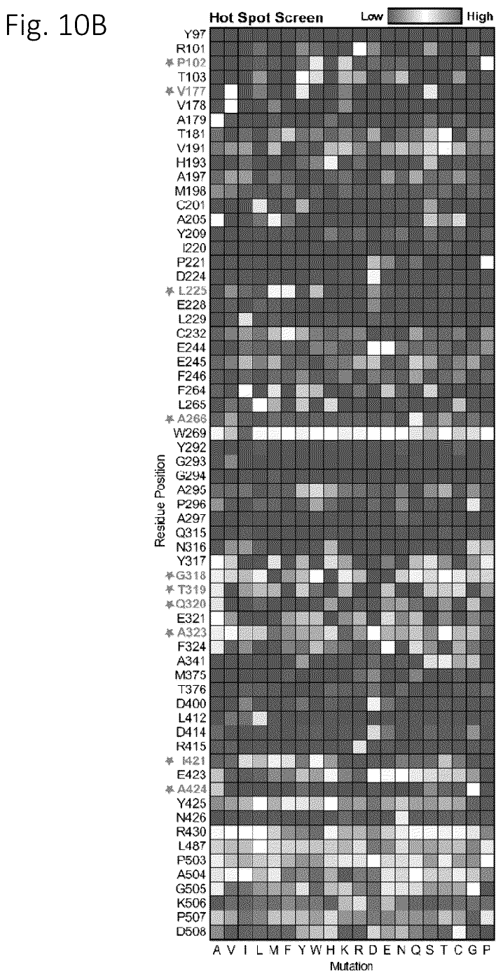

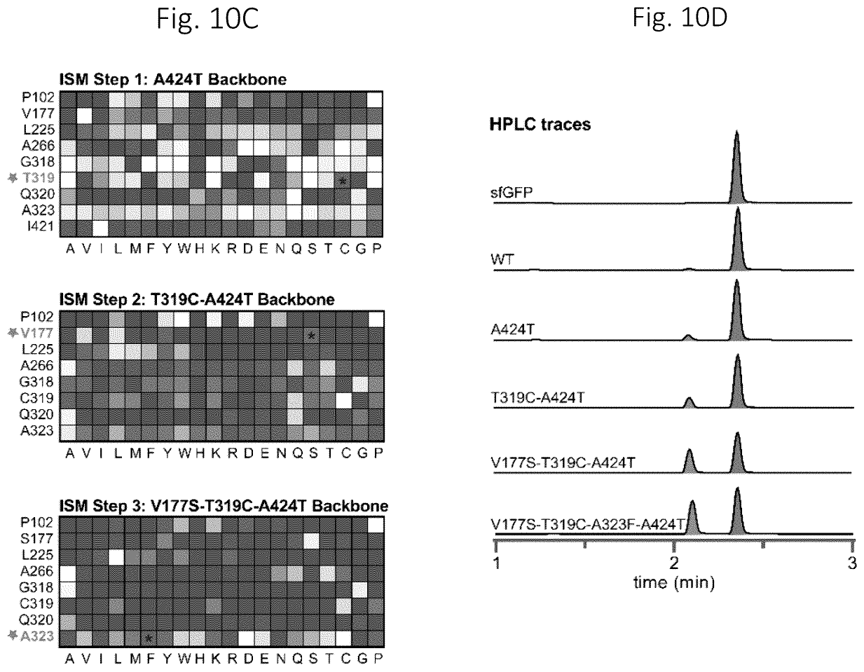

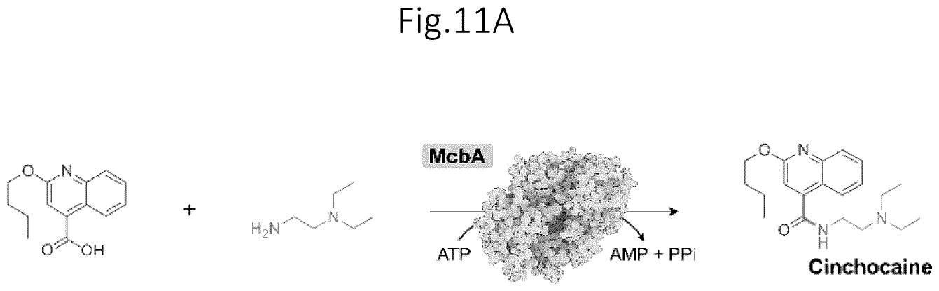

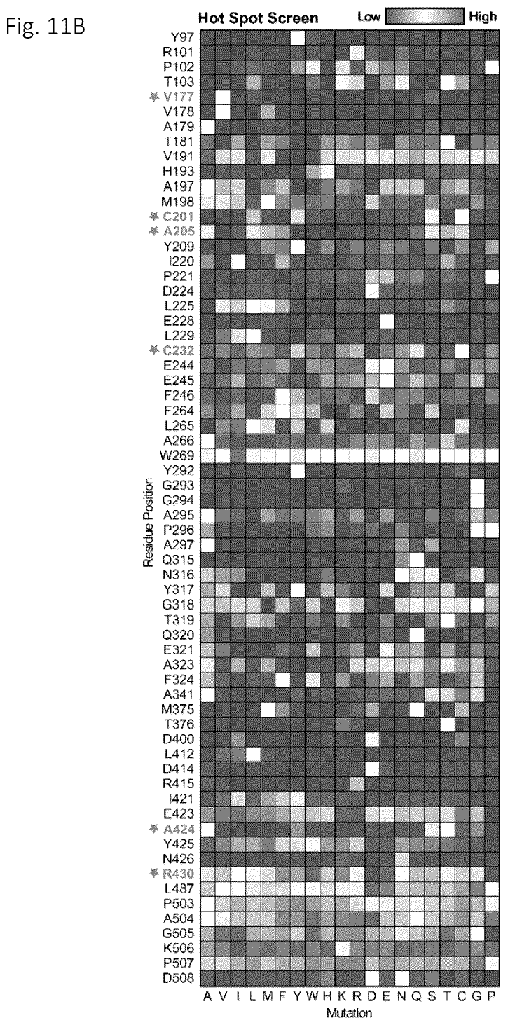

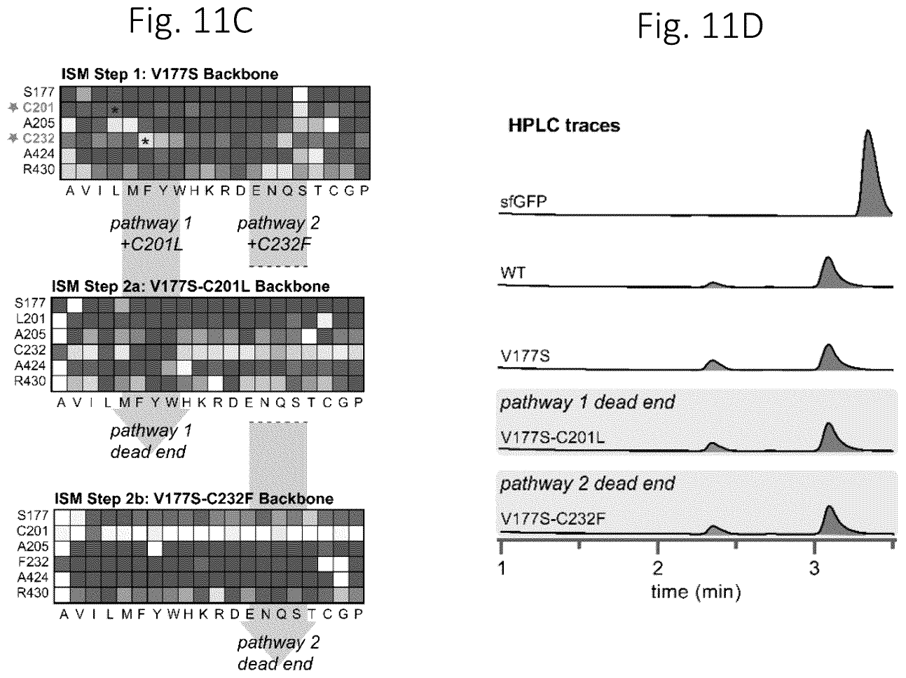

The HSS and ISM strategy was employed to identify unique McbA mutants for improved synthesis of metoclopramide (FIGS. 10A-10D) and cinchocaine (FIGS. 11A-11D). The HSS was performed on the same 64 residue positions identified for the moclobemide screen followed by ISM on a down-selected set. The HSSs revealed ten hot spot positions for metoclopramide (FIG. 10B) and six for cinchocaine (FIG. 11B). While only two hot spot positions are shared between metoclopramide and cinchocaine, both had the same top-performing mutations (V177S and A424T). A424 is predicted to be in the amine binding pocket while V177 is towards the end of the acid binding pocket. Surprisingly, V177S is a high fitness variant for all three compounds tested despite each containing a different acid component. Three rounds of ISM for metoclopramide yielded a quadruple mutant that displayed nearly 30-fold activity over wt-McbA (FIGS. 10C, 10D). The ISM path for cinchocaine was more difficult to navigate and beneficial mutations beyond a double mutant (˜1.5-fold activity over wt-McbA under our screening conditions) was not observed, despite taking multiple ISM paths (FIGS. 11C, 11D). After one additional engineering round (pathway 1), an additional beneficial mutation beyond the double mutant was not observed (FIG. 11C). Notably, mutations that were previously observed to be beneficial were no longer in future rounds. In an attempt to overcome this dead end, an additional engineering round using a double mutant consisting of the two best mutations found in the HSS was performed (pathway 2), and no additional mutations were found for this backbone, resulting in another dead end (FIG. 11D). The sequential nature of ISM can miss out on beneficial combinations that may arise from epistatic interactions. Integration of machine-learning models into the workflow could take advantage of the rich sequence-fitness landscape that was rapidly generated in the HSS and overcome the path dependency of the current approach.

### A Simple, Rapid, and Effective Strategy for Machine Learning-Guided Protein Engineering

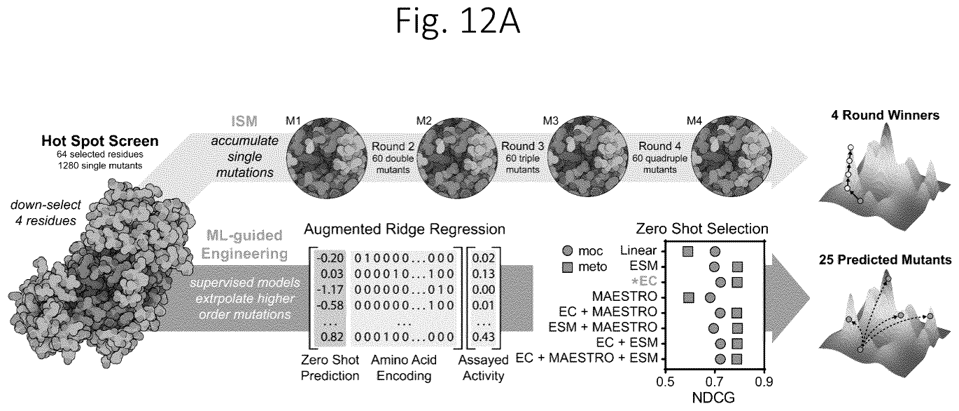

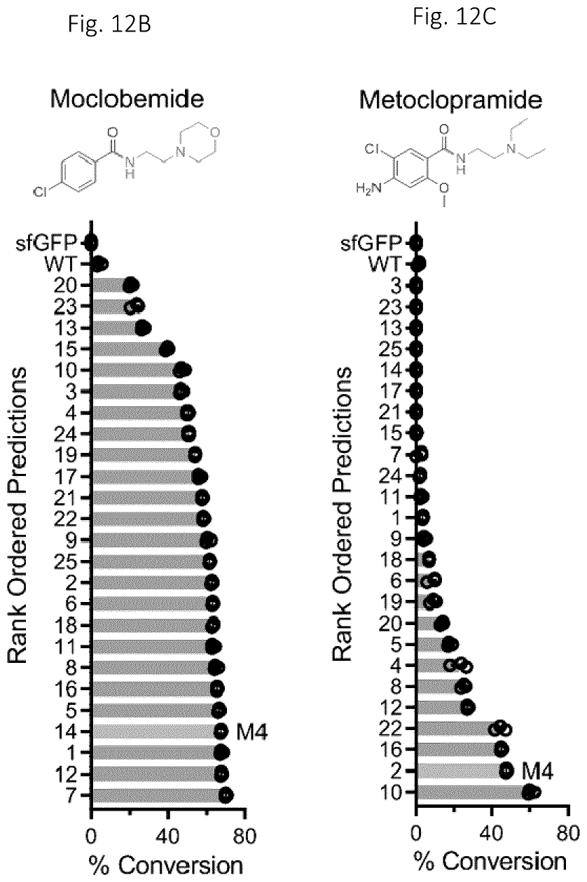

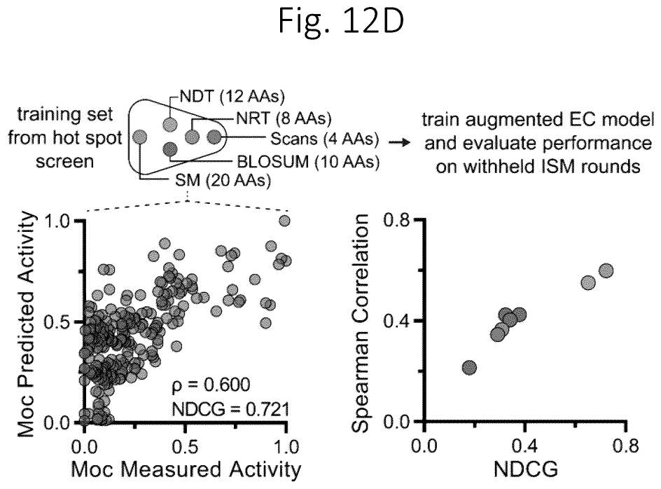

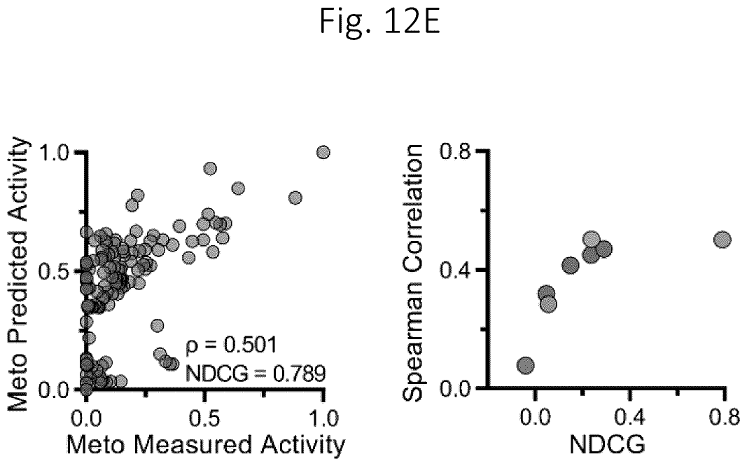

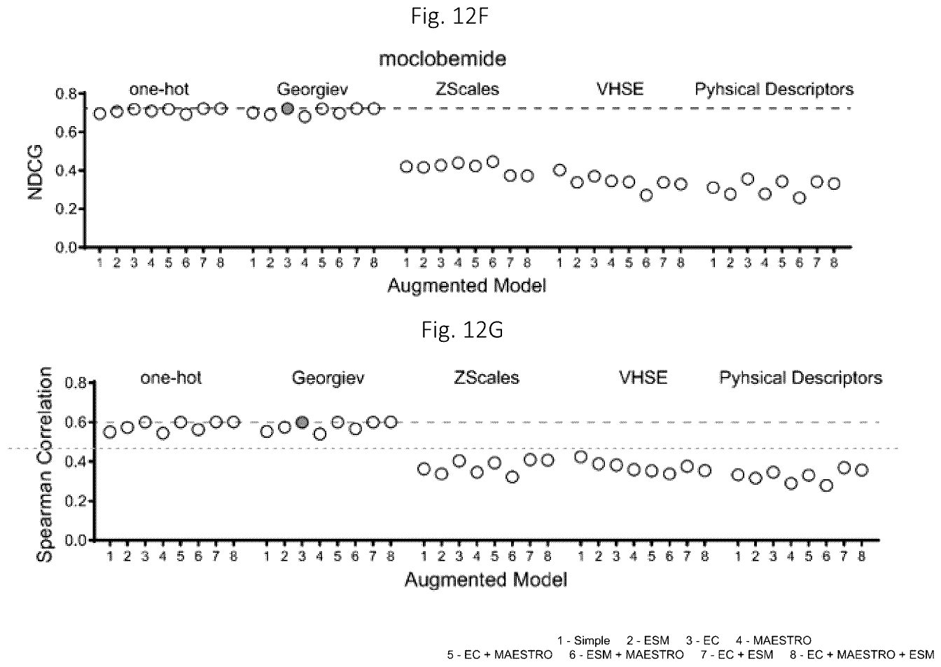

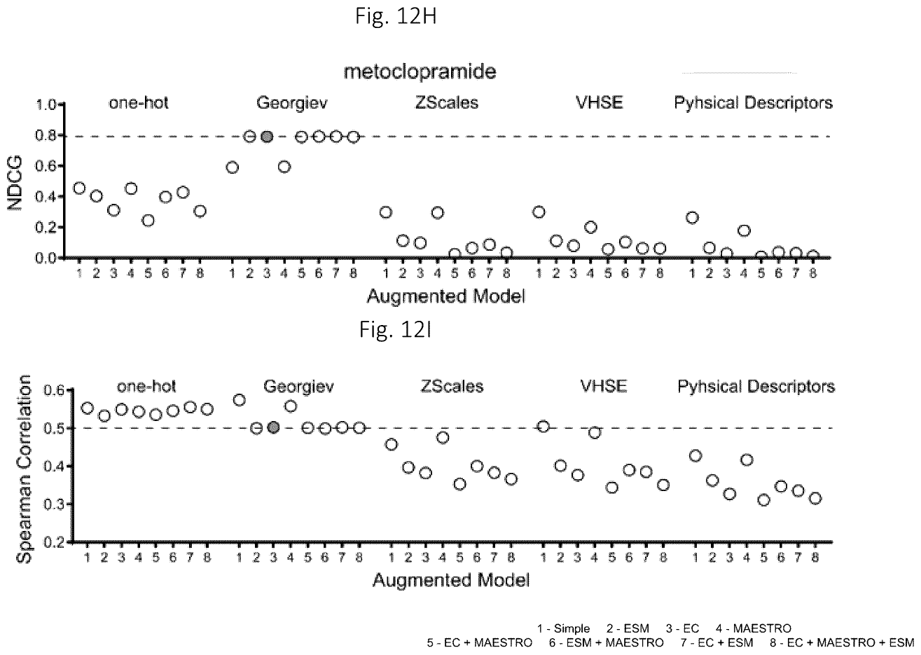

Machine learning methods were integrated into the workflow to overcome path dependencies, reduce the screening burden, and begin taking larger steps in sequence space. The disclosed machine-learning guided strategy would rely solely on our single mutants derived from the HSS to fit supervised regression models and extrapolate to higher order mutants with increased activity (FIG. 12A). This new strategy was first validated on moclobemide and metoclopramide synthesis. McbA variant feature representations consisted of site-specific amino acid encodings concatenated with a zero-shot fitness prediction²⁶. Here, several amino acid encodings were considered, ranging from simple one-hot encodings to more complex descriptors that attempt to incorporate amino acid physiochemical properties⁴⁵⁻⁴⁸, and explored benchmark protein variant fitness predictors to incorporate universal, evolutionary, and structural based predictions. We tested three fitness predictors in particular: the Evolutionary Scale Modeling (ESM)-1b transformer¹³ trained on the UniRef⁵⁰ database (universal), an EVmutation²² probability density model trained on an MSA of evolutionarily related sequences (evolutionary), and MAESTRO⁴⁹ to estimate structure-based changes in unfolding free energy (structural). Training and hyperparameter tuning were performed using single mutant data (n=77) from the HSS (top four hot spots; FIG. 5B and FIGS. 10A-10D), with each model being tested on the withheld ISM rounds containing double, triple, and quadruple mutants (n=243 for moclobemide and n=169 for metoclopramide). Model prediction performance was evaluated using the normalized discounted cumulative gain (NDCG)^{16,50}, an evaluation metric that scores models on their ability to correctly rank high-fitness variants, which generally matched results from the Spearman rank correlation coefficient (FIG. 12E). The augmented models we tested generally outperformed the ridge regression model alone, with similar trends observed for both compounds. We also tried combining predictors in our regression features (e.g., predictions from both ESM-1b and EVmutation), but no increase in model performance was gained. Moving forward, we decided to use the augmented EVmutation model with Georgiev encodings given the strong predictive performance among both compounds and the already-trained probability density model greatly simplified application to other compounds.

The augmented model was used our to screen 20⁴ combinatorial enzyme variants in silico and selected the top 25 predictions to subsequently test experimentally. The augmented model was found to be able to predict McbA variants enriched in high activity when tested experimentally, some even surpassing qm-McbA from both moclobemide and metoclopramide campaigns (FIG. 12B-12C). Notably, the best predicted mutant for metoclopramide contained a mutation (A424S) that was superseded in the HSS by a more active mutation (A424T) carried forward in ISM, indicating the model found a superior mutant that would have been overlooked using traditional directed evolution strategies. Given that the EVmutation probability density model was trained on evolutionary related sequences to McbA, how the augmented model improved predictions of variants catalyzing unnatural reactions was surprising. However, it is possible that the permissible sequence space for McbA is evolutionarily constrained, and the model is helping navigate to areas that allow for the desired reaction to occur while still leading to a folding, functional protein. It is likely there are instances where this zero-shot predictor would not be amenable to engineer an enzyme towards unnatural substrates.

Whether the entire site saturation dataset was necessary to train models that had high predictive performance was tested. Variants in the training set were deliberately withheld to reflect common protein engineering strategies that attempt to adequately sample amino acids without exhaustively searching the sequence space. These included reduced codon libraries (NDT⁵¹ and NRT⁵²) and scans⁵³ (here, we combine the commonly used glycine, alanine, proline, and cysteine scans). We also included an additional four reduced alphabets based on correlations in the BLOSUM50 similarity matrix which, unlike the codon libraries, naturally group amino acids by physiochemical properties as opposed to ease of experimental implementation⁵⁴. When training the same augmented ridge regression model with Georgiev encodings, this retrospective analysis indicated that utilizing all the data gathered in SM provides far more predictive power (FIG. 12D-12E). This can likely be attributed to the nature of the rich site saturation datasets mostly containing mutants with non-zero activity ( 64/77 for moclobemide and 62/77 for metoclopramide). This agrees with previous reports describing the importance of preventing “holes” in training sets¹⁶.

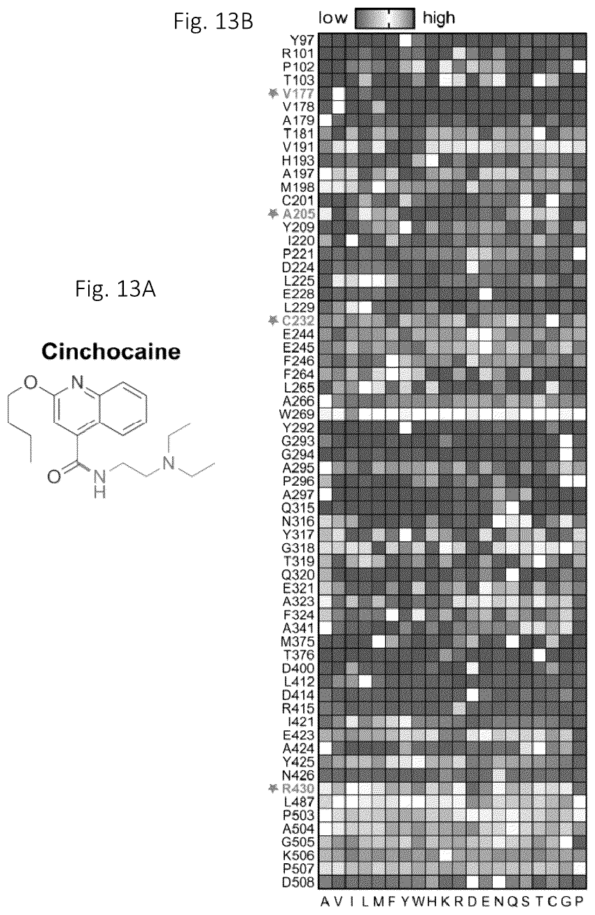

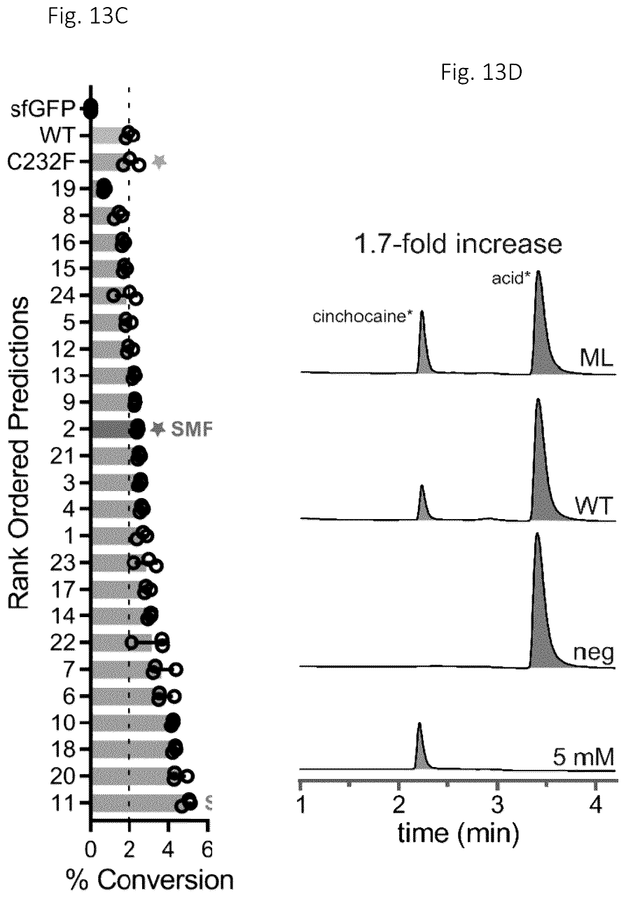

Having a predictive model architecture in place, we next targeted cinchocaine given our difficulty of improving this reaction using standard ISM (running into “dead ends”; FIGS. 11C-11D). Analysis of the tested model predictions revealed several interesting findings (FIG. 13A-13D). First, like our earlier results, the model predicted several variants with increased activity over wt-McbA. Second, the best predicted variant surprisingly contained a mutation (A205L) that decreased activity compared to wt-McbA in the HSS; we could not rationally select and combine mutations from the HSS to reach the same results. Third, the top predicted mutant had significantly higher activity than the best single mutation on its own. These results reiterated that our machine-learning guided strategy has the capacity to greatly improve our ability to rapidly discover high fitness variants for a variety of molecules using the same starting enzyme while avoiding path dependencies and reducing the screening burden.

### Machine-Learning Guided Biocatalytic Diversification for High-Value Pharmaceuticals

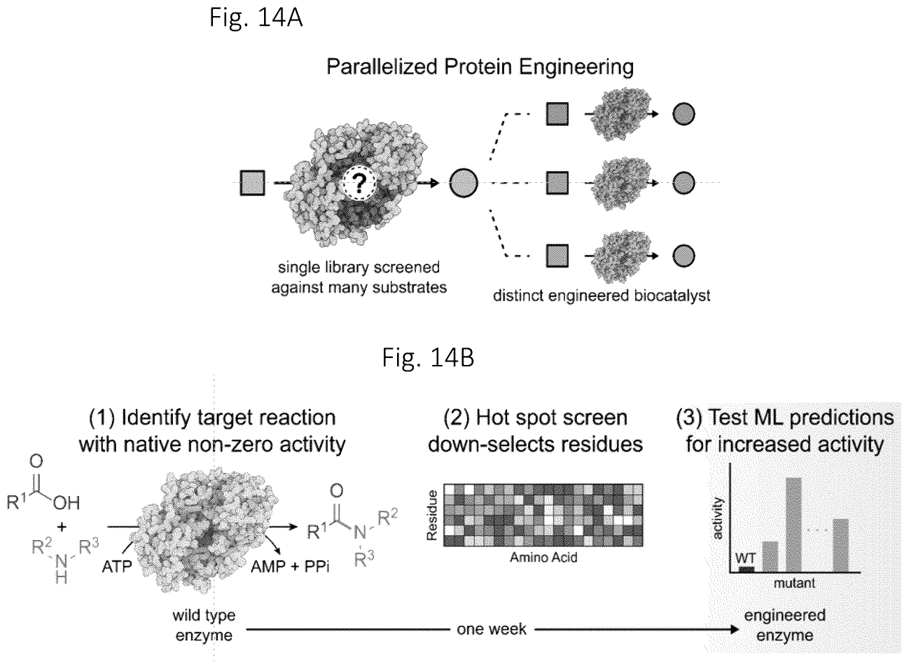

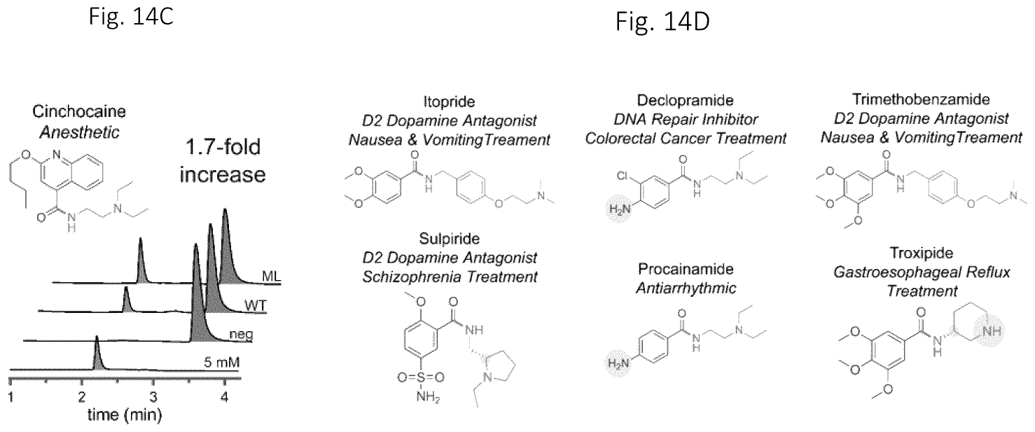

With the MLDE strategy validated, the approach could enable parallelized protein engineering for a wide variety of molecules (FIG. 14A). Starting with an identified target reaction from our substrate scope screen (FIGS. 5A-5F), we could use the same instance of our 1,280 single mutant McbA variant library to perform an HSS, select four hot spots, train and implement our machine learning model, and experimentally test the top 24 predictions (FIG. 14B). This approach was used to engineer distinct McbA mutants for the synthesis of an additional six pharmaceutical compounds (FIGS. 14B-14C). After identifying the best predicted variant for each reaction, the best variant for each reaction was expressed and purified, and the activity for each compound compared to wt-McbA (FIG. 14D). Fold-increase in yield observed compares wt-McbA to ML-McbA (n=3) for each small-molecule pharmaceutical. Increases in yield of the best mutant were observed ranging from 1.6-fold to 34-fold over wt-McbA for the six compounds tested.

| Compound | Fold-Increase |
|---|---|
| Itopride | 1.6 |
| Cinchocaine | 1.7 |
| Declopramide | 2.7 |
| Trimethobenzamide | 4.1 |
| Sulpiride | 6 |
| Procainamide | 10 |
| Troxipide | 34 |

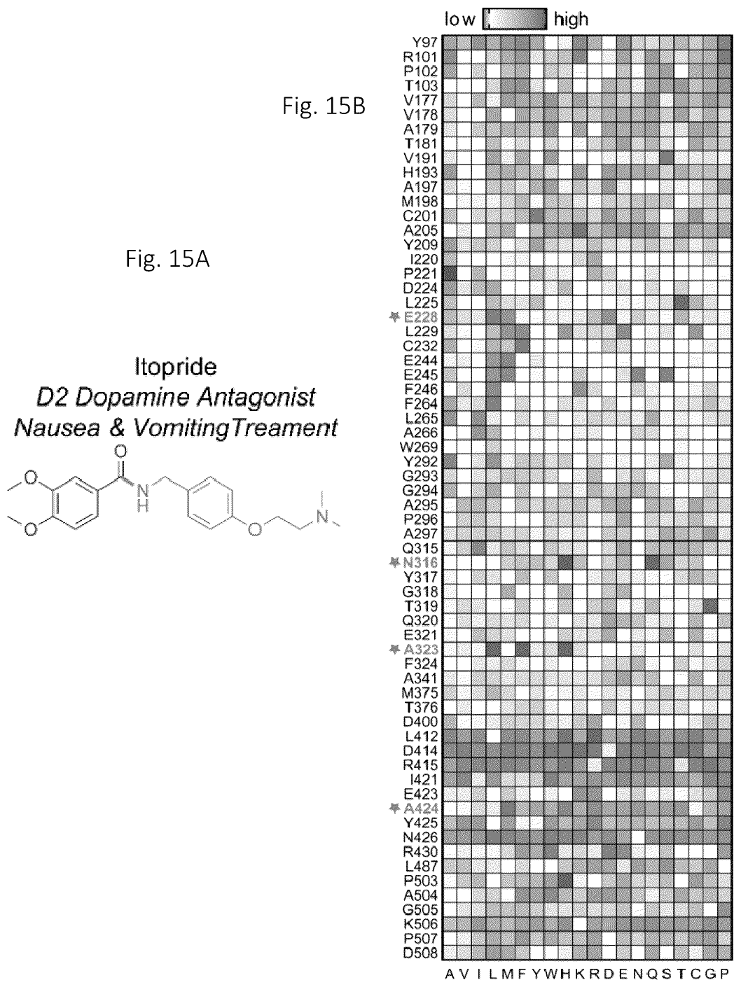

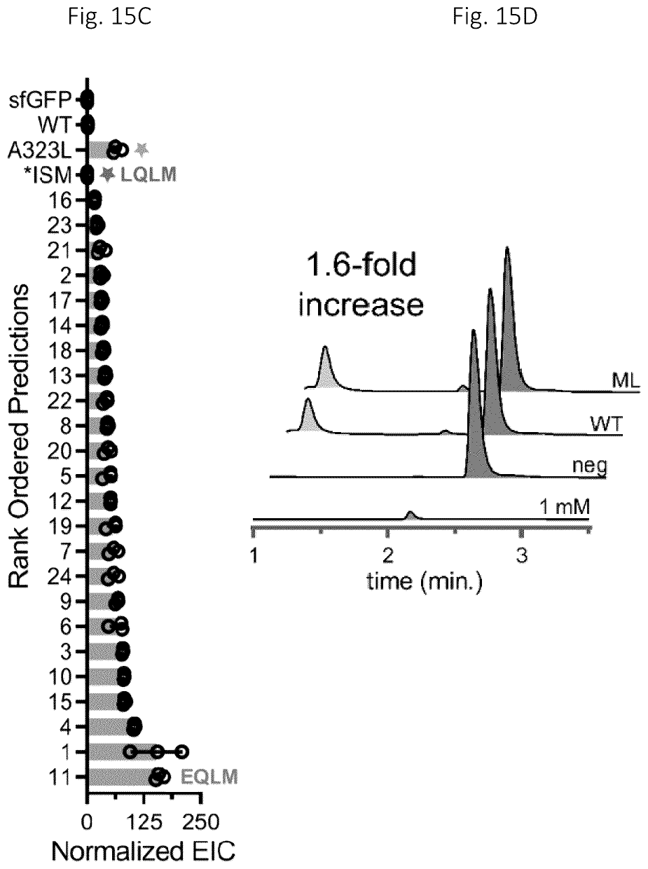

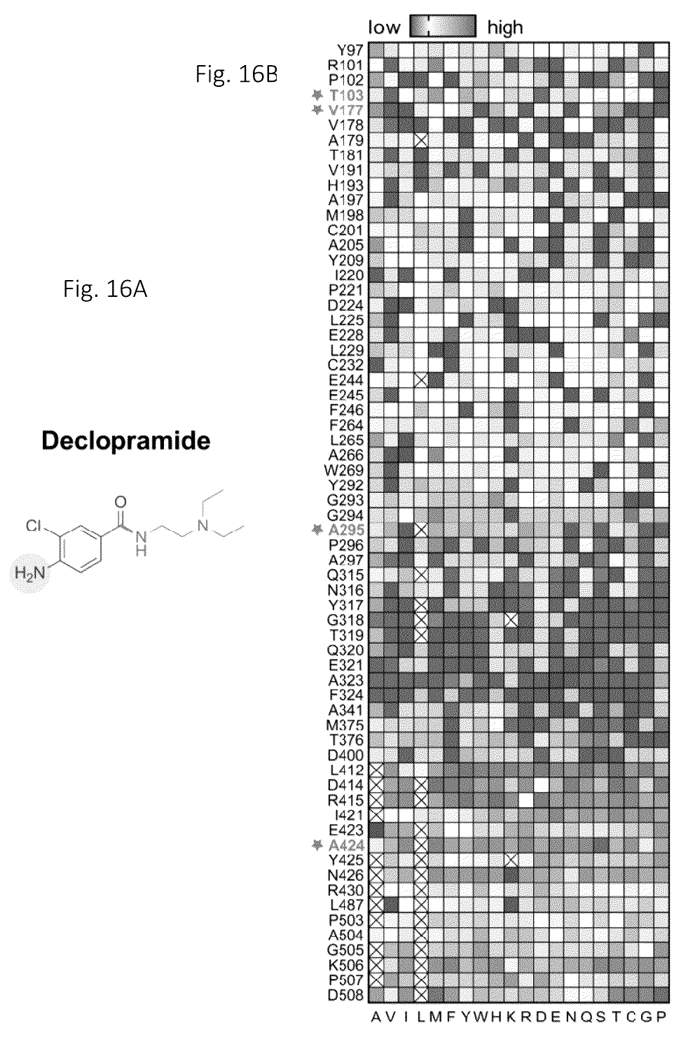

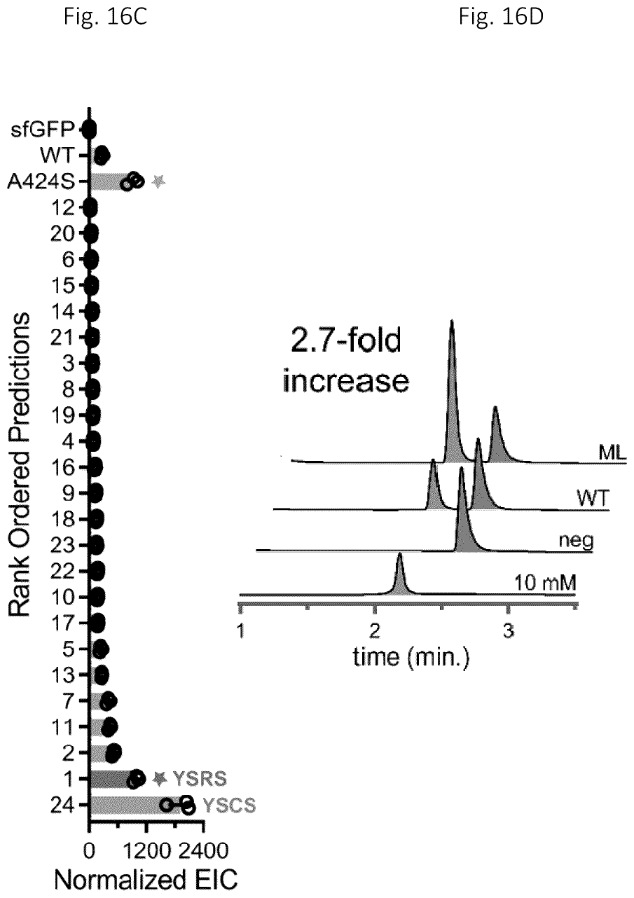

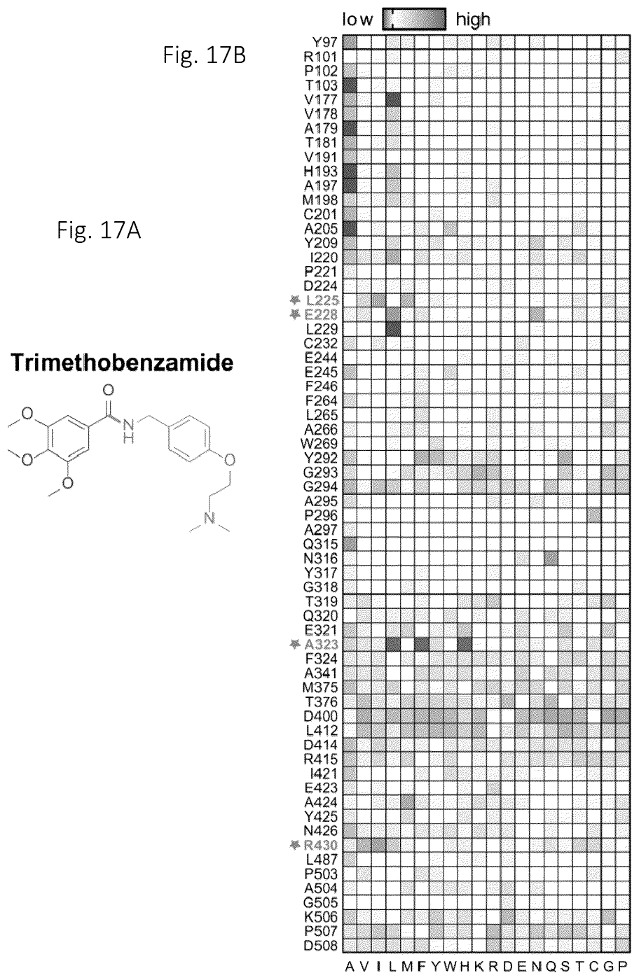

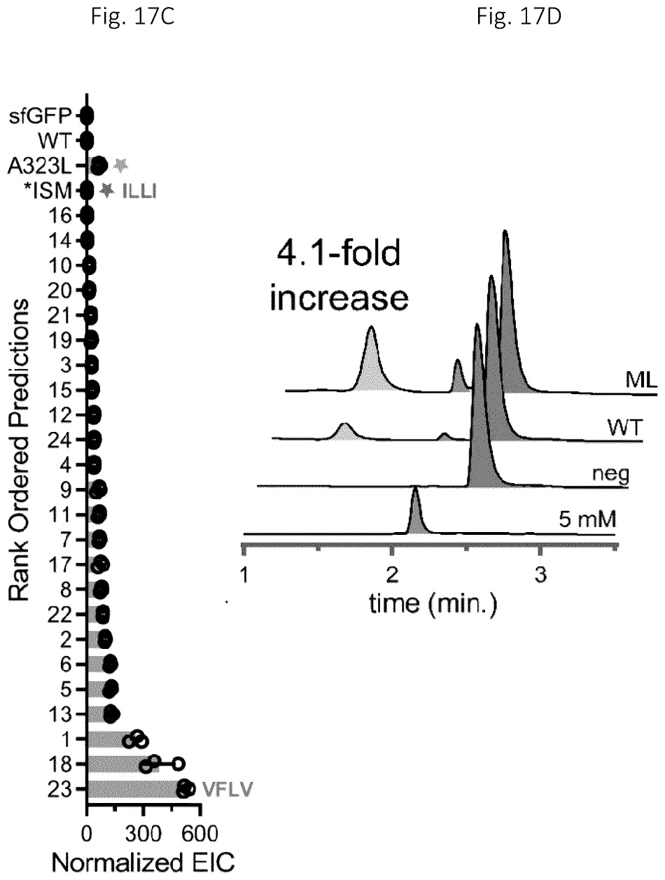

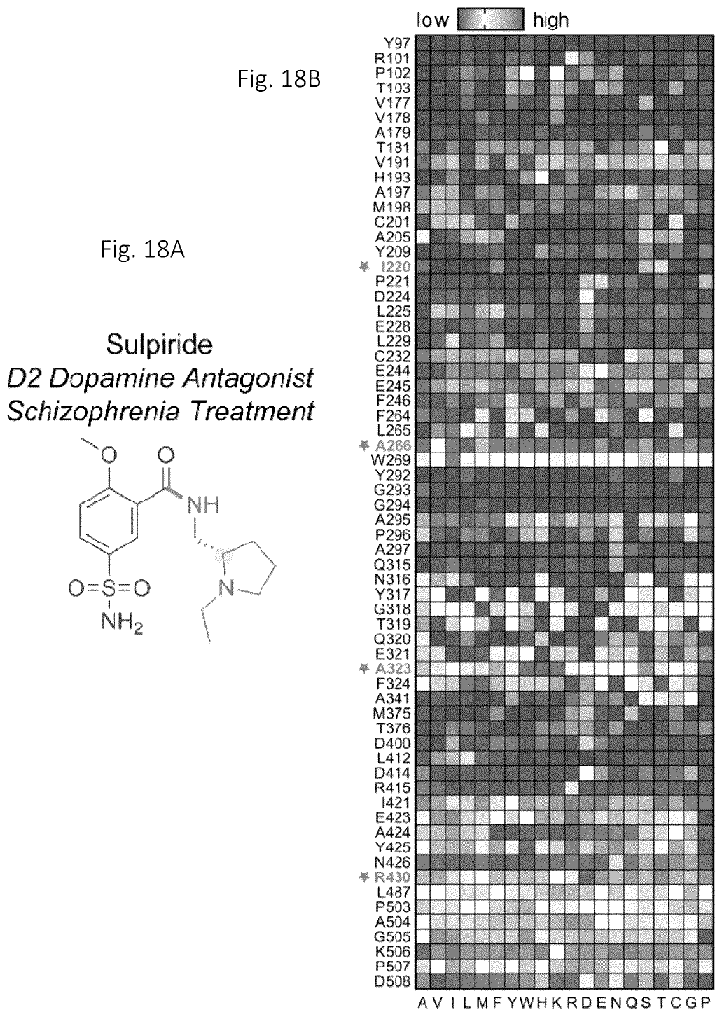

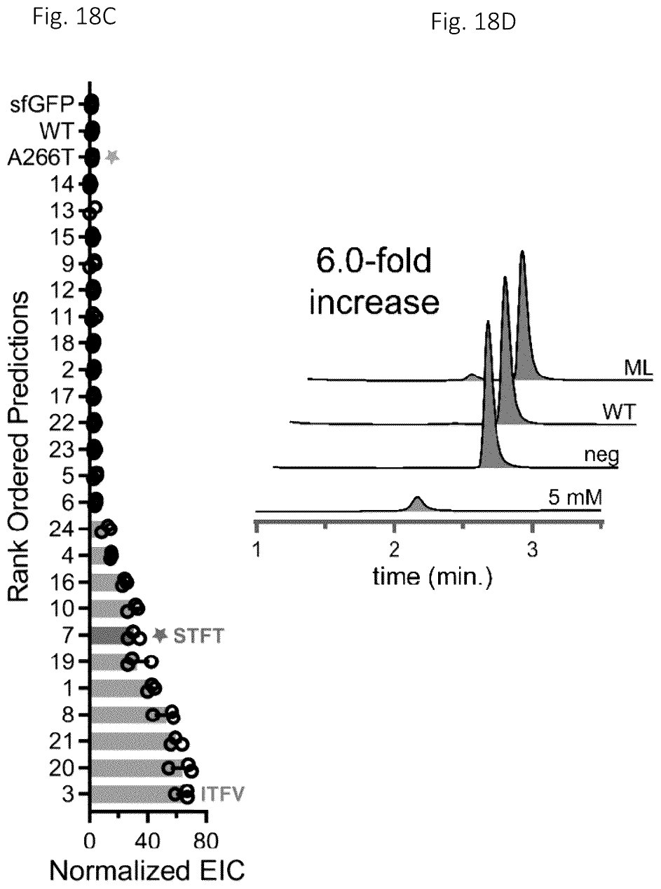

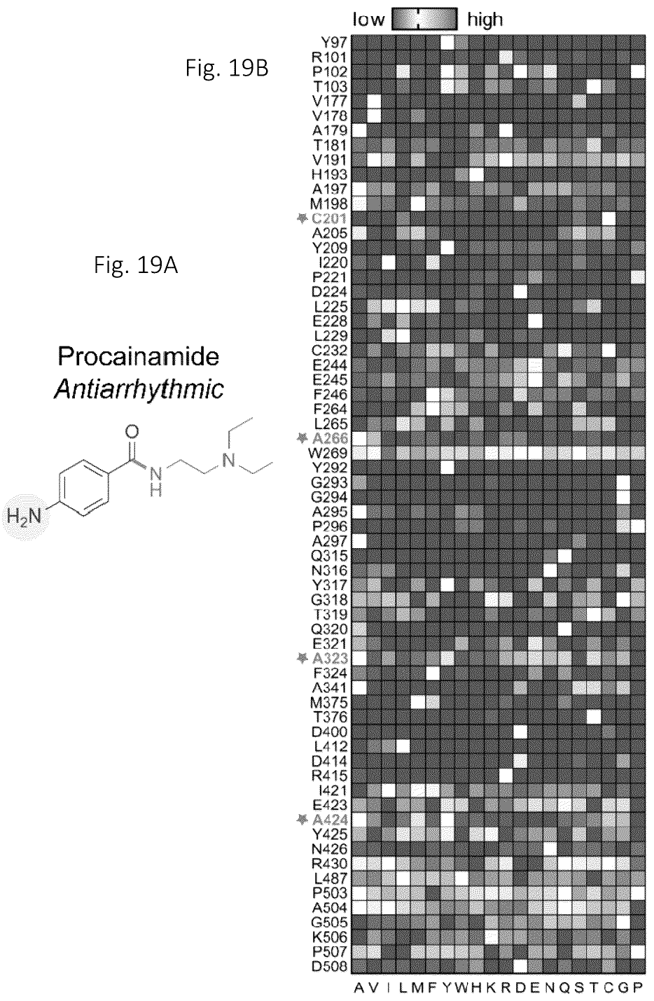

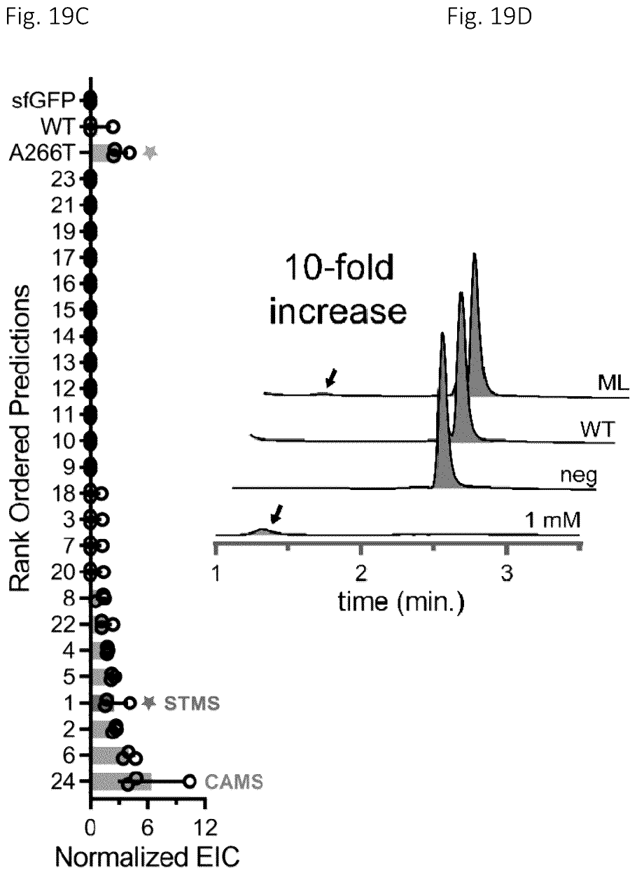

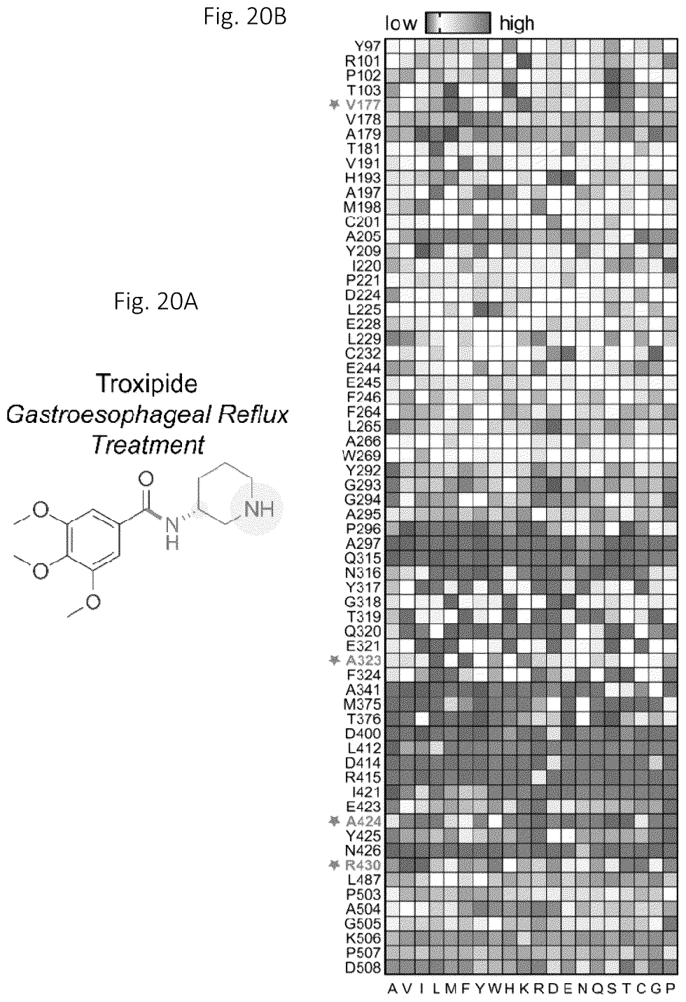

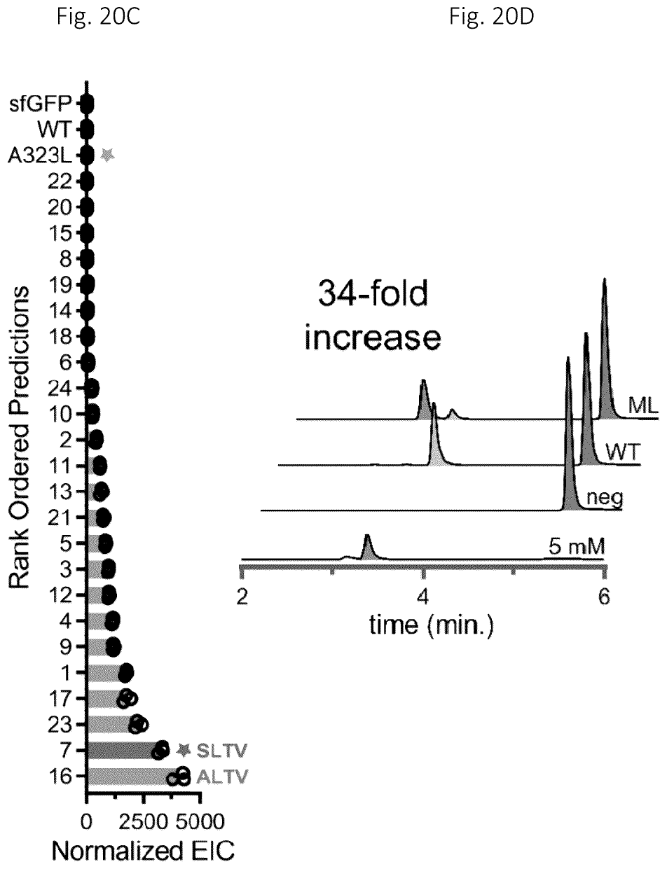

For each compound, the best predicted mutant always outperformed the best semi-rational design (combining the four best mutations from the HSS) (FIGS. 15A through 20D). While some mutants appear to give only subtle improvements, this may be an artifact of low signal-to-noise in the hot spot screens for some of the target compounds. Products that were only detectable by MS have a higher chance of error and fewer non-zero fitness mutants since even minorly defective mutants may produce product concentrations below the limit of detection. This ultimately leads to flat fitness landscapes that are more difficult to model. Despite these challenges, the model's and overall strategy yielded enzyme mutants with increased activity for multiple products that were initially only observed in trace amounts by MS. As enzymes improve and more robust datasets are generated the predictive power and the ability to navigate sequence space will strengthen (as seen with moclobemide and metoclopramide).

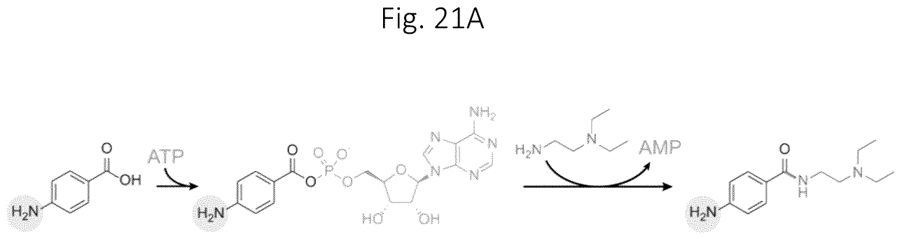

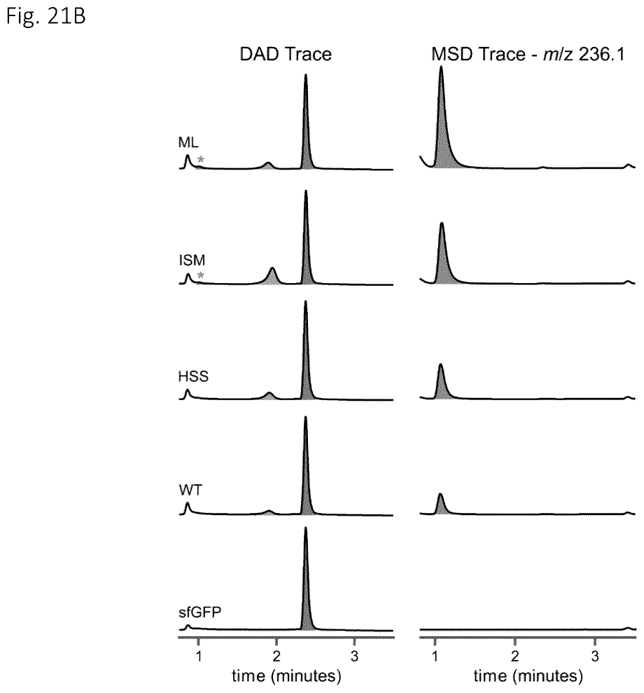

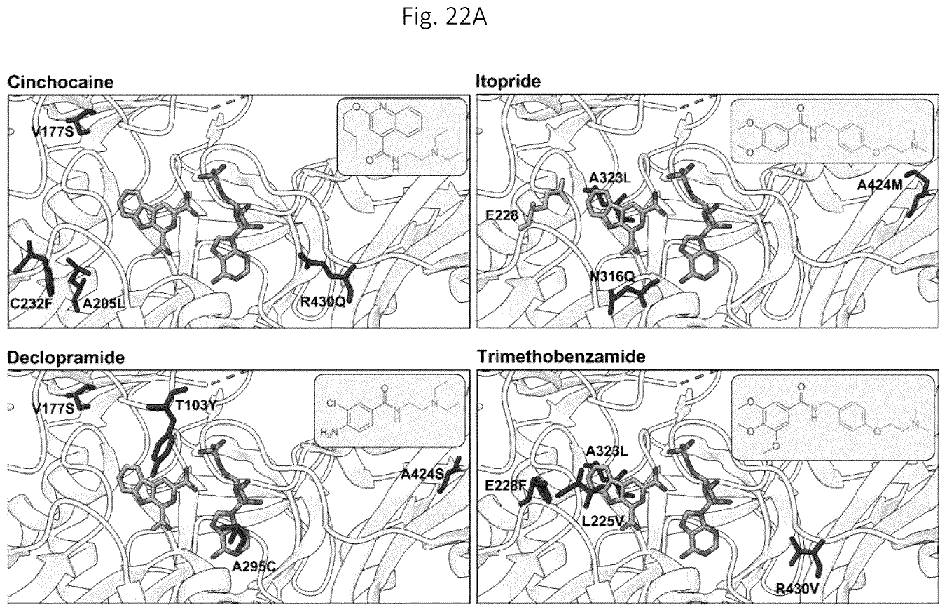

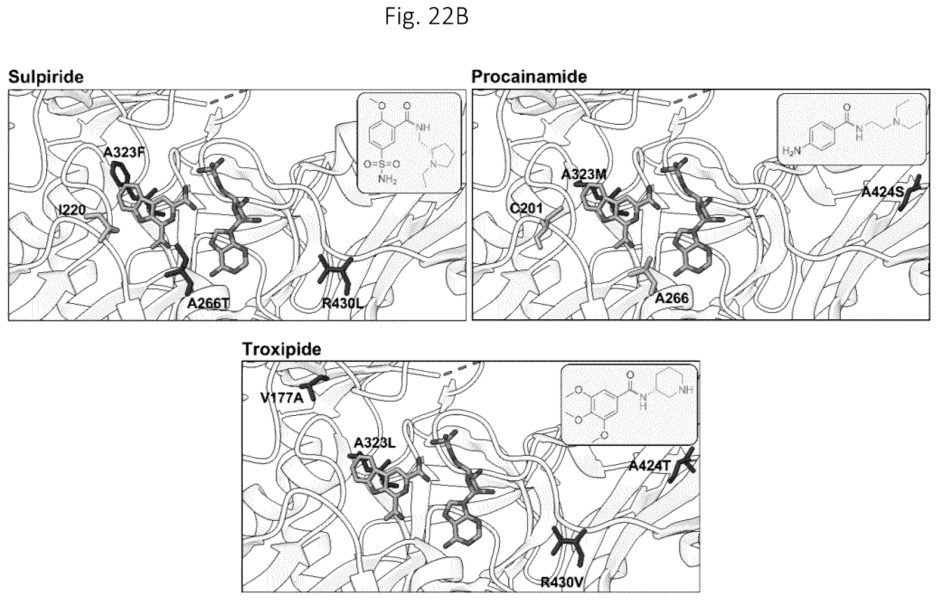

How efficiently some enzyme variants perform each reaction step (acid adenylation and amide bond formation) can also be compared (FIGS. 21A-22B). For example, wt-McbA appears to be proficient at the adenylation step for troxipide (adenylating 3,4,5-trimethoxybenzoic acid), but unable to catalyze amide bond formation. The engineered enzyme variant can subsequently accept the amine, leading to a large decrease in the observed intermediate. Similar to metoclopramide, McbA was able to catalyze the formation of procainamide, declopramide, and troxipide with the desired regioselectivity and no evidence of side products or polymerization. Serendipitously, the engineered McbA variants for each target product also displayed this strict regioselectivity despite the lack of any selective pressure to maintain it, meaning they aren't just more active, but more specific as well. This is exemplified by the quadruple mutant for troxipide that exhibits a 34-fold increase in activity without any sacrifice in specificity. Similar stereoselective preferences with S-sulpiride are maintained through ML-guided enzyme engineering as well.

Performing an HSS of the same 64 residues on different substrate pairs provides substantial information to help understand the sequence-fitness landscape of McbA. Remarkably, while many of the substrate pairs contain the same acid or amine, it is difficult to rationalize why certain mutations arise as they are not conserved among many of these enzymes. Across all HSSs, 19 hot spot positions were identified, and each reaction yielded a unique set of hot spots. Between all nine engineered McbA variants, we made a total of 21 different mutations occurring across 14 different residues (FIGS. 22A-22B). This highlights not only the need to interrogate large portions of sequence space for different acid-amine couplings but also the versatility of McbA to be directed to catalyze unique reactions of interest.

One modified amide synthetase McbA enzyme comprises one or more substitutions at amino acid positions P102, T103, V177, I220, C232, A266, A323, or R430 for the production of moclobemide.

Another modified amide synthetase McbA comprising one or more substitutions in combination at amino acid positions P102, T103, V177, C201, A205, C232, A424, or R430 for the production of cinchocaine. Amide synthetase reaction was performed using the method described in Example 4 with the acid and amine indicated in Table 1.

Another modified amide synthetase McbA comprising one or more substitutions in combination at amino acid positions Y97, P102, T103, V177, C201, I220, P221, L225, E228, C232, E244, A266, N316, T319, A323, L412, E423, A424, Y425, or R430 for the production of itopride. Amide synthetase reaction was performed using the method described in Example 4 with the acid and amine indicated in Table 1.

Another modified amide synthetases McbA comprising one or more substitutions in combination at amino acid positions P102, T103, V177, C201, I220, L225, E244, A266, A295, G318, T319, Q320, A323, I421, E423, A424, Y425, R430, or D508 for the production of declopramide. Amide synthetase reaction was performed using the method described in Example 4 with the acid and amine indicated in Table 1.

Another modified amide synthetases McbA comprising one or more substitutions in combination at amino acid positions P102, T103, V177, C201, I220, L225, E244, A266, A295, G318, T319, Q320, A323, I421, E423, A424, Y425, R430, or D508 for the production of metoclopramide. Amide synthetase reaction was performed using the method described in Example 4 with the acid and amine indicated in Table 1.

Another modified amide synthetases McbA comprising one or more substitutions in combination at amino acid positions Y97, P102, T103, V177, C201, I220, P221, L225, E228, C232, E244, A266, N316, T319, A323, L412, E423, A424, Y425, or R430 for the production of trimethobenzamide. Amide synthetase reaction was performed using the method described in Example 4 with the acid and amine indicated in Table 1.

Another modified amide synthetases McbA comprising one or more substitutions in combination at amino acid positions P102, T103, V177, C201, A205, Y209, I220, P221, L225, C232, E244, A266, A295, Y317, G318, T319, Q320, A323, T376, E423, A424, Y425, or R430 for the production of S-sulpride. Amide synthetase reaction was performed using the method described in Example 4 with the acid and amine indicated in Table 1.

Another modified amide synthetases McbA comprising one or more substitutions in combination at amino acid positions P102, T103, V177, C201, I220, L225, E244, A266, A295, G318, T319, Q320, A323, I421, E423, A424, Y425, R430, or D508 for the production of procainamide. Amide synthetase reaction was performed using the method described in Example 4 with the acid and amine indicated in Table 1.

Another modified amide synthetases McbA comprising one or more substitutions in combination at amino acid positions P102, T103, V177, C201, A205, Y209, I220, P221, L225, C232, E244, A266, A295, Y317, G318, T319, Q320, A323, T376, E423, A424, Y425, or R430 for the production of troxipide. Amide synthetase reaction was performed using the method described in Example 4 with the acid and amine indicated in Table 1.

Protein-encoding DNA sequences for McbA variants used in this study are listed below. wt-McbA, the two qm-McbA variants from our ISM engineering campaigns for moclobemide and metoclopramide are included, and the best experimentally validated ML-predicted variant for each compound (designated ml-McbA^{compound}). The Variant ID corresponding to the mutant residues is also included when available. For convenience, the N-terminal CSL-tag (CAT-Strep-Linker fusion containing a strep purification tag) and TEV protease cleavage site on every variant is colored blue and the mutations made relative to wt-McbA are colored red. The amino acid labeling is based off these sequences (i.e., ATG=M1), which notably slightly deviates from previously published work and the crystal structure of McbA (PDB: 6SQ8) that does not include a CSL-tag.

| Enzyme | Nucleotide Sequence |
|---|---|
| wt-McbA | ATGGAGAAAAAAATCTGGAGCCATCCGCAGTTCGAAAAAGGCGGATGGGG |
| SEQ ID NO: 156 | AGAAAACCTGTATTTCCAGGGGGGTTACGCTCGTCGTGTAATGGATGGTAT |
| | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAGTTGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCAGCTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAGCATATAACGTCTATTCCCGTCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| qm-McbA_{moclobemide} | ATGGAGAAAAAAATCTGGAGCCATCCGCAGTTCGAAAAAGGCGGATCCGG |
| SEQ ID NO: 157 | AGAAAACCTGTATTTCCAGGGCGGTTACGCTCGTCGTGTAATGGATGGTAT |
| | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAAGCGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGAGCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCATTTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAGCATATAACGTCTATTCCCTGCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| qm-McbA_{metoclopramide} | ATGGAGAAAAAAATCTGGAGCCATCCGCAGTTCGAAAAAGGCGGATCCGG |
| SEQ ID NO: 158 | AGAAAACCTGTATTTCCAGGGCGGTTACGCTCGTCGTGTAATGGATGGTAT |
| | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAAGCGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCTGCCAGGAAGCATTTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAACCTATAACGTCTATTCCCGTCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| | ATGGAGAAAAAAATCTGGAGCCATCCGCAGTTCGAAAAAGGCGGATCCGG |
| ml-McbA_{moclobemide} | AGAAAACCTGTATTTCCAGGGCGGTTACGCTCGTCGTGTAATGGATGGTAT |
| (SSFT) | CGGTGAAGTAGCGGTAACTGGCGCTGGTGGTTCTGTAACTGGTGCGCGTC |
| SEQ ID NO: 159 | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAAGCGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGAGCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCATTTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAGCATATAACGTCTATTCCACCCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{metoclopramide} | ATGGAGAAAAAAATCTGGAGCCATCCGCAGTTCGAAAAAGGCGGATCCGG |
| (SCFS) | AGAAAACCTGTATTTCCAGGGCGGTTACGCTCGTCGTGTAATGGATGGTAT |
| SEQ ID NO: 160 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAAGCGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCTGCCAGGAAGCATTTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAAGCTATAACGTCTATTCCCGTCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{cinchocaine} | ATGGAGAAAAAAATCTGGAGCCATCCGCAGTTCGAAAAAGGCGGATCCGG |
| (SLFQ) | AGAAAACCTGTATTTCCAGGGCGGTTACGCTCGTCGTGTAATGGATGGTAT |
| SEQ ID NO: 161 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAAGCGTAGCCTTTACTAGCGGCAC |
| | CACTGGCACCCCTAAAGGCGTTGCCCACTCCTCTACCGCTATGAGCGCTT |
| | GTGTGGATGCTCTGGTTTCCATGTACGGTCGCGGTCCTTGGCGTTTCCTG |
| | ATCCCGATCCCTCTGTCTGACCTGGGTGGCGAACTGGCACAGTTTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCAGCTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAGCATATAACGTCTATTCCCAGCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{itopride} | ATGGAGAAAAAAATCTGGAGCCATCCGCAGTTCGAAAAAGGCGGATCCGG |
| (EQLM) | AGAAAACCTGTATTTCCAGGGCGGTTACGCTCGTCGTGTAATGGATGGTAT |
| SEQ ID NO: 162 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAGTTGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGCAGTA |
| | CGGCACCCAGGAAGCACTGTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAATGTATAACGTCTATTCCCGTCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{declopramide} | ATGGAGAAAAAAATCTGGAGCCATCCGCAGTTCGAAAAAGGCGGATCCGG |
| (YSCS) | AGAAAACCTGTATTTCCAGGGCGGTTACGCTCGTCGTGTAATGGATGGTAT |
| SEQ ID NO: 163 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTTATGCTGCTGTTACTGAACAGGCACGCGCAATTGCGGCTGCTGATT |
| | CTGCCGCACTGGTTTTCGAACCAAGCGTTGAAGCTCGTGCAGCTGACCTG |
| | CTGGAACGTGTTTCTGTGCCGGTTGTGCTGTCTCTGGGTCCGACCTCTCG |
| | TGGCCGTGATATCCTGGCAGCTAGCGTTCCGGAAGGTACGCCGCTGCGTT |
| | ACCGTGAACACCCAGAAGGTATCGCAAGCGTAGCCTTTACTAGCGGCACC |
| | ACTGGCACCCCTAAAGGCGTTGCCCACTCCTCTACCGCTATGAGCGCTTG |
| | TGTGGATGCTGCGGTTTCCATGTACGGTCGCGGTCCTTGGCGTTTCCTGA |
| | TCCCGATCCCTCTGTCTGACCTGGGTGGCGAACTGGCACAGTGTACCCTG |
| | GCTACCGGCGGTACCGTTGTGCTGCTGGAAGAGTTCCAACCGGACGCCGT |
| | TCTGGAAGCTATCGAACGTGAACGTGCCACTCACGTGTTCCTGGCGCCGA |
| | ACTGGCTGTACCAGCTGGCTGAACATCCGGCTCTGCCGCGTTCTGATCTG |
| | TCTTCTCTGCGTCGCGTTGTTTACGGCGGTTGCCCGGCAGTACCATCTCGT |
| | GTAGCAGCAGCACGTGAACGTATGGGTGCTGTGCTGATGCAGAACTACGG |
| | CACCCAGGAAGCAGCTTTCATCGCAGCACTGACTCCAGACGATCACGCAC |
| | GTCGTGAACTGCTGACCGCTGTAGGTCGTCCTCTGCCACACGTTGAGGTG |
| | GAAATCCGTGATGACTCTGGTGGTACTCTGCCGCGTGGTGCGGTAGGTGA |
| | AGTCTGGGTACGTTCCCCGATGACTATGTCTGGTTACTGGCGTGACCCGG |
| | AACGTACGGCTCAGGTTCTGTCTGGTGGTTGGCTGCGTACTGGTGATGTT |
| | GGTACCTTCGATGAGGATGGTCACCTGCATCTGACCGATCGTCTGCAGGA |
| | CATCATCATCGTTGAAAGCTATAACGTCTATTCCCGTCGTGTGGAACATGTT |
| | CTGACCGAACACCCAGATGTTCGCGCAGCTGCGGTTGTTGGCGTACCAGA |
| | TCCGGACTCTGGTGAAGCTGTTTGCGCTGCGGTTGTAGTCGCGGATGGTG |
| | CGGATCCTGACCCTGAACACCTGCGTGCTCTGGTTCGTGATCACCTGGGT |
| | GATCTGCACGTTCCTCGCCGTGTTGAGTTCGTTCGCTCCATCCCGGTAACT |
| | CCTGCCGGCAAACCAGATAAAGTGAAAGTGCGTACCTGGTTCACCGACTA |
| | A |
| ml-McbA_{trimethobenzamide} | ATGGAGAAAAAAATCTGGAGCCATCCGCAGTTCGAAAAAGGCGGATCCGG |
| (VFLV) | AGAAAACCTGTATTTCCAGGGCGGTTACGCTCGTCGTGTAATGGATGGTAT |
| SEQ ID NO: 164 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAGTTGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACGTGGGTGGCTTTCTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCACTGTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAGCATATAACGTCTATTCCGTGCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{sulpiride} | ATGGAGAAAAAAATCTGGAGCCATCCGCAGTTCGAAAAAGGCGGATCCGG |
| (ITFV) | AGAAAACCTGTATTTCCAGGGCGGTTACGCTCGTCGTGTAATGGATGGTAT |
| SEQ ID NO: 165 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAGTTGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGACCCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCATTTTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAGCATATAACGTCTATTCCGTGCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{procainamide} | ATGGAGAAAAAAATCTGGAGCCATCCGCAGTTCGAAAAAGGCGGATCCGG |
| (CAMS) | AGAAAACCTGTATTTCCAGGGCGGTTACGCTCGTCGTGTAATGGATGGTAT |
| SEQ ID NO: 166 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAGTTGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCAATGTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAAGCTATAACGTCTATTCCCGTCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |
| ml-McbA_{troxipide} | ATGGAGAAAAAAATCTGGAGCCATCCGCAGTTCGAAAAAGGCGGATCCGG |
| (ALTV) | AGAAAACCTGTATTTCCAGGGCGGTTACGCTCGTCGTGTAATGGATGGTAT |
| SEQ ID NO: 167 | CGGTGAAGTAGCGGTAACTGGCGCTGGTGGTTCTGTAACTGGTGCGCGTC |
| | TGCGCCATCAGGTTCGTCTGCTGGCTCATGCTCTGACCGAAGCGGGTATT |
| | CCGCCAGGCCGTGGTGTAGCATGTCTGCATGCTAACACCTGGCGTGCGAT |
| | CGCACTGCGTCTGGCTGTTCAGGCGATTGGTTGCCACTATGTTGGTCTGC |
| | GTCCTACCGCTGCTGTTACTGAACAGGCACGCGCAATTGCGGCTGCTGAT |
| | TCTGCCGCACTGGTTTTCGAACCAAGCGTTGAAGCTCGTGCAGCTGACCT |
| | GCTGGAACGTGTTTCTGTGCCGGTTGTGCTGTCTCTGGGTCCGACCTCTC |
| | GTGGCCGTGATATCCTGGCAGCTAGCGTTCCGGAAGGTACGCCGCTGCGT |
| | TACCGTGAACACCCAGAAGGTATCGCAGCGGTAGCCTTTACTAGCGGCAC |
| | CACTGGCACCCCTAAAGGCGTTGCCCACTCCTCTACCGCTATGAGCGCTT |
| | GTGTGGATGCTGCGGTTTCCATGTACGGTCGCGGTCCTTGGCGTTTCCTG |
| | ATCCCGATCCCTCTGTCTGACCTGGGTGGCGAACTGGCACAGTGTACCCT |
| | GGCTACCGGCGGTACCGTTGTGCTGCTGGAAGAGTTCCAACCGGACGCC |
| | GTTCTGGAAGCTATCGAACGTGAACGTGCCACTCACGTGTTCCTGGCGCC |
| | GAACTGGCTGTACCAGCTGGCTGAACATCCGGCTCTGCCGCGTTCTGATC |
| | TGTCTTCTCTGCGTCGCGTTGTTTACGGCGGTGCACCGGCAGTACCATCT |
| | CGTGTAGCAGCAGCACGTGAACGTATGGGTGCTGTGCTGATGCAGAACTA |
| | CGGCACCCAGGAAGCACTGTTCATCGCAGCACTGACTCCAGACGATCACG |
| | CACGTCGTGAACTGCTGACCGCTGTAGGTCGTCCTCTGCCACACGTTGAG |
| | GTGGAAATCCGTGATGACTCTGGTGGTACTCTGCCGCGTGGTGCGGTAGG |
| | TGAAGTCTGGGTACGTTCCCCGATGACTATGTCTGGTTACTGGCGTGACC |
| | CGGAACGTACGGCTCAGGTTCTGTCTGGTGGTTGGCTGCGTACTGGTGAT |
| | GTTGGTACCTTCGATGAGGATGGTCACCTGCATCTGACCGATCGTCTGCA |
| | GGACATCATCATCGTTGAAACCTATAACGTCTATTCCGTGCGTGTGGAACA |
| | TGTTCTGACCGAACACCCAGATGTTCGCGCAGCTGCGGTTGTTGGCGTAC |
| | CAGATCCGGACTCTGGTGAAGCTGTTTGCGCTGCGGTTGTAGTCGCGGAT |
| | GGTGCGGATCCTGACCCTGAACACCTGCGTGCTCTGGTTCGTGATCACCT |
| | GGGTGATCTGCACGTTCCTCGCCGTGTTGAGTTCGTTCGCTCCATCCCGG |
| | TAACTCCTGCCGGCAAACCAGATAAAGTGAAAGTGCGTACCTGGTTCACC |
| | GACTAA |

The corresponding amino acid sequences for McbA variants used in this study are listed below.

| Enzyme | Amino Acid Sequence |
|---|---|
| wt-McbA | MEKKIWSHPQFEKGGSGENLYFQGGYARRVMDGIGEVAVTGAGGSVTGARL |
| SEQ ID NO: 2 | RHQVRLLAHALTEAGIPPGRGVACLHANTWRAIALRLAVQAIGCHYVGLRPTA |
| | AVTEQARAIAAADSAALVFEPSVEARAADLLERVSVPVVLSLGPTSRGRDILAA |
| | SVPEGTPLRYREHPEGIAVVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGTQEAAFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVEAYNVYSRRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| qm-McbA_{moclobemide} | MEKKIWSHPQFEKGGSGENLYFQGGYARRVMDGIGEVAVTGAGGSVTGARL |
| SEQ ID NO: 145 | RHQVRLLAHALTEAGIPPGRGVACLHANTWRAIALRLAVQAIGCHYVGLRPTA |
| | AVTEQARAIAAADSAALVFEPSVEARAADLLERVSVPVVLSLGPTSRGRDILAA |
| | SVPEGTPLRYREHPEGIASVAFTSGTTGTPKGVAHSSTAMSACVDAAVSMYG |
| | RGPWRFLIPSPLSDLGGELAQCTLATGGTWVLLEEFQPDAVLEAIERERATHVF |
| | LAPNWLYQLAEHPALPRSDLSSLRRVVYGGAPAVPSRVAAARERMGAVLMQ |
| | NYGTQEAFFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVEAYNVYSLRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| qm-McbA_{metoclopramide} | MEKKIWSHPQFEKGGSGENLYFQGGYARRVMDGIGEVAVTGAGGSVTGARL |
| SEQ ID NO: 146 | RHQVRLLAHALTEAGIPPGRGVACLHANTWRAIALRLAVQAIGCHYVGLRPTA |
| | AVTEQARAIAAADSAALVFEPSVEARAADLLERVSVPVVLSLGPTSRGRDILAA |
| | SVPEGTPLRYREHPEGIASVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGCQEAFFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVETYNVYSRRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{moclobemide} | MEKKIWSHPQFEKGGSGENLYFQGGYARRVMDGIGEVAVTGAGGSVTGARL |
| (SSFT) | RHQVRLLAHALTEAGIPPGRGVACLHANTWRAIALRLAVQAIGCHYVGLRPTA |
| SEQ ID NO: 147 | AVTEQARAIAAADSAALVFEPSVEARAADLLERVSVPVVLSLGPTSRGRDILAA |
| | SVPEGTPLRYREHPEGIASVAFTSGTTGTPKGVAHSSTAMSACVDAAVSMYG |
| | RGPWRFLIPSPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGTQEAFFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVEAYNVYSTRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{metoclopramide} | MEKKIWSHPQFEKGGSGENLYFQGGYARRVMDGIGEVAVTGAGGSVTGARL |
| (SCFS) | RHQVRLLAHALTEAGIPPGRGVACLHANTWRAIALRLAVQAIGCHYVGLRPTA |
| SEQ ID NO: 148 | AVTEQARAIAAADSAALVFEPSVEARAADLLERVSVPVVLSLGPTSRGRDILAA |
| | SVPEGTPLRYREHPEGIASVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGCQEAFFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVESYNVYSRRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{cinchocaine} | MEKKIWSHPQFEKGGSGENLYFQGGYARRVMDGIGEVAVTGAGGSVTGARL |
| (SLFQ) | RHQVRLLAHALTEAGIPPGRGVACLHANTWRAIALRLAVQAIGCHYVGLRPTA |
| SEQ ID NO: 149 | AVTEQARAIAAADSAALVFEPSVEARAADLLERVSVPVVLSLGPTSRGRDILAA |
| | SVPEGTPLRYREHPEGIASVAFTSGTTGTPKGVAHSSTAMSACVDALVSMYG |
| | RGPWRFLIPIPLSDLGGELAQFTLATGGTVVLLEEFQPDAVLEAIERERATHVFL |
| | APNWLYQLAEHPALPRSDLSSLRRVVYGGAPAVPSRVAAARERMGAVLMQN |
| | YGTQEAAFIAALTPDDHARRELLTAVGRPLPHVEVEIRDDSGGTLPRGAVGEV |
| | WVRSPMTMSGYWRDPERTAQVLSGGWLRTGDVGTFDEDGHLHLTDRLQDIII |
| | VEAYNVYSQRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{itopride} | MEKKIWSHPQFEKGGSGENLYFQGGYARRVMDGIGEVAVTGAGGSVTGARL |
| (EQLM) | RHQVRLLAHALTEAGIPPGRGVACLHANTWRAIALRLAVQAIGCHYVGLRPTA |
| SEQ ID NO: 150 | AVTEQARAIAAADSAALVFEPSVEARAADLLERVSVPVVLSLGPTSRGRDILAA |
| | SVPEGTPLRYREHPEGIAVVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | QYGTQEALFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVEMYNVYSRRVEHVLTEHPDVRAAAVVGVPDPDSGEAVCAAVVVADGADP |
| | DPEHLRALVRDHLGDLHVPRRVEFVRSIPVTPAGKPDKVKVRTWFTD |
| ml-McbA_{declopramide} | MEKKIWSHPQFEKGGSGENLYFQGGYARRVMDGIGEVAVTGAGGSVTGARL |
| (YSCS) | RHQVRLLAHALTEAGIPPGRGVACLHANTWRAIALRLAVQAIGCHYVGLRPYA |
| SEQ ID NO: 151 | AVTEQARAIAAADSAALVFEPSVEARAADLLERVSVPVVLSLGPTSRGRDILAA |
| | SVPEGTPLRYREHPEGIASVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGCPAVPSRVAAARERMGAVLMQ |
| | NYGTQEAAFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVESYNVYSRRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{trimethobenzamide} | MEKKIWSHPQFEKGGSGENLYFQGGYARRVMDGIGEVAVTGAGGSVTGARL |
| (VFLV) | RHQVRLLAHALTEAGIPPGRGVACLHANTWRAIALRLAVQAIGCHYVGLRPTA |
| SEQ ID NO: 152 | AVTEQARAIAAADSAALVFEPSVEARAADLLERVSVPVVLSLGPTSRGRDILAA |
| | SVPEGTPLRYREHPEGIAVVAFTSGTTGTPKGVAHSSTAMSACVDAAVSMYG |
| | RGPWRFLIPIPLSDVGGFLAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGTQEALFIAALTPDDHARRELLTAVGRPLPHVEVEIRDDSGGTLPRGAVGE |
| | WWWVRSPMTMSGYWRDPERTAQVLSGGWLRTGDVGTFDEDGHLHLTDRLQDI |
| | IIVEAYNVYSVRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{sulpiride} | MEKKIWSHPQFEKGGSGENLYFQGGYARRVMDGIGEVAVTGAGGSVTGARL |
| (ITFV) | RHQVRLLAHALTEAGIPPGRGVACLHANTWRAIALRLAVQAIGCHYVGLRPTA |
| SEQ ID NO: 153 | AVTEQARAIAAADSAALVFEPSVEARAADLLERVSVPVVLSLGPTSRGRDILAA |
| | SVPEGTPLRYREHPEGIAVVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LTPNWLYQLAEHPALPRSDLSSLRRVVYGGAPAVPSRVAAARERMGAVLMQ |
| | NYGTQEAFFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVEAYNVYSVRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{procainamide} | MEKKIWSHPQFEKGGSGENLYFQGGYARRVMDGIGEVAVTGAGGSVTGARL |
| (CAMS) | RHQVRLLAHALTEAGIPPGRGVACLHANTWRAIALRLAVQAIGCHYVGLRPTA |
| SEQ ID NO: 154 | AVTEQARAIAAADSAALVFEPSVEARAADLLERVSVPVVLSLGPTSRGRDILAA |
| | SVPEGTPLRYREHPEGIAVVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGTQEAMFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVESYNVYSRRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |
| ml-McbA_{troxipide} | MEKKIWSHPQFEKGGSGENLYFQGGYARRVMDGIGEVAVTGAGGSVTGARL |
| (ALTV) | RHQVRLLAHALTEAGIPPGRGVACLHANTWRAIALRLAVQAIGCHYVGLRPTA |
| SEQ ID NO: 155 | AVTEQARAIAAADSAALVFEPSVEARAADLLERVSVPVVLSLGPTSRGRDILAA |
| | SVPEGTPLRYREHPEGIAAVAFTSGTTGTPKGVAHSSTAMSACVDAAVSMYG |
| | RGPWRFLIPIPLSDLGGELAQCTLATGGTVVLLEEFQPDAVLEAIERERATHVF |
| | LAPNWLYQLAEHPALPRSDLSSLRRVVYGGAPAVPSRVAAARERMGAVLMQ |
| | NYGTQEALFIAALTPDDHARRELLTAVGRPLPHVEVEIRDDSGGTLPRGAVGE |
| | VWVRSPMTMSGYWRDPERTAQVLSGGWLRTGDVGTFDEDGHLHLTDRLQDI |
| | IIVETYNVYSVRVEHVLTEHPDVRAAAVVGVPDPDSGEAVCAAVVVADGADPD |
| | PEHLRALVRDHLGDLHVPRRVEFVRSIPVTPAGKPDKVKVRTWFTD |

### Example 5—Modified acyl-CoA Synthetase

The strategy for engineering a formate specific acyl-CoA synthetase was to engineer an acetyl-CoA synthetase to (1) accept the unnatural substrate formate and (2) decrease the activity of the enzyme towards its preferred substrate acetate. Towards this goal, an acetyl-CoA synthetase from Erythobacter sp. was engineered. The engineering workflow consisted of four parts (FIG. 24). First, a site saturated library was generated using the cell-free DNA assembly and protein synthesis library. Second, this library was screened for the desired activity of synthesizing formate from formyl-CoA using an indirect, fluorescent readout. Third, the top variants identified in the screen were further characterized by measuring the amount of soluble protein synthesized. Using this value, a more accurate assay was used to quantify enzyme activity and identify the best variant in the library. This process was iterated several times to accumulate beneficial mutations.

### Assays

The activity of the acyl-CoA synthetase (ACS) variants was first measured towards formate using an enzymatic cascade that produces a fluorescent output. 20-μL CFPS reactions were prepared in 96-well PCR plates (BioRad) with 2.5-μL of LET serving as the DNA template and incubated at 30° C. for 16 hours. After incubation, CFPS reactions were transferred to a 96-well microdialysis plate 3K MW cutoff (Pierce, Thermo Scientific) and dialyzed in 1 L S30 buffer (10 mM Tris pH 8.2, 14 mM magnesium glutamate, and 60 mM potassium glutamate. After 3 hours, fresh buffer was exchanged and the reactions were dialyzed for an additional 12 hours. A reaction master-mix containing 5 mM MgCl₂, 100 mM Tris-HCl pH 7.5, 0.5 mg/mL kanamycin, 2.5 mM Coenzyme A, 5 mM ATP, 0.15 mM thiamine pyrophosphate, 4 mg/mL enriched MeOXC4 extract, and 2 mg/mL enriched HsapGOX extract, and 1 U/mL horse radish peroxidase (Sigma Aldrich) (final reaction concentrations are listed). Enriched extracts were prepared as described below. 74.2-μL of master-mix was added to the dialyzed CFPS reaction containing expressed ACS variants. This reaction was sealed and incubated at 30° C. for 1 hour to reduce background signal in the subsequent assay. Following incubation, 18-μL from each reaction was distributed 4-times into a 384-well black, round-bottom plate (Nunc) using an Integra Viaflo liquid handler—these serve as quadruplicate measurements. The reaction was initiated by adding 7-μL of a reaction substrate-mix containing 100 mM sodium formate, 20 mM formaldehyde, 0.5 mM Ampliflu Red (Sigma Aldrich), and 5 mM ATP (final reaction concentrations are listed), again using an Integra Viaflo. The 384-well plate was immediately transferred to a plate reader (BioTek Syngery 2) and fluorescence was measured at 1 minute intervals for 2 hours with an excitation of 535 nm and emission of 580 nm at 30° C. Enzyme activity was quantified by measuring the rate of fluorescence increase over the first 15 minutes of the reaction.

Enriched extracts were prepared by first transforming a pETBCS expression plasmid encoding either (1) oxalyl-CoA decarboxylase from Methylorubrum extorquens (MeOXC4) or (2) hydroxyacid oxidase from Homo sapiens (HsapGOX). pETBCS plasmid was transformed into chemically competent E. coli BL21 Star (DE3) cells (Invitrogen) following the manufacturer's instructions. Cells were plated onto LB plates containing 100 μg/mL Carbenicillin (LB-Carb) and incubated overnight at 37° C. A single colony was used to inoculate a 5 mL overnight culture of LB-Carb, grown at 37° C. with 250 RPM shaking. The overnight cultures were used to inoculate 1 L of 2×YTPG media (16 g l⁻¹ tryptone, 10 g l⁻¹ yeast extract, 5 g l⁻¹ NaCl, 7 g l⁻¹ potassium phosphate monobasic, 3 g l⁻¹ potassium phosphate dibasic, 18 g l⁻¹ glucose) and grown at 37° C. with 250 RPM shaking. After cells reached OD 0.6, IPTG was added to a final concentration of 0.5 mM and the cells were grown for 4 more hours. Cells were harvested by centrifugation (Beckman Coulter Avanti J-26) at 8,000×g for 10 min at 4° C. Cell pellets were resuspended with cold S30 buffer (10 mM Tris pH 8.2, 14 mM magnesium glutamate, and 60 mM potassium glutamate). Resuspended cells were lysed by homogenization using an Avestin EmulsiFlex-B15 high-pressure homogenizer at 20,000-25,000 psig with a single pass, and the insoluble fraction was removed by centrifugation at 12,000×g for 20 minutes at 4° C. Prepared cell extract was flash frozen in liquid nitrogen and stored at −80° C. until use.

Amino acid sequence for MeOXC4 (SEQ ID NO: 168):
MTVQAQNIDAITAGAMPHEEPELTDGFHLVIDALKLNGIETIYNVPGIP
ITDLGRLAQAEGLRVISFRHEQNAGNAAAIAGFLTKKPGICLTVSAPGF
LNGLTALANATTNCFPMILISGSSEREIVDLQQGDYGEMDQLAIAKPLC
KAAFRVLHAADIGIGVARAIRAAVSGRPGGVYLDLPAKLFSQVIDADLG
ARSLVKVIDAAPAQLPAPAAIARALDVLKSAERPLIILGKGAAYAQADE
AVRALVEESGIPYVPMSMAKGLLPDTHPLSAGAARSTALKDSDVVLLVG
ARLNWLLSHGKGKTWGEPGSKRFIQIDIEPREMDSNVEIVAPVVGDIGS
CVEALLDGIRKDWKGAPSNWLETLRGKREANIAKMAPKLMKNSSPMCFH
SALGALRTVIKERPDAILVNEGCNTLDLARGIIDMYQPRKRLDVGTWGV
MGIGMGFAVAAAVETGKPVLAVEGDSAFGFSGMEVETICRYELPVCIVI
FNNNGIFRGTDTDPTGRDPGTTVFVKNSRYDKMMEAFGGVGVNVTTPDE
LKRAVDEAMNSGKPTLINAEIDPAAGSEGGNIGSLNPQSTLKKK.
Amino acid sequence for HsapGOX (SEQ ID NO: 169):
MLPRLICINDYEQHAKSVLPKSIYDYYRSGANDEETLADNIAAFSRWKL
YPRMLRNVAETDLSTSVLGQRVSMPICVGATAMQRMAHVDGELATVRAC
QSLGTGMMLSSWATSSIEEVAEAGPEALRWLQLYIYKDREVTKKLVRQA
EKMGYKAIFVTVDTPYLGNRLDDVRNRFKLPPQLRMKNFETSTLSFSPE
ENFGDDSGLAAYVAKAIDPSISWEDIKWLRRLTSLPIVAKGILRGDDAR
EAVKHGLNGILVSNHGARQLDGVPATIDVLPEIVEAVEGKVEVFLDGGV
RKGTDVLKALALGAKAVFVGRPIVWGLAFQGEKGVQDVLEILKEEFRLA
MALSGCQNVKVIDKTLVRKNPLAVSKI.

After identifying potential beneficial mutations (“hits”) using the fluorescent assay, the hits were further characterized to confirm activity. First, expression of the top selected variants in CFPS was quantified using radioactive amino acid incorporation. Then, the concentration of the variants was normalized, and an enzymatic assay was assembled. The activity of ACS variants was compared by quantifying the amount of glycolate produced by measurement on GC-MS. CFPS reactions were performed with radioactive 14C-leucine (10 μM) supplemented in addition to all 20 standard amino acids. We used trichloroacetic acid to precipitate radioactive protein samples. Radioactive counts from trichloroacetic acid-precipitated samples were measured by liquid scintillation to quantify soluble and total yields of each protein (MicroBeta2; PerkinElmer). Using the determined concentration of ACS variant, an assay was set up where the concentration of ACS was normalized to 0.6 μM for each variant. CFPS and dialysis was carried out as described above. The 25 μL reaction contained 8 mM magnesium glutamate, 10 mM ammonium glutamate, 10-100 mM sodium formate, 100 mM Bis Tris pH 7, 0.5 mg/mL kanamycin, 5 mM ATP, 0.15 mM thiamine pyrophosphate, 20 mM formaldehyde, 2.5 mM coenzyme A and 0.6 μM ACS variant. The reaction was performed in triplicate in a 96-well PCR plate (BioRad) at 30° C. Reactions were quenched with the addition of 25 μL of a solution of 10% w/v sulfuric acid and 15% w/v NaCl and centrifuged at 4000×G for 10 minutes to precipitate and pellet proteins. 20 μL of supernatant was transferred into a 1.5 mL microcentrifuge tube. Metabolites were extracted by addition of 150 μL of ethyl acetate and vortexing at max speed for 15 minutes. The tubes were then centrifuged at 13,000×G for 3minutes to separate the two phases. 100 μL of ethyl acetate was removed and put into a glass vial insert (Agilent Technologies) and evaporated at 70° C. Samples were derivatized by addition of 20 μL 1:1 pyridine: N,O-Bis(trimethylsilyl)trifluoroacetamide (BSTFA) and incubation at 70° C. for 10 minutes. For GC-MS analysis, samples were analyzed on an Agilent HP-5MS (30 m length×0.25 mm i.d.×0.25 μm film) column with helium carrier gas at constant flow of 1 mL min⁻¹. The inlet temperature was 250° C. and column temperature started at 50° C., held for 2 min, then increased at 60° C. min⁻¹ to 190° C., then increased at 120° C. min⁻¹ to 270° C., and was held for 5 min. Injection volume was 1 μL with a split ratio of 4:1. Concentrations were determined by comparing it to a standard curve of glycolate.Determining apparent steady state kinetics was performed using the same enzymatic cascade as described previously for McbA. Briefly, the activation of formate to formyl-CoA results in the concomitant release of AMP, which can be converted into a measurable signal through NADH oxidation. Additionally, coenzyme A was added at a final concentration of 1 mM.

An embodiment is a modified acyl-CoA synthetase comprising one or more substitutions in combination at amino acid positions V303, T304, L347, 1383, W407, and A523 for the production of formyl-CoA.

Another embodiment is a modified acyl-CoA synthetase comprising one or more substitutions in combination at amino acid positions I300, V303, V379, W407 for the production of formyl-CoA.

Through this work, the enzyme engineering paradigm is shifted from improving the performance of a single transformation towards parallelized protein engineering based on reaction classes. The disclosed protein engineering workflow combines high-throughput, cell-free DNA assembly and protein expression with machine learning to evaluate an easily synthesized, sequence-defined enzyme library, taking large leaps in sequence space in a short period of time, to identify an improved enzyme-moving from wild-type to a quadruple mutant in one week using a single screen. Here, the disclosed protein engineering workflow approach demonstrates non-obvious, improved variants (1.6-fold to 34-fold improvement) can be successfully generated in a parallelized fashion quickly, by navigating nine protein engineering campaigns for the amide synthetase McbA, six of which were performed simultaneously. In doing so, 19 unique residue positions were identified within McbA that influence the production of several molecules, better equipping engineering of McbA towards unique areas of chemical space. The newly generated enzymes would likely differ in substrate scope from wt-McbA, providing new starting points for parallelized enzyme engineering toward new regions of chemical space. Thus, the parallelized protein engineering framework overcomes limitations in traditional directed evolution strategies and provides a manageable method for routine protein engineering.

The speed and flexibility in this new approach offer greater access and exploration to enzyme engineering space. For instance, given a small enough enzyme the HSS could target every residue in an enzyme, allowing a user to collect a comprehensive sequence-fitness landscape for single amino acid mutations. Comprehensive scans of double mutants (as shown in the moclobemide example) are also possible. HSSs can additionally inform higher order library design. For example, if six hot spots were discovered a complex library in which all residue positions were mutated combinatorially may result in more active mutants. Additionally, making the HSS-informed ML strategy iterative could enable the accumulation of dozens of beneficial mutations in a matter of weeks to months. While the predictions of the augmented model are restricted to the residues tested, richer models could be incorporated to extrapolate to larger, untested portions of sequence space. These decisions could be informed by the quality of the data, the number of hotspots, and/or analytic/screening capabilities. One could also imagine performing multiple HSSs that challenge different fitness attributes (e.g., activity, melting temperature, solvent stability, product inhibition, enzyme expression, etc.) and attempting to simultaneously engineer multiple aspects of an enzyme at once.

While this new approach can be general for any enzyme there are limitations and opportunities for improvement. An enzyme target must be compatible with cell-free expression and the molecule must have an analytical method of equivalent throughput. While CFPS has been tuned to express a broad range of challenging proteins⁵⁵⁻⁶⁰, there are cases where this expression system falls short (e.g., multi-subunit eukaryotic proteins, those with complex post-translational modifications, some membrane-bound proteins, oxygen sensitivity). In terms of analytics, the stability of the product must be considered. Given the stability of amide bonds in the presence of the cell-free expression lysate, LC-MS provided a manageable, rapid solution. Even when these constraints are met, there are certainly applications where traditional directed evolution strategies are beneficial (e.g., when a tractable selection method exists, larger jumps in sequence space can be made). Engineering campaigns with different proteins may also warrant exploring various machine learning models and parameters. While excellent performance was shown with the augmented EC model, it is not unreasonable to expect the best augmented model to be protein dependent. Alternative fitness goals may also require alternative fitness predictors. For example, if the goal is to engineer stability, it would reason that a structural-based fitness predictor may be superior. There are numerous additional protein variant effect predictors continuously pushing the state-of-the-art forward that could improve our predictions⁶¹. More complex models based on natural language processing may also outperform linear regression^{12,13}. In any case, the disclosed new method serves as a high-quality data generation tool that can be used by others to contribute to further developing ML strategies.

The workflow described here provides a strong foundation to combine tools in protein engineering and machine learning to begin taking larger, more confident steps in sequence space to generate enzymes with user-defined qualities quickly. The McbA variants discovered through these parallel engineering campaigns demonstrate McbA's potential as an industrially relevant tool for catalyzing the critically important amide bond. Methods to both expedite the enzyme production and screening phase, incorporate simple, effective, and user-friendly machine learning algorithms, and focus on sets of reactions will help move the field forward faster.

It should be noted that while double mutants are exemplified in the figures and examples provided, it is possible to test higher order scans as well (triple, quadruple, etc.). In a higher order scan, every possible combination of mutations at N residues could be tested to achieve the best possible improvements to protein function.

### Systems

A system useful for generating the evolved enzymes is provided. The system may include a computer system, a DNA expression template, and cell-free expression reagents.

An embodiment includes a system comprising a computer system having one or more processors, and memory storing one or more programs for execution by the one or more processors; DNA expression templates comprising nucleic acid sequences encoding variant proteins, wherein the variant proteins are based on a reference protein and wherein the proteins comprise at least one variant amino acid residue as compared to the reference protein; cell-free expression reagents comprising a DNA-dependent RNA polymerase, ribosome, dNTPs, charged tRNAs, and ATP.

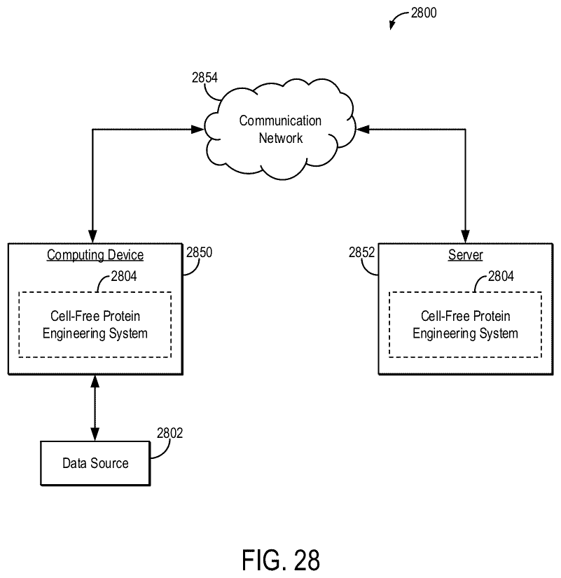

FIG. 28 shows an example of a system 2800 for generating variant or mutant proteins in accordance with some embodiments of the systems and methods described in the present disclosure. As shown in FIG. 28, a computing device 2850 can receive one or more types of data (e.g., DNA expression templates) from data source 2802. In some embodiments, computing device 2850 can execute at least a portion of a cell-free protein engineering system 2804 to generate variant and/or mutant proteins from data received from the data source 2802.

Additionally or alternatively, in some embodiments, the computing device 2850 can communicate information about data received from the data source 2802 to a server 2852 over a communication network 2854, which can execute at least a portion of the cell-free protein engineering system 2804. In such embodiments, the server 2852 can return information to the computing device 2850 (and/or any other suitable computing device) indicative of an output of the cell-free protein engineering system 2804.

In some embodiments, computing device 2850 and/or server 2852 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 2802 can be any suitable source of data (e.g., DNA expression templates, etc.), another computing device (e.g., a server storing DNA expression templates, etc.), and so on. In some embodiments, data source 2802 can be local to computing device 2850. For example, data source 2802 can be incorporated with computing device 2850 (e.g., computing device 2850 can be configured as part of a device for measuring, recording, estimating, acquiring, or otherwise collecting or storing data). As another example, data source 2802 can be connected to computing device 2850 by a cable, a direct wireless link, and so on. Additionally or alternatively, in some embodiments, data source 2802 can be located locally and/or remotely from computing device 2850, and can communicate data to computing device 2850 (and/or server 2852) via a communication network (e.g., communication network 2854).

In some embodiments, communication network 2854 can be any suitable communication network or combination of communication networks. For example, communication network 2854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 2854 can be a local area network, a wide area network, a public network (e.g., the Internet), a private or semi-private network (e.g., a corporate or university intranet), any other suitable type of network, or any suitable combination of networks. Communications links shown in FIG. 28 can each be any suitable communications link or combination of communications links, such as wired links, fiber optic links, Wi-Fi links, Bluetooth links, cellular links, and so on.

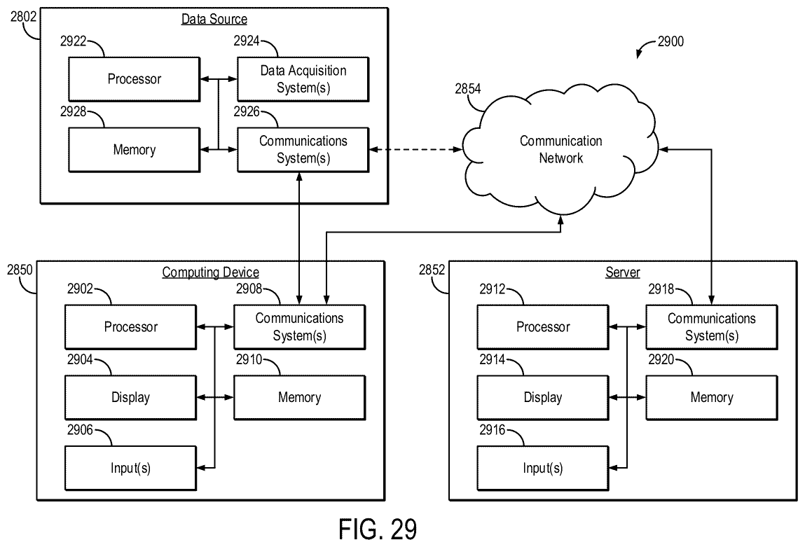

Referring now to FIG. 29, an example of hardware 2900 that can be used to implement data source 2802, computing device 2850, and server 2852 in accordance with some embodiments of the systems and methods described in the present disclosure is shown.

As shown in FIG. 29, in some embodiments, computing device 2850 can include a processor 2902, a display 2904, one or more inputs 2906, one or more communication systems 2908, and/or memory 2910. In some embodiments, processor 2902 can be any suitable hardware processor or combination of processors, such as a central processing unit (“CPU”), a graphics processing unit (“GPU”), and so on. In some embodiments, display 2904 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some embodiments, inputs 2906 can include any suitable input devices and/or sensors that can be used to receive user input, such as a keyboard, a mouse, a touchscreen, a microphone, and so on.

In some embodiments, communications systems 2908 can include any suitable hardware, firmware, and/or software for communicating information over communication network 2854 and/or any other suitable communication networks. For example, communications systems 2908 can include one or more transceivers, one or more communication chips and/or chip sets, and so on. In a more particular example, communications systems 2908 can include hardware, firmware, and/or software that can be used to establish a Wi-Fi connection, a Bluetooth connection, a cellular connection, an Ethernet connection, and so on.

In some embodiments, memory 2910 can include any suitable storage device or devices that can be used to store instructions, values, data, or the like, that can be used, for example, by processor 2902 to present content using display 2904, to communicate with server 2852 via communications system(s) 2908, and so on. Memory 2910 can include any suitable volatile memory, non-volatile memory, storage, or any suitable combination thereof. For example, memory 2910 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some embodiments, memory 2910 can have encoded thereon, or otherwise stored therein, a computer program for controlling operation of computing device 2850. In such embodiments, processor 2902 can execute at least a portion of the computer program to present content (e.g., images, user interfaces, graphics, tables), receive content from server 2852, transmit information to server 2852, and so on. For example, the processor 2902 and the memory 2910 can be configured to perform the methods described herein (e.g., the design, build, test, and learn protein engineering workflows illustrated in FIG. 1A and FIG. 1B).

In some embodiments, server 2852 can include a processor 2912, a display 2914, one or more inputs 2916, one or more communications systems 2918, and/or memory 2920. In some embodiments, processor 2912 can be any suitable hardware processor or combination of processors, such as a CPU, a GPU, and so on. In some embodiments, display 2914 can include any suitable display devices, such as an LCD screen, LED display, OLED display, electrophoretic display, a computer monitor, a touchscreen, a television, and so on. In some embodiments, inputs 2916 can include any suitable input devices and/or sensors that can be used to receive user input, such as a keyboard, a mouse, a touchscreen, a microphone, and so on.

In some embodiments, communications systems 2918 can include any suitable hardware, firmware, and/or software for communicating information over communication network 2854 and/or any other suitable communication networks. For example, communications systems 2918 can include one or more transceivers, one or more communication chips and/or chip sets, and so on. In a more particular example, communications systems 2918 can include hardware, firmware, and/or software that can be used to establish a Wi-Fi connection, a Bluetooth connection, a cellular connection, an Ethernet connection, and so on.

In some embodiments, memory 2920 can include any suitable storage device or devices that can be used to store instructions, values, data, or the like, that can be used, for example, by processor 2912 to present content using display 2914, to communicate with one or more computing devices 2850, and so on. Memory 2920 can include any suitable volatile memory, non-volatile memory, storage, or any suitable combination thereof. For example, memory 2920 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 2920 can have encoded thereon a server program for controlling operation of server 2852. In such embodiments, processor 2912 can execute at least a portion of the server program to transmit information and/or content (e.g., data, images, maps, plots, repots, a user interface) to one or more computing devices 2850, receive information and/or content from one or more computing devices 2850, receive instructions from one or more devices (e.g., a personal computer, a laptop computer, a tablet computer, a smartphone), and so on.

In some embodiments, the server 2852 is configured to perform the methods described in the present disclosure. For example, the processor 2912 and memory 2920 can be configured to perform the methods described herein (e.g., the design, build, test, and learn protein engineering workflows illustrated in FIG. 1A and FIG. 1B).

In some embodiments, data source 2802 can include a processor 2922, one or more data acquisition systems 2924, one or more communications systems 2926, and/or memory 2928. In some embodiments, processor 2922 can be any suitable hardware processor or combination of processors, such as a CPU, a GPU, and so on. Additionally or alternatively, in some embodiments, the one or more data acquisition systems 2924 can include any suitable hardware, firmware, and/or software for coupling to and/or controlling operations of a data acquisition system. In some embodiments, one or more portions of the data acquisition system(s) 2924 can be removable and/or replaceable.

Note that, although not shown, data source 2802 can include any suitable inputs and/or outputs. For example, data source 2802 can include input devices and/or sensors that can be used to receive user input, such as a keyboard, a mouse, a touchscreen, a microphone, a trackpad, a trackball, and so on. As another example, data source 2802 can include any suitable display devices, such as an LCD screen, an LED display, an OLED display, an electrophoretic display, a computer monitor, a touchscreen, a television, etc., one or more speakers, and so on.

In some embodiments, communications systems 2926 can include any suitable hardware, firmware, and/or software for communicating information to computing device 2850 (and, in some embodiments, over communication network 2854 and/or any other suitable communication networks). For example, communications systems 2926 can include one or more transceivers, one or more communication chips and/or chip sets, and so on. In a more particular example, communications systems 2926 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 2928 can include any suitable storage device or devices that can be used to store instructions, values, data, or the like, that can be used, for example, by processor 2922 to control the one or more data acquisition systems 2924, and/or receive data from the one or more data acquisition systems 2924; to generate images, maps, plots, and/or reports from data; present content (e.g., data, images, maps, plots, reports, a user interface) using a display; communicate with one or more computing devices 2850; and so on. Memory 2928 can include any suitable volatile memory, non-volatile memory, storage, or any suitable combination thereof. For example, memory 2928 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 2928 can have encoded thereon, or otherwise stored therein, a program for controlling operation of data source 2802. In such embodiments, processor 2922 can execute at least a portion of the program to generate images, maps, plots, reports, or other data; transmit information and/or content (e.g., data, images, maps, plots, a user interface) to one or more computing devices 2850; receive information and/or content from one or more computing devices 2850; receive instructions from one or more devices (e.g., a personal computer, a laptop computer, a tablet computer, a smartphone, etc.); and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

### REFERENCES

1. Schwander, T., Borzyskowski, L. S. von, Burgener, S., Cortina, N. S. & Erb, T. J. A synthetic pathway for the fixation of carbon dioxide in vitro. Science 354, 900-904 (2016).
2. Lu, H. et al. Machine learning-aided engineering of hydrolases for PET depolymerization. Nature 604, 662-667 (2022).
3. Savile, C. K. et al. Biocatalytic Asymmetric Synthesis of Chiral Amines from Ketones Applied to Sitagliptin Manufacture. Science 329, 305-309 (2010).
4. Arnold, F. H. Directed Evolution: Bringing New Chemistry to Life. Angew. Chem. Int. Ed. 57, 4143-4148 (2018).
5. Arnold, F. H. Design by Directed Evolution. Accounts Chem Res 31, 125-131 (1998).
6. Packer, M. S. & Liu, D. R. Methods for the directed evolution of proteins. Nat Rev Genet 16, 379-394 (2015).
7. Starr, T. N. & Thornton, J. W. Epistasis in protein evolution. Protein Sci 25, 1204-1218 (2016).
8. Miton, C. M. & Tokuriki, N. How mutational epistasis impairs predictability in protein evolution and design. Protein Sci 25, 1260-1272 (2016).
9. Yang, K. K., Wu, Z. & Arnold, F. H. Machine-learning-guided directed evolution for protein engineering. Nat Methods 16, 687-694 (2019).
10. Freschlin, C. R., Fahlberg, S. A. & Romero, P. A. Machine learning to navigate fitness landscapes for protein engineering. Curr Opin Biotech 75, 102713 (2022).
11. Alley, E. C., Khimulya, G., Biswas, S., AlQuraishi, M. & Church, G. M. Unified rational protein engineering with sequence-based deep representation learning. Nat Methods 16, 1315-1322 (2019).
12. Biswas, S., Khimulya, G., Alley, E. C., Esvelt, K. M. & Church, G. M. Low-N protein engineering with data-efficient deep learning. Nat Methods 18, 389-396 (2021).
13. Rives, A. et al. Biological structure and function emerge from scaling unsupervised learning to 250 million protein sequences. P Natl Acad Sci Usa 118, e2016239118 (2021).
14. Madani, A. et al. Large language models generate functional protein sequences across diverse families. Nat Biotechnol 1-8 (2023) doi:10.1038/s41587-022-01618-2.
15. Rao, R. et al. Evaluating Protein Transfer Learning with TAPE. Adv Neur In 32, 9689-9701 (2019).
16. Wittmann, B. J., Yue, Y. & Arnold, F. H. Informed training set design enables efficient machine learning-assisted directed protein evolution. Cell Syst 12, 1026-1045.e7 (2021).
17. Wu, Z., Kan, S. B. J., Lewis, R. D., Wittmann, B. J. & Arnold, F. H. Machine learning-assisted directed protein evolution with combinatorial libraries. Proc National Acad Sci 116, 201901979 (2019).
18. Gelman, S., Fahlberg, S. A., Heinzelman, P., Romero, P. A. & Gitter, A. Neural networks to learn protein sequence-function relationships from deep mutational scanning data. Proc National Acad Sci 118, e2104878118 (2021).
19. Wu, N. C., Dai, L., Olson, C. A., Lloyd-Smith, J. O. & Sun, R. Adaptation in protein fitness landscapes is facilitated by indirect paths. Elife 5, e16965 (2016).
20. Riesselman, A. J., Ingraham, J. B. & Marks, D. S. Deep generative models of genetic variation capture the effects of mutations. Nat Methods 15, 816-822 (2018).
21. Frazer, J. et al. Disease variant prediction with deep generative models of evolutionary data. Nature 599, 91-95 (2021).
22. Hopf, T. A. et al. Mutation effects predicted from sequence co-variation. Nat Biotechnol 35, 128-135 (2017).
23. Russ, W. P. et al. An evolution-based model for designing chorismate mutase enzymes. Science 369, 440-445 (2020).
24. Meier, J. et al. Language models enable zero-shot prediction of the effects of mutations on protein function. in Advances in Neural Information Processing Systems vol. 34 29287-29303 (Curran Associates, Inc., 2021).
25. Luo, Y. et al. ECNet is an evolutionary context-integrated deep learning framework for protein engineering. Nat Commun 12, 5743 (2021).
26. Hsu, C., Nisonoff, H., Fannjiang, C. & Listgarten, J. Learning protein fitness models from evolutionary and assay-labeled data. Nat Biotechnol 1-9 (2022) doi:10.1038/s41587-021-01146-5.
27. Pattabiraman, V. R. & Bode, J. W. Rethinking amide bond synthesis. Nature 480, 471-479 (2011).
28. Bryan, M. C. et al. Key Green Chemistry research areas from a pharmaceutical manufacturers' perspective revisited. Green Chem 20, 5082-5103 (2018).
29. Boström, J., Brown, D. G., Young, R. J. & Keserü, G. M. Expanding the medicinal chemistry synthetic toolbox. Nat Rev Drug Discov 17, 709-727 (2018).
30. Sabatini, M. T., Boulton, Lee. T., Sneddon, H. F. & Sheppard, T. D. A green chemistry perspective on catalytic amide bond formation. Nat Catal 2, 10-17 (2019).
31. Petchey, M. R. & Grogan, G. Enzyme-Catalysed Synthesis of Secondary and Tertiary Amides. Adv. Synth. Catal. 361, 3895-3914 (2019).
32. Lubberink, M., Finnigan, W. & Flitsch, S. L. Biocatalytic amide bond formation. Green Chem (2023) doi:10.1039/d3gc00456b.
33. Wu, S., Snajdrova, R., Moore, J. C., Baldenius, K. & Bornscheuer, U. T. Biocatalysis: Enzymatic Synthesis for Industrial Applications. Angew. Chem. Int. Ed. 60, 88-119 (2021).
34. Chen, Q. et al. Discovery of McbB, an Enzyme Catalyzing the β-Carboline Skeleton Construction in the Marinacarboline Biosynthetic Pathway. Angew. Chem. Int. Ed. 52, 9980-9984 (2013).
35. Petchey, M. R., Rowlinson, B., Lloyd, R. C., Fairlamb, I. J. S. & Grogan, G. Biocatalytic Synthesis of Moclobemide Using the Amide Bond Synthetase McbA Coupled with an ATP Recycling System. Acs Catal 10, 4659-4663 (2020).
36. Petchey, M. et al. The Broad Aryl Acid Specificity of the Amide Bond Synthetase McbA Suggests Potential for the Biocatalytic Synthesis of Amides. Angew. Chem. Int. Ed. 57, 11584-11588 (2018).
37. Hunt, A. C. et al. A high-throughput, automated, cell-free expression and screening platform for antibody discovery. Biorxiv 2021.11.04.467378 (2021) doi:10.1101/2021.11.04.467378.
38. Alford, R. F. et al. The Rosetta All-Atom Energy Function for Macromolecular Modeling and Design. J Chem Theory Comput 13, 3031-3048 (2017).
39. Goldenzweig, A. et al. Automated Structure-and Sequence-Based Design of Proteins for High Bacterial Expression and Stability. Mol Cell 63, 337-346 (2016).
40. Scott, D. J. et al. A Novel Ultra-Stable, Monomeric Green Fluorescent Protein For Direct Volumetric Imaging of Whole Organs Using CLARITY. Sci Rep-uk 8, 667 (2018).
41. Yong, K. J. & Scott, D. J. Rapid directed evolution of stabilized proteins with cellular high-throughput encapsulation solubilization and screening (CHESS). Biotechnol. Bioeng. 112, 438-446 (2015).
42. Pédelacq, J.-D., Cabantous, S., Tran, T., Terwilliger, T. C. & Waldo, G. S. Engineering and characterization of a superfolder green fluorescent protein. Nat Biotechnol 24, 79-88 (2006).
43. Mordhorst, S., Maurer, A., Popadić, D., Brech, J. & Andexer, J. N. A Flexible Polyphosphate-Driven Regeneration System for Coenzyme A Dependent Catalysis. Chemcatchem 9, 4164-4168 (2017).
44. Winn, M. et al. Discovery, characterization and engineering of ligases for amide synthesis. Nature 593, 391-398 (2021).
45. Georgiev, A. G. Interpretable Numerical Descriptors of Amino Acid Space. J Comput Biol 16, 703-723 (2009).
46. Mei, H., Liao, Z. H., Zhou, Y. & Li, S. Z. A new set of amino acid descriptors and its application in peptide QSARs. Peptide Sci 80, 775-786 (2005).
47. Sandberg, M., Eriksson, L., Jonsson, J., Sjöström, M. & Wold, S. New Chemical Descriptors Relevant for the Design of Biologically Active Peptides. A Multivariate Characterization of 87 Amino Acids. J Med Chem 41, 2481-2491 (1998).
48. Barley, M. H., Turner, N. J. & Goodacre, R. Improved Descriptors for the Quantitative Structure-Activity Relationship Modeling of Peptides and Proteins. J Chem Inf Model 58, 234-243 (2018).
49. Laimer, J., Hofer, H., Fritz, M., Wegenkittl, S. & Lackner, P. MAESTRO—multi agent stability prediction upon point mutations. Bmc Bioinformatics 16, 116 (2015).
50. Järvelin, K. & Kekäläinen, J. Cumulated gain-based evaluation of IR techniques. Acm Transactions Information Syst Tois 20, 422-446 (2002).
51. Reetz, M. T., Kahakeaw, D. & Lohmer, R. Addressing the Numbers Problem in Directed Evolution. Chembiochem 9, 1797-1804 (2008).
52. Aslan, A. S., Birmingham, W. R., Karagüler, N. G., Turner, N. J. & Binay, B. Semi-Rational Design of Geobacillus stearothermophilus L-Lactate Dehydrogenase to Access Various Chiral α-Hydroxy Acids. Appl Biochem Biotech 179, 474-484 (2016).
53. Gray, V. E., Hause, R. J. & Fowler, D. M. Analysis of Large-Scale Mutagenesis Data To Assess the Impact of Single Amino Acid Substitutions. Genetics 207, 53-61 (2017).
54. Murphy, L. R., Wallqvist, A. & Levy, R. M. Simplified amino acid alphabets for protein fold recognition and implications for folding. Protein Eng Des Sel 13, 149-152 (2000).
55. Tian, X. et al. Cell-free expression of NO synthase and P450 enzyme for the biosynthesis of an unnatural amino acid L-4-nitrotryptophan. Synthetic Syst Biotechnology 7, 775-783 (2022).
56. Li, J. et al. Cell-free protein synthesis enables high yielding synthesis of an active multicopper oxidase. Biotechnol J 11, 212-218 (2016).
57. Goering, A. W. et al. In Vitro Reconstruction of Nonribosomal Peptide Biosynthesis Directly from DNA Using Cell-Free Protein Synthesis. Acs Synth Biol 6, 39-44 (2017).
58. Koo, C. W., Hershewe, J. M., Jewett, M. C. & Rosenzweig, A. C. Cell-Free Protein Synthesis of Particulate Methane Monooxygenase into Nanodiscs. Acs Synth Biol 11, 4009-4017 (2022).
59. Hershewe, J. M. et al. Improving cell-free glycoprotein synthesis by characterizing and enriching native membrane vesicles. Nat Commun 12, 2363 (2021).
60. Silverman, A. D., Karim, A. S. & Jewett, M. C. Cell-free gene expression: an expanded repertoire of applications. Nat Rev Genet 21, 151-170 (2020).
61. Mansoor, S., Baek, M., Juergens, D., Watson, J. L. & Baker, D. Accurate Mutation Effect Prediction using RoseTTAFold. Biorxiv 2022.11.04.515218 (2022) doi:10.1101/2022.11.04.515218.
62. Kwon, Y.-C. & Jewett, M. C. High-throughput preparation methods of crude extract for robust cell-free protein synthesis. Sci Rep-uk 5, 8663 (2015).
63. Jewett, M. C. & Swartz, J. R. Mimicking the Escherichia coli cytoplasmic environment activates long-lived and efficient cell-free protein synthesis. Biotechnol. Bioeng. 86, 19-26 (2004).
64. Jewett, M. C. & Swartz, J. R. Substrate replenishment extends protein synthesis with an in vitro translation system designed to mimic the cytoplasm. Biotechnol. Bioeng. 87, 465-471 (2004).
65. Kightlinger, W. et al. Design of glycosylation sites by rapid synthesis and analysis of glycosyltransferases. Nat Chem Biol 14, 627-635 (2018).
66. Karim, A. S. et al. In vitro prototyping and rapid optimization of biosynthetic enzymes for cell design. Nat Chem Biol 16, 912-919 (2020).
67. Vornholt, T. et al. Systematic engineering of artificial metalloenzymes for new-to-nature reactions. Sci Adv 7, eabe4208 (2021).
68. Hellberg, S., Sjoestroem, M., Skagerberg, B. & Wold, S. Peptide quantitative structure-activity relationships, a multivariate approach. J Med Chem 30, 1126-1135 (1987).
69. Pettersen, E. F. et al. UCSF ChimeraX: Structure visualization for researchers, educators, and developers. Protein Sci 30, 70-82 (2021).
70. Zubi, Y. S. et al. Metal-responsive regulation of enzyme catalysis using genetically encoded chemical switches. Nat Commun 13, 1864 (2022).

1. A method of generating variant protein with a desired functionality comprising:
(i) generating one or more DNA expression templates comprising nucleic acid sequences encoding a variant protein, wherein the variant protein is based on a reference protein and wherein the protein comprises at least one variant amino acid residue as compared to the reference protein;
(ii) expressing the variant protein using cell-free protein synthesis; and
(iii) analyzing one or more parameters associated with the variant protein and identifying one or more variant proteins with a desired functionality based on the one or more parameters.

2. The method of claim 1, where the one or more DNA expression template is generated by:
(i) having a reference protein on a plasmid that contains nucleotide sequences required for transcription and translation using cell-free protein synthesis;
(ii) introducing a change to the reference protein by PCR amplifying the protein with a primer containing a mutation;
(iii) reassembling the plasmid containing the protein with a variant amino acid;
(iv) amplifying a linear expression template containing the protein with a variant amino acid; and
(v) expressing the variant protein using cell-free protein synthesis.

3. The method of claim 1 or claim 2, further comprising (iv) analyzing the one or more variant proteins to develop one or more computer models based on the at least one variant amino acid residues in the one or more variant proteins.

4. The method of claim 3, further comprising (v) applying the one or more computer models to a sequence of the reference protein to identify at least one predicted variant protein sequence.

5. The method of claim 4, wherein the predicted variant protein sequence does not comprise one of the variant protein generated in step (i).

6. The method of any one of claims 3-5, wherein the one or more computer models comprise machine learning algorithms.

7. A method of generating a variant or mutant protein comprising:
(i) generating one or more DNA expression templates comprising a nucleic acid sequence encoding a variant protein, wherein the variant protein is based on a reference protein and wherein the protein comprises at least one variant amino acid residue as compared to the reference protein;
(ii) expressing the variant protein using cell-free protein synthesis;
(iii) analyzing one or more parameter associated with the variant protein and identifying one or more variant protein with a desired functionality based on the one or more parameter;
(iv) using a computer system having one or more processors, and memory storing one or more programs for execution by the one or more processors: analyzing the one or more variant proteins to develop one or more computer models based on the at least one variant amino acid residues in the one or more variant proteins.

8. The method of claim 7, where the one or more DNA expression template is generated by:
(vi) having a reference protein on a plasmid that contains nucleotide sequences required for transcription and translation using cell-free protein synthesis;
(vii) introducing a change to the reference protein by PCR amplifying the protein with a primer containing a mutation;
(viii) reassembling the plasmid containing the protein with a variant amino acid;
(ix) amplifying a linear expression template containing the protein with a variant amino acid; and
(x) expressing the variant protein using cell-free protein synthesis.

9. The method of claim 7, where analyzing parameters of variant proteins with a desired functionality can be performed directly from cell-free protein synthesis without purification.

10. The method of claim 7, further comprising (v) applying the one or more computer models to a sequence of the reference protein to identify at least one predicted variant protein sequence.

11. The method of claim 8, wherein reassembling the plasmid comprises using Gibson Assembly.

12. A system comprising:
a computer system having one or more processors, and memory storing one or more programs for execution by the one or more processors; DNA expression templates comprising nucleic acid sequences encoding variant proteins, wherein the variant proteins are based on a reference protein and wherein the proteins comprise at least one variant amino acid residue as compared to the reference protein; cell-free expression reagents comprising an information template, an energy regeneration system, and salts.

13. A modified acyl-CoA sythetase (ACS) generated by the methods of any one of claims 1-11, comprising a sequence with one or more amino acid substitutions at amino acid residue A298, I300, W302, V303, T304, I309, V310, S345, L347, Y348, A350, S378, V379, I383, T405, W406, W407, T409, T411, C414, I513, S516, or A523, with reference to
(SEQ ID NO: 1)
MTGFVERPEQAHTPNCTGVQYAAMYERSLADPDGFWLEQAKRLDWTQQP
RKGGEWSYDPVDIKWFADGSLNLCHNAVDRHLDSRGDTPAIIFEPDDPA
TPSRTLTYRQLHSEVIHMANALKAIGVTKGERVTIYMPNIVEGVTAMLA
CARLGAIHSVVFGGFSPEALAGRIIDCESRFVVTADEGKRGAKSVPLKA
NVDAALEVEGVDVTGVLVVQHTGLAVPMTEGRDHWFHEVKSDADVPCET
MAAEDPLFILYTSGSTGKPKGVLHTTGGYGVWTATTFSYIFDYQPGEVF
WCTADIGWVTGHSYIVYGPLQNGATQVLFEGVPNYPDFGRFWDVVAKHK
VSILYTAPTAIRALMREGDDYVTSRDRSSLRLLGSVGEPINPEAWRWYF
DVVGEGRCPIIDTWWQTETGGCMITTLPGAHDMKPGSAGLPMFGIRPQL
VDNDGAVLDGATEGNLCITHSWPGQARSVYGDHDRFVQTYFSTYSGKYF
TGDGCKRDEDGYYWITGRVDDVINVSGHRMGTAEVESALVLHPQVAEAA
VVGYPHDVKGQGIYCYVTTNAGVEGSDELYQELRAHVRKEIGPIATPDQ
IQFTDGLPKTRSGKIMRRILRKVAENDYGSLGDTSTLADPSLVDRLIEG
RQKT

14. The modified ACS of claim 13, wherein the one or more amino acid substitution is selected from an amino acid residue at position V303, T304, L347, I383, W407, or A523.

15. The modified ACS of claim 13, wherein the one or more amino acid substitution is selected from a mutant amino acid residue at position I300, V303, V379, or W407 with reference to SEQ ID NO: 1.

16. The modified ACS of any one of claims 13 to 15, wherein the modified ACS catalyzes an enzymatic process to convert formate into glycolate.

17. The modified ACS of any one of claims 13 to 15, wherein the modified ACS has an altered substrate specificity to convert formate to formyl-CoA.

18. The modified ACS of claim 17, wherein the formate is converted to formyl-CoA in the presence of acetate.

19. A modified amide synthetase (McbA) generated by the methods of any one of claims 1-11, comprising a sequence with one or more amino acid substitutions at amino acid residue Y97, R101, P102, T103, V177, V178, A179, T181, V191, H193, A197, M198, C201, A205, Y209, I220, P221, D224, L225, E228, L229, C232, E244, E245, F246, F264, L265, A266, W269, V292, G293, G294, A295, P296, A297, Q315, N316, Y317, G318, T319, Q320, E321, A323, F324, A341, M375, T376, D400, L412, D414, R415, I421, E423, A424, Y425, N426, R430, L487, P503, A504, G505, K506, P507, or 508 with reference to
(SEQ ID NO: 2)
MEKKIWSHPQFEKGGSGENLYFQGGYARRVMDGIGEVAVTGAGGSVTGA
RLRHQVRLLAHALTEAGIPPGRGVACLHANTWRAIALRLAVQAIGCHYV
GLRPTAAVTEQARAIAAADSAALVFEPSVEARAADLLERVSVPVVLSLG
PTSRGRDILAASVPEGTPLRYREHPEGIAVVAFTSGTTGTPKGVAHSST
AMSACVDAAVSMYGRGPWRFLIPIPLSDLGGELAQCTLATGGTVVLLEE
FQPDAVLEAIERERATHVFLAPNWLYQLAEHPALPRSDLSSLRRVVYGG
APAVPSRVAAARERMGAVLMQNYGTQEAAFIAALTPDDHARRELLTAVG
RPLPHVEVEIRDDSGGTLPRGAVGEVWVRSPMTMSGYWRDPERTAQVLS
GGWLRTGDVGTFDEDGHLHLTDRLQDIIIVEAYNVYSRRVEHVLTEHPD
VRAAAVVGVPDPDSGEAVCAAVVVADGADPDPEHLRALVRDHLGDLHVP
RRVEFVRSIPVTPAGKPDKVKVRTWFTD.

20. The modified McbA of claim 19, wherein the one or more amino acid substitution is selected from a mutant amino acid residue at position P102, T103, V177, 1220, C232, A266, A323, or R430 with reference to SEQ ID NO: 2.

21. The modified McbA of claim 19, wherein the one or more amino acid substitution is selected from a mutant amino acid residue at position P102, T103, V177, C201, A205, C232, A424, or R430 with reference to SEQ ID NO: 2.

22. The modified McbA of claim 19, wherein the one or more amino acid substitution is selected from a mutant amino acid residue at position P102, T103, V177, C201, I220, L225, E244, A266, A295, G318, T319, Q320, A323, I421, E423, A424, Y425, R430, or D508 with reference to SEQ ID NO: 2.

23. The modified McbA of claim 19, wherein the one or more amino acid substitution is selected from a mutant amino acid residue at position Y97, P102, T103, V177, C201, I220, P221, L225, E228, C232, E244, A266, N316, T319, A323, L412, E423, A424, Y425, or R430 with reference to SEQ ID NO: 2.

24. The modified McbA of claim 19, wherein the one or more amino acid substitution is selected from a mutant amino acid residue at position P102, T103, V177, C201, A205, I220, P221, L225, C232, E244, A266, G318, T319, Q320, A323, E423, A424, Y425, or R430 with reference to SEQ ID NO: 2.

25. The modified McbA of claim 19, wherein the one or more amino acid substitution is selected from a mutant amino acid residue at position P102, T103, V177, C201, A205, Y209, I220, P221, L225, C232, E244, A266, A295, Y317, G318, T319, Q320, A323, T376, E423, A424, Y425, or R430.

26. The modified McbA of claim 20 used for the production of moclobemide.

27. The modified McbA of claim 21 used for the production of cinchocaine.

28. The modified McbA of claim 22 used for the production of declopramide, metoclopramide, or procainamide.

29. The modified McbA of claim 23 used for the production of itopride or trimethobenzamide.

30. The modified McbA of claim 25 used for the production of S-sulpride or troxipide.

31. The methods of claim 3 or 10, comprising:
(a) selecting, by a computer system, a plurality of mutations of a protein;
(b) assessing, by the computer system, the plurality of mutations of the protein;
(c) identifying residue locations with the computer system based on the assessed plurality of mutations of the protein;
(d) accessing a machine learning model with the computer system, wherein the machine learning model has been trained on training data to generate data that predict site selection for directed evolution of a protein;
(e) inputting DNA template data associated with the residue locations to the machine learning model, generating predicted site selection data as an output, wherein the predicted site selection data predict residue locations having a selected effect when mutated; and
(f) outputting the predicted site selection data to a user by the computer system.

32. The method of claim 31, wherein step (c) is iteratively performed to identify the residue locations.

33. The method of claim 32, wherein iteratively performing step (c) comprises performing an iterative site saturation mutagenesis to determine additional mutations to assess.
